(19) United States
(12) Patent Application Publication
(10) Pub. No.: US 2022/0040193 A1
(43) Pub. Date: Feb. 10, 2022

(54) FORMULATIONS OF PHOSPHOINOSITIDE 3-KINASE INHIBITORS

(71) Applicant: VENTHERA, INC., Palo Alto, CA (US)

(72) Inventors: Agis KYDONIEUS, Kendall Park, NJ (US); Thomas ROSSI, Portsmouth, NH (US); Hock S. TAN, North Brunswick, NJ (US)

(21) Appl. No.: 17/393,196

(22) Filed: Aug. 3, 2021

Related U.S. Application Data
(60) Provisional application No. 63/060,843, filed on Aug. 4, 2020.
(60) Provisional application No. 63/060,856, filed on Aug. 4, 2020.

(51) Int. Cl. A61K 31/5377; A61K 47/20; A61K 47/10; A61K 47/38; A61K 47/12

(52) U.S. Cl. CPC A61K 31/5377; A61K 47/20; A61K 47/12; A61K 47/38; A61K 47/10

(57) ABSTRACT

The present disclosure provides formulations suitable for topical administration that include compounds of formula (I):

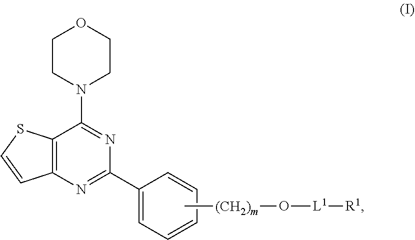

one or more excipients, and one or more optional additional components such as an acid or an antioxidant, wherein subscript m, L¹, and R¹ are as described herein. The formulations provided herein may be sufficiently stable that hydrolysis of the compound of formula (I) in the formulations is less than about 10% under a range of storage conditions. Processes for preparing the formulations and methods of treating vascular malformations with the formulations are also provided herein.

### CROSS-REFERENCES TO RELATED APPLICATIONS

This application claims priority to U.S. Provisional Application No. 63/060,843 filed Aug. 4, 2020 and U.S. Provisional Application No. 63/060,856 filed Aug. 4, 2020, each of which is incorporated herein in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

Vascular anomalies are broadly divided into vascular tumours and vascular malformations. These lesions are composed of abnormal vascular elements of various types, and mainly manifesting and worsening in infants, children, and young adults, and persisting through adulthood. Vascular anomalies may be painful, may be complicated by bleeding, infection, or organ dysfunction, and can have secondary effects on other tissues. Current treatment strategies include surgical excision, pulsed laser, and sclerotherapy, which are invasive, with risks of recurrence. There are growing pharmacological options for these vascular anomalies, but, to date, no specific targeted therapies have been developed.

Vascular malformations comprise abnormal developments of blood vessels that may be found in arteries, veins, arterioles, venules, capillaries, lymphatic channels, and a combination thereof and may occur anywhere in the body. Vascular malformations impacting only veins are referred to as venous malformations, while lymphatic malformations impacting only lymph vessels are referred to as lymphatic malformations. Vascular malformations may be of various types including, for example, port-wine stains (capillary vascular malformations), spider angiomas, venous malformations, lymphatic malformations, arteriovenous malformations, pyogenic granulomas (lobular capillary hemangiomas), hemangiomas, pigmented skin lesions, angiofibromas, and glomangiomas. Vascular malformations can cause cosmetic and/or functional problems including, for example, lumps, birthmarks, pigmented skin lesions, fluid leakage, pain, cardiovascular stress, bleeding, clotting disorders, organ damage, generation of fluid-filled pockets or cysts, and infection. Vascular malformations can be associated with various disorders including Klippel-Trenaunay Syndrome; Parkes-Weber Syndrome; Blue Rubbert Bleb Nevus Syndrome (BRBNS; also referred to as Bean Syndrome); Congenital Lipomatus Overgrowth, Vascular Malformations, Epidermal Nevi and Spine Deformities (CLOVES); Hereditary Hermorrhagic Telangiectasias (HHT; also referred to as Osler-Webe-Rendu Syndrome); Proteus Syndrome; and venolymphatic malformations including angiokeratomas.

Vascular malformations are clinically challenging because current classifications only take into account the patient outcome and the histological characterization. In fact, many efforts are focused on trying to differentiate these lesions from vascular tumors. While vascular benign tumors, such as Infantile Hemangioma, may spontaneously regress and can be treated with propranolol, vascular malformations continue to grow for many years. Venous malformations are of great interest due to current lack of treatment and prognosis. Moreover, pathogenesis of these lesions remain obscure.

The Phosphoinositide 3-Kinase (PI3K) pathway has been extensively studied in tumors due its roles in promoting cellular growth and proliferation. The most common PI3K mutations are in the PIK3CA gene encoding the p110α catalytic subunit, including the “hotspot” activating mutations E545K and H1047R that can lead to constitutive signaling of the pathway. Consequently, activation of the serine/threonine kinase Akt can promote proliferative and cell growth pathways through regulation of mTOR and other intermediates. In addition to driving tumorigenesis, hotspot PIK3CA mutations have also been shown to drive a wide spectrum of non-malignant over-growth disorders collectively termed the PIK3CA-Related Overgrowth Spectrum. More recently, mutations in PIK3CA have been identified in venous malformations (VMs) (Limaye N, et al. Am J Hum Genet. 2015; 97:914-921), the most frequent form of vascular malformations with a frequency of about 1 in 5000 people in the general population. These painful and often disfiguring lesions are characterized by endothelial cell overgrowth, loss of supporting mural cells, and a disorganized extracellular matrix resulting in dilated and distended vessels in a variety of tissues, with common occurrence in the cutaneous layer of the skin (Uebelhoer M, et al. Cold Spring Harb Perspect Med. 2012; 2).

U.S. Pat. No. 6,838,457 discloses that 3-(4-morpholinothieno[3,2-d]pyrimidin-2-yl)phenol has an excellent PI3K inhibiting activity as well as a cancer cell growth inhibiting activity. However, a topical delivery of the compound through the skin for treating vascular malformations by inhibiting the Phosphoinositide 3-Kinase (PI3K) pathway is not known. Considering this, there is urgent need for the development of topical PI3K inhibitors and formulations thereof that can be delivered topically to treat vascular malformations.

### SUMMARY

In a first aspect, the present disclosure provides a formulation including:
a) a compound having formula (I):

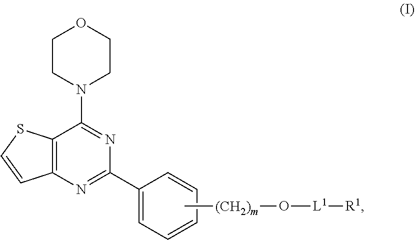

a hydrate, a solvate, a pharmaceutically acceptable salt, or a combination thereof;
wherein:
   subscript m is an integer from 0 to 2;
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) a base formulation including:
   b1) dimethyl sulfoxide (DMSO) in an amount of from about 0% to about 20% by weight of the base formulation;
   b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
   b3) one or more solvents including a C₂₋₆ alkylene glycol, a di-(C₂₋₆ alkylene) glycol, or a combination thereof; and
c) an acid in an amount of no more than about 1% by weight of the base formulation,
   wherein:
a hydrolysis of the compound having formula (I) to a corresponding compound having formula (IV):

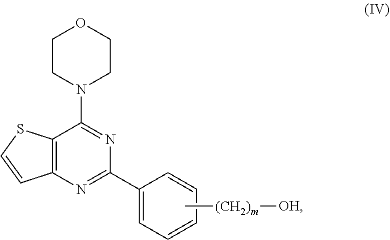

is less than about 10% over a period of about 10 days at a temperature of 80° C. (±2° C.) or over a period of about 6 months at a temperature of 40° C. (±2° C.) and a relative humidity of 75% (±5%), wherein subscript m is an integer from 0 to 2.

In a second aspect, the present disclosure provides a formulation including:
a) a compound having formula (I):

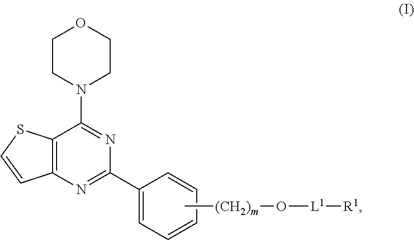

a hydrate, a solvate, a pharmaceutically acceptable salt, or a combination thereof;
wherein:
   subscript m is an integer from 0 to 2;
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) a base formulation including:
   b1) DMSO in an amount of from about 1% to about 20% by weight of the base formulation;
   b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
   b3) one or more solvents including a C₂₋₆ alkylene glycol, a di-(C₂₋₆ alkylene) glycol, or a combination thereof,
      wherein:
the ratio of DMSO to the combined one or more excipients is at least about 1:1 by weight; and
hydrolysis of the compound having formula (I) to a corresponding compound having formula (IV):

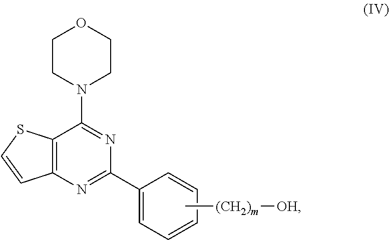

is less than about 10% over a period of about 10 days at a temperature of 80° C. (±2° C.) or over a period of about 6 months at a temperature of 40° C. (±2° C.) and a relative humidity of 75% (±5%), wherein subscript m is an integer from 0 to 2.

In a third aspect, the present disclosure provides a process for preparing a formulation including the compound of formula (I) according to the first aspect and embodiments as described herein. The process includes 1) forming a first mixture including the compound of formula (I) (i.e., a)), components of b) (e.g., components of b1), b2), and b3)), and an acid of c); and 2) mixing the first mixture to form a uniform mixture.

In a fourth aspect, the present disclosure provides a process for preparing a formulation including the compound of formula (I) according to the second aspect and embodiments as described herein. The process includes 1) forming a first mixture including the compound of formula (I) (i.e., a)) and components of b) (e.g., components of b1), b2), and b3)); and 2) sonicating the first mixture to form a second mixture.

In a fifth aspect, the present disclosure provides a formulation, prepared by a process according to the third aspect and embodiments as described herein.

In a sixth aspect, the present disclosure provides a formulation, prepared by a process according to the fourth aspect and embodiments as described herein.

In a seventh aspect, the present disclosure provides a method of treating a vascular malformation through inhibiting phosphoinositide-3-kinase (PI3K) with a formulation including the compound of formula (I), as described herein (e.g., a formulation according to any one of the first, second, fifth, and sixth aspects and embodiments as described herein).

In an eighth aspect, the present disclosure provides a composition including:
a) a compound comprising one or more groups selected from the group consisting of an ester, a lactone, an amide, a lactam, a carbonate, a thiocarbonate, and a carbamate, provided that the amide is other than C(O)NH₂;
b) DMSO in an amount of from about 0% to about 30% by weight;
c) one or more excipients selected from the group consisting of an unsaturated fatty alcohol, an unsaturated fatty acid, an unsaturated fatty ester, an unsaturated fatty ether, and a combination thereof;
d) optionally one or more solvents; and
e) an acid in an amount of no more than about 1% by weight,
   wherein:
the one or more excipients are in an amount of no more than about 10% by weight; and
the compound maintains a relative purity of at least about 90% over a period of about 10 days at a temperature of 80° C. (±2° C.) or over a period of about 6 months at a temperature of 40° C. (±2° C.) and a relative humidity of 75% (±5%).

In a ninth aspect, the present disclosure provides a composition including:
a) a compound including one or more groups selected from the group consisting of an ester, a lactone, an amide, a lactam, a carbonate, a thiocarbonate, and a carbamate, provided that the amide is other than C(O)NH₂;
b) DMSO in an amount of from about 0.1% to about 30% by weight;
c) one or more excipients selected from the group consisting of an unsaturated fatty alcohol, an unsaturated fatty acid, an unsaturated fatty ester, an unsaturated fatty ether, and a combination thereof; and
d) optionally one or more additional excipients,
   wherein
the one or more excipients are in an amount of no more than about 10% by weight;
the ratio of DMSO to the combined one or more excipients is at least about 1:1 by weight; and
the compound maintains a relative purity of at least about 90% over a period of about 10 days at a temperature of 80° C. (±2° C.) or over a period of about 6 months at a temperature of 40° C. (±2° C.) and a relative humidity of 75% (±5%).

### BRIEF DESCRIPTION OF THE DRAWINGS

FIG. 1A to FIG. 1C show the effect of citric acid, acetic acid and phosphoric at various concentrations on stability of compound 1.002 in formulations containing 5% dimethyl sulfoxide (DMSO). FIG. 1A: citric acid; FIG. 1B: acetic acid; and FIG. 1C: phosphoric acid.

FIG. 2 shows the effect of citric acid at various concentrations on stability of compound 1.002 in formulations containing 5% DMSO.

FIG. 3 shows the effect of peroxide values at day 0 on stability of compound 1.002 at Day 10.

FIG. 4 shows skin permeation of compound 1.002 in formulations (BA1-1), (BB1-1), (BB1-4), and (BB1-6).

FIG. 5 shows incidence of erythema in minipigs treated with formulations (13-1) and (13-2).

FIG. 6 shows incidence of other dermal observations in minipigs treated with formulations (13-1) and (13-2).

### DETAILED DESCRIPTION OF THE DISCLOSURE

### I. General

The present disclosure provides a formulation including a compound of formula (I) and an acid (e.g., citric acid) in an amount of no more than about 1% by weight of the base formulation (i.e., a total weight of a formulation without the compound of formula (I), the acid, and a gelling agent), where the hydrolysis of the compound of formula (I) in the formulation is less than about 10% under described storage conditions. After topical delivery, the compounds of the present disclosure are substantially converted to the corresponding compounds of formula (IV) that are capable of inhibiting one or more of the phosphoinositide 3-kinase enzymes, which are part of the PI3K/AKT pathway, thereby providing beneficial therapeutic effects for the treatment of vascular malformations. The present formulations with a low amount of an acid exhibit improved stability of the compound of formula (I). A process for preparing the formulations of the present disclosure is also provided herein.

The present disclosure further provides a formulation including a compound of formula (I), where dimethyl sulfoxide (DMSO) is present in the formulation in an amount of from about 1% to about 20% by weight of the base formulation (i.e., a total weight of a formulation without the compound of formula (I) and a gelling agent) and where the hydrolysis of the compound of formula (I) in the formulation is less than about 10% under described storage conditions. After topical delivery, the compounds of the present disclosure are substantially converted to the corresponding compounds of formula (IV) that are capable of inhibiting one or more of the phosphoinositide 3-kinase enzymes, which are part of the PI3K/AKT pathway, thereby providing beneficial therapeutic effects for the treatment of vascular malformations. A process for preparing the formulations of the present disclosure is also provided herein.

The formulations provided herein may provide reduced skin irritation (e.g., erythema, edema, etc.) as compared to formulations containing higher amounts of DMSO, such as at least about 30% DMSO, when applied topically while still providing substantial skin permeability.

The formulations provided herein may provide reduced skin irritation (e.g., erythema, edema, etc.) as compared to other formulations, while still providing substantial skin permeability.

The present disclosure provides a composition including a compound that is susceptible to hydrolysis due to hydroperoxides present in the composition. The present composition is achieved by addition of an acid in an amount of no more than about 1% by weight and/or by addition of DMSO. The composition can be a formulation including a compound of formula (I) for the treatment of vascular malformations.

The present disclosure also provides methods of treating vascular malformations by inhibiting the PI3K/AKT pathway with the formulations of the present disclosure.

### II. Definitions

The abbreviations used herein have their conventional meaning within the chemical and biological arts.

“Alkyl” refers to a straight or branched, saturated, aliphatic radical having the number of carbon atoms indicated (i.e., C₁₋₆ means one to six carbon atoms). Alkyl can include any number of carbon atoms, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 24 carbon atoms, such as, but not limited to heptyl, octyl, nonyl, decyl, etc.

“Alkenyl” refers to a straight chain or branched hydrocarbon having at least 2 carbon atoms and at least one double bond and having the number of carbon atom indicated (i.e., C₂₋₆ means to two to six carbon atoms). Alkenyl can include any number of carbon atoms, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₅, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Hydroxyalkyl” refers to an alkyl group, as defined above, where at least one of the hydrogen atoms is replaced with a hydroxy group. As for the alkyl group, a hydroxyalkyl or alkylhydroxy groups can have any suitable number of carbon atoms, such as C₁-C₆. Exemplary hydroxyalkyl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxy ethyl, and the like.

“Haloalkyl” refers to alkyl, as defined above, where some or all of the hydrogen atoms are replaced with halogen atoms. As for alkyl group, haloalkyl groups can have any suitable number of carbon atoms, such as C₁-C₆. For example, haloalkyl includes trifluoromethyl, fluoromethyl, 2,2,2-trifluoroethyl, etc. In some instances, the term “perfluoro” can be used to define a compound or radical where all the hydrogens are replaced with fluorine. For example, perfluoromethyl refers to 1,1,1-trifluoromethyl.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl.

“Aryl-alkyl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbon atoms, such as C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The aryl component is as defined above. Examples of aryl-alkyl groups include, but are not limited to, benzyl (phenyl-CH₂—). Aryl-alkyl groups can be substituted or unsubstituted.

“Aryl-alkenyl” refers to a radical having both an alkenyl component and an aryl component, where the alkenyl component links the aryl component to the point of attachment. The alkenyl component is as defined above, except that the alkenyl component is at least divalent, an alkenylene, to link to the aryl component and to the point of attachment. The alkenyl component can include any number of carbon atoms, such as C₂, C₂₋₃, C₂₋₄, C₂₋₅, C₂₋₆, C₂. 8, C₃, C₃₋₄, C₃₋₅, C₃₋₆, C₄, C₄₋₅, C₄₋₅, C₅, C₅₋₆, and C₆. The alkenyl component can have any suitable number of double bonds, including, but not limited to, 1, 2, 3, 4, 5 or more. The aryl component is as defined above. Examples of aryl-alkenyl groups include, but not limited to phenyl-CH═CH—. Aryl-alkenyl groups can be substituted or unsubstituted.

“Alkylene glycol” refers to a compound having the formula of HO-[alkylene-O]—H, wherein the alkylene group has, e.g., 2 to 6, 2 to 4, or 2 to 3 carbon atoms. In some embodiments, the alkylene glycol is a C₂₋₆ alkylene glycol. In some embodiments, the C₂₋₆ alkylene glycol is propylene glycol (1,2-propanediol, also referred to herein as PG).

“Di-alkylene glycol” refers to a compound having the formula of HO-(alkylene-O)₂—H, wherein the alkylene group has, e.g., 2 to 6, 2 to 4, or 2 to 3 carbon atoms. In some embodiments, the di-alkylene glycol is a di-(C₂₋₆ alkylene) glycol. In some embodiments, the di-(C₂₋₆ alkylene) glycol is dipropylene glycol (DPG). Dipropylene glycol can include one or more isomers, for example 4-oxa-2,6-heptandiol, 2-(2-hydroxy-propoxy)-propan-1-ol, 2-(2-hydroxy-1-methyl-ethoxy)-propan-1-ol, and 3,3′-oxybis(propan-1-ol).

“Polyethylene glycol” refers to a polymer having the formula of HO—(CH₂CH₂O)ₙ—OH with variations in subscript “n”. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present disclosure can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to PEG200, PEG300, PEG400, PEG600, and PEG900. The number following the “PEG” in the name refers to the average molecular weight of the polymer.

“Unsaturated fatty alcohol” refers to a primary alcohol with a long aliphatic chain, having one or more C═C double bonds. The C═C double bonds can give either cis or trans isomers. A cis configuration means that the two hydrogen atoms adjacent to the double bond lie on the same side of the chain. A trans configuration, by contrast, means that the adjacent two hydrogen atoms lie on opposite sides of the chain. The unsaturated fatty alcohol can include 8 to 24 carbon atoms. The unsaturated fatty alcohol includes, but is not limited to, palmitoleyl alcohol, oleyl alcohol (unsaturated), and erucyl alcohol.

“Unsaturated fatty acid” refers to a carboxylic acid with a long aliphatic chain having one or more C═C double bonds. The C═C double bonds can provide either cis or trans isomers. A cis configuration means that the two hydrogen atoms adjacent to the double bond lie on the same side of the chain. A trans configuration, by contrast, means that the adjacent two hydrogen atoms lie on opposite sides of the chain. Unsaturated fatty acid can include, e.g., a chain of 10 to 24 carbon atoms. The unsaturated fatty acid includes mono-unsaturated fatty acids, di-unsaturated fatty acids, and poly-unsaturated fatty acids.

Mono-unsaturated fatty acids include, but are not limited to, caproleic acid, lauroleic acid, myristoleic acid, palmitoleic acid, sapienic acid, oleic acid, elaidic acid, vaccenic acid, gadoleic acid, eicosenoic acid, erucic acid, brassidic acid, and nervonic acid. In some embodiments, the unsaturated fatty acid having 10-18 carbon atoms is caproleic acid, lauroleic acid, myristoleic acid, palmitoleic acid, sapienic acid, oleic acid, elaidic acid, vaccenic acid, linoleic acid, alpha-linolenic acid, gamma-linolenic acid, columbinic acid, pinolenic acid, or stearidonic acid.

Di-unsaturated fatty acids include, but are not limited to, linoleic acid, eicosadienoic acid, and docosadienoic acid. The di-unsaturated fatty acid having 18 carbon atoms is linoleic acid.

Poly-unsaturated fatty acids include, but are not limited to, alpha-linolenic acid, gamma-linolenic acid, columbinic acid, pinolenic acid, eleostearic acid, beta-eleostearic acid, mead acid, dihomo-γ-linolenic acid, eicosatrienoic acid, stearidonic acid, arachidonic acid, eicosapentaenoic acid, docosapentaenoic acid, and docosahexaenoic acid. In some embodiments, the poly-unsaturated fatty acid having 18 carbon atoms is alpha-linolenic acid, gamma-linolenic acid, columbinic acid, pinolenic acid, or stearidonic acid.

“Unsaturated fatty ester” refers to a type of ester that results from the combination of an unsaturated fatty alcohol with an acid, an unsaturated fatty acid with an alcohol, or an unsaturated fatty acid with an unsaturated fatty alcohol, wherein the unsaturated fatty alcohol and unsaturated fatty acid are as defined above.

“Unsaturated fatty ether” refers to a type of ether that results from the combination of an unsaturated fatty alcohol with an alcohol. The alcohol can include a polyethylene glycol. The unsaturated fatty ethers include, but are not limited to, a polyoxyethylene oleyl ether derived from oleyl alcohol and a polyethylene glycol. The polyethylene glycol useful for deriving the oleyl ether include, but are not limited to, those described herein. Suitable Polyoxyethylene oleyl ethers include, but are not limited to, polyoxyethylene (10) oleyl ether (Brij 96/Brij O10).

“Hydroperoxide”, “peroxol”, or “peroxide” refers to a compound containing the peroxide functional group (ROOR′) or the hydroperoxide functional group (ROOH), wherein R and R′ can be, for example alkyl, alkenyl, aryl, etc.

“Ester” refers a compound containing a functional group of —C(O)O—; and “lactone” refers to an ester where the —C(O)O— group is a part of a ring.

“Amide” refers a compound containing a functional group of —C(O)NH— or —C(O)N—; and “lactam” refers to an amide where the —C(O)NH— group is a part of a ring.

“Carbonate” refers to a compound containing a functional group of —OC(O)O—; “thiocarbonate” refers to a compound containing a functional group of —OC(O)S—; and “carbamate” refers to a compound containing a functional group of —OC(O)NH—.

“Solvate” refers to a compound provided herein or a salt thereof, that further includes a stoichiometric or non-stoichiometric amount of solvent bound by non-covalent intermolecular forces. The solvent herein refers to non-water solvent.

“Hydrate” refers to a compound that is complexed with a water molecule. The compounds of the present disclosure can be complexed with U water molecule or from 1 to 10 water molecules.

“Composition” and “formulation” as used herein is intended to encompass a product comprising the specified ingredients in the specified amounts, as well as any product, which results, directly or indirectly, from combination of the specified ingredients in the specified amounts. By “pharmaceutically acceptable” it is meant a carrier, diluent or excipient included in the composition (or formulation) must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors and colors. Pharmaceutical excipients useful in the present disclosure for transdermal/topical delivery include, but are not limited to, enhancers, solubilizers, antioxidants, plastisizers, thickeners, polymers, and pressure sensitive adhesives. Additional pharmaceutical excipients may also be useful in the present disclosure.

“MTPP” refers to 3-(4-morpholinothieno(3,2-d)pyrimidin-2-yl)phenol (also as the compound of formula (IVa)).

“Base formulation” refers to a formulation without a compound of formula (I) or 3-(4-morpholinothieno(3,2-d)pyrimidin-2-yl)phenol (abbreviated as MTPP), an acid (when present), and a gelling agent.

“Weight of the base formulation” refers to a total weight of a formulation without a compound of formula (I) or 3-(4-morpholinothieno(3,2-d)pyrimidin-2-yl)phenol (abbreviated as MTPP), an acid (when present), and a gelling agent.

“DMSO is present in an amount of about x % to about y % by weight of the base formulation” refers to DMSO present in an amount of about x % to about y % by weight as compared to the total weight of the base formulation without a compound of formula (I), an acid (when present), and a gelling agent. This term applies to C₁₋₃ alkyl-(OCH₂CH₂)₁₋₅—OH (e.g, 2-(2-ethoxyethoxy)ethanol), a C₂₋₆alkylene glycol (e.g., propylene glycol), a di-(C₂₋₆ alkylene) glycol (e.g., dipropylene glycol), a polyethylene glycol (e.g., PEG400), one or more excipients (e.g., an unsaturated fatty alcohol, an unsaturated fatty acid, an unsaturated fatty ester, and an unsaturated fatty ether), and an acid (e.g., citric acid), when referring to an amount present by weight of the base formulation.

“The compound of formula (I) is present in an amount of about x % by weight of the base formulation” refers the weight percentage of the compound of formula (I) as compared to the total weight of the formulation without the compound of formula (I), an acid (when present), and a gelling agent.

“The gelling agents is present in an amount of about x % to about y % by weight of the base formulation” refers the weight percentage of the gelling agent as compared to the total weight of the formulation without a compound of formula (I), an acid (when present), and the gelling agent. For example, “the hydroxypropyl cellulose is present in an amount of about x % to about y % by weight of the base formulation” refers the weight percentage of the hydoxypropyl cellulose as compared to the total weight of the base formulation without a compound of formula (I), an acid (when present), and the hydoxypropyl cellulose.

“Degree to saturation” refers to a relative content of the compound of formula (I) in the formulation when an amount of the compound of formula (I) is compared to a saturated amount of the compound of formula (I) in the same formulation. The compound of formula (I) in the formulation can have a degree to saturation of from 1% to 100%. 100% degree to saturation means that the compound of formula (I) is present in a saturated amount in the formulation.

“A relative purity of the compound of formula (I) in the formulation” refers to the purity of the compound of formula (I) at a certain time point (e.g., day 10) stored under stressed conditions (e.g., 80° C.) or under normal storage conditions (e.g., room temperature) as compared to an initial purity of the compound of formula (I) at time zero (i.e., day 0). As always, the relative purity of the compound of formula (I) at time zero (i.e., day 0) is set as 100%.

“Inhibition”, “inhibits” and “inhibitor” refer to a compound that prohibits or a method of prohibiting, a specific action or function. Inhibition may be complete (e.g., 100% inhibition) or partial (e.g., less than 100% inhibition, such as at least about 10%, 20%, 30%, 40%, 50%, 60%, 70%, 80%, 90%, 91%, 92%, 93%, 94%, 95%, 96%, 97%, 98%, 99%, or higher).

“Administering” refers to providing a composition or formulation to a subject (e.g., a patient, such as a human patient) via a desired route, such as via topical administration. Topical administration may comprise, for example, application of a composition in the form of a foam, a lotion, a spray, an aerosol, an ointment, a cream, a gel, a paste, a patch, or an in-situ patch to a surface of a subject, such as to the skin of the subject. The area over which the composition is applied may vary based upon, e.g., the condition of the subject (e.g., the area and severity of a vascular malformation such as a venous malformation) as well as the characteristics of the composition (e.g., form, drug load, etc.).

“In-situ patch” refers to the material formed when the formulation is sprayed or otherwise applied to a surface of a subject (e.g., to the skin of the subject), one or more times and thus allowed to develop a thickness. The thickness of an in-situ patch may range from about 1 to about 6 millimeters (mm).

“Topical” means application of a suitable compound (e.g., active agent) or composition comprising a compound (e.g., active agent) to a surface of a subject (e.g., the skin of the subject) to treat diseases or conditions, for example vascular malformation (e.g., venous malformation). In some embodiments, “topical” means application of a suitable compound (e.g., active agent) or composition comprising a compound (e.g., active agent) to the skin with adequate penetration of stratum corneum to reach (or move through) the epidermis or dermis to treat the vascular malformation. In some embodiments of topical application, the compound or composition penetrates stratum corneum and therefore reaches (or moves through) the epidermis or dermis without significant systemic exposure nor intent to treat or prevent a disease of another organ system. In some embodiments of topical application, the compound or composition is delivered transdermally across the skin for systemic distribution. Examples include transdermal patches used for drug delivery.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; and improving a patient's physical or mental well-being. The treatment or amelioration of symptoms can be based on objective or subjective parameters; including the results of a physical examination, assay (e.g., analysis of a fluid or tissue of a subject, such as blood, plasma, or urine), imaging analysis, neuropsychiatric exams, and/or a psychiatric evaluation.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, the patient is human.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, safety, and response of the subject, and will be ascertainable by clinicians, pharmacists, and the like (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“A,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Formulations

### IIIA. Formulations Including an Acid

In a first aspect, the present disclosure provides a formulation including:
a) a compound having formula (I):

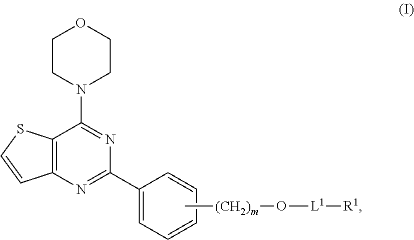

a hydrate, a solvate, a pharmaceutically acceptable salt, or a combination thereof;
wherein:
   subscript m is an integer from 0 to 2;
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) a base formulation including:
   b1) dimethyl sulfoxide (DMSO) in an amount of from about 0% to about 20% by weight of the base formulation;
   b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
   b3) one or more solvents including a C₂₋₆ alkylene glycol, a di-(C₂₋₆ alkylene) glycol, or a combination thereof; and
c) an acid in an amount of no more than about about 1% by weight of the base formulation, wherein:
   a hydrolysis of the compound having formula (I) to a corresponding compound having formula (IV):

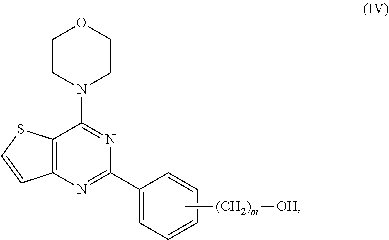

is less than about 10% over a period of about 10 days at a temperature of 80° C. (±2° C.) or over a period of about 6 months at a temperature of 40° C. (±2° C.) and a relative humidity of 75% (±5%), wherein subscript m is an integer from 0 to 2.

Without being bound to a particular theory, it is believed that an unsaturated fatty alcohol and acid (e.g., oleyl alcohol and oleic acid) and an ester or ether containing the unsaturated sidechain of an unsaturated fatty alcohol and acid (e.g., oleyl alcohol and/or oleic acid) can react with oxygen from the air in a radical chain process to convert the internal double bond to a family of allylic hydroperoxides. The extent of hydroperoxide formation in oleyl alcohol may depends on the age and source of the oleyl alcohol, with older and more oxygen-exposed batches expected to exhibit more of the allylic hydroperoxide impurities. The hydroperoxyl functional group is a particularly active nucleophile towards the ester carbonyl, and in the presence of water actively promotes the hydrolysis of aryl esters.

Without further being bound to a particular theory, it is believed that an acid (e.g., citric acid) in a suitable amount can interfere with the deprotonation of hydroperoxides, thereby reducing the formation of hydroperoxyl species as the active nucleophile towards the hydrolysis of an ester.

The formulation can be a topical formulation suitable for a topical application. In some embodiments, the formulation is a topical formulation. In some embodiments, the formulation is suitable for a topical application.

### IIIA-1. Excipients

In some embodiments, one or more excipients include an unsaturated fatty alcohol. In some embodiments, one or more excipients are an unsaturated fatty alcohol.

Unsaturated fatty alcohol as defined herein can include, e.g., a chain of 10 to 24 carbon atoms. In some embodiments, the unsaturated fatty alcohol is in a mixture of different unsaturated fatty alcohols. Suitable fatty alcohols include, but are not limited to, palmitoleyl alcohol, oleyl alcohol, and erucyl alcohol. In some embodiments, the unsaturated fatty alcohol is palmitoleyl alcohol, oleyl alcohol, erucyl alcohol, or a combination thereof. In some embodiments, the unsaturated fatty alcohol includes oleyl alcohol. In some embodiments, the unsaturated fatty alcohol is oleyl alcohol.

In some embodiments, the one or more excipients include oleyl alcohol. In some embodiments, one or more excipients are oleyl alcohol.

In some embodiments, the one or more excipients include an unsaturated fatty acid. In some embodiments, one or more excipients are an unsaturated fatty acid.

Unsaturated fatty acid as defined herein can include, e.g., a chain of 10 to 24 carbon atoms. The unsaturated fatty acid as defined herein can include mono-unsaturated fatty acids, di-unsaturated fatty acids, poly-unsaturated fatty acids, or a combination thereof. In some embodiments, the unsaturated fatty acid is in a mixture of different unsaturated fatty acids. In some embodiments, the unsaturated fatty acid is a mono-unsaturated fatty acid, a di-unsaturated fatty acid, a poly-unsaturated fatty acid, or a combination thereof. In some embodiments, the unsaturated fatty acid has between about 10 to about 18 carbon atoms on average. In some embodiments, the unsaturated fatty acid has about 12-20, 14-20, 12-18, 14-18, or 16-18 carbon atoms on average.

In some embodiments, the unsaturated fatty acid is a mono-unsaturated fatty acid. In some embodiments, the unsaturated fatty acid is a mono-unsaturated fatty acid having 10-18 carbon atoms. Suitable mono-unsaturated fatty acids include, but are not limited to, caproleic acid, lauroleic acid, myristoleic acid, palmitoleic acid, sapienic acid, oleic acid, elaidic acid, vaccenic acid, gadoleic acid, eicosenoic acid, erucic acid, brassidic acid, and nervonic acid. In some embodiments, the unsaturated fatty acid is caproleic acid, lauroleic acid, myristoleic acid, palmitoleic acid, sapienic acid, oleic acid, elaidic acid, vaccenic acid, linoleic acid, alpha-linolenic acid, gamma-linolenic acid, columbinic acid, pinolenic acid, stearidonic acid, or a combination thereof. In some embodiments, the unsaturated fatty acid is caproleic acid, lauroleic acid, myristoleic acid, palmitoleic acid, sapienic acid, oleic acid, or a combination thereof. In some embodiments, the unsaturated fatty acid is myristoleic acid, palmitoleic acid, oleic acid, or a combination thereof. In some embodiments, the unsaturated fatty acid includes oleic acid. In some embodiments, the unsaturated fatty acid is oleic acid.

In some embodiments, the unsaturated fatty acid is a di-unsaturated fatty acid. Di-unsaturated fatty acids include, but are not limited to, linoleic acid, eicosadienoic acid, and docosadienoic acid. In some embodiments, the unsaturated fatty acid is linoleic acid, eicosadienoic acid, docosadienoic acid, or a combination thereof. In some embodiments, the unsaturated fatty acid is linoleic acid.

In some embodiments, the unsaturated fatty acid is a poly-unsaturated fatty acid. Poly-unsaturated fatty acids include, but are not limited to, alpha-linolenic acid, gamma-linolenic acid, columbinic acid, pinolenic acid, eleostearic acid, beta-eleostearic acid, mead acid, dihomo-γ-linolenic acid, eicosatrienoic acid, stearidonic acid, arachidonic acid, eicosapentaenoic acid, docosapentaenoic acid, and docosahexaenoic acid. In some embodiments, the unsaturated fatty acid is alpha-linolenic acid, gamma-linolenic acid, columbinic acid, pinolenic acid, stearidonic acid, or a combination thereof.

In some embodiments, one or more excipients include an unsaturated fatty ester, wherein the unsaturated fatty ester is formed from an unsaturated fatty alcohol with an acid, an unsaturated fatty acid with an alcohol, or an unsaturated fatty acid with an unsaturated fatty alcohol; and the unsaturated fatty alcohol and unsaturated fatty acid are as defined and described herein. In some embodiments, one or more excipients are an unsaturated fatty ester, wherein the unsaturated fatty ester is formed from an unsaturated fatty alcohol with an acid, an unsaturated fatty acid with an alcohol, or an unsaturated fatty acid with an unsaturated fatty alcohol; and the unsaturated fatty alcohol and unsaturated fatty acid are as defined and described herein. In some embodiments, the unsaturated fatty ester is an ester of oleyl alcohol, an ester of oleic acid, or a combination thereof. In some embodiments, the unsaturated fatty ester includes an ester of oleyl alcohol. In some embodiments, the unsaturated fatty ester is an ester of oleyl alcohol.

In some embodiments, one or more excipients include an unsaturated fatty ether, wherein the unsaturated fatty ether is formed from an unsaturated fatty alcohol with an alcohol. In some embodiments, one or more excipients are an unsaturated fatty ether, wherein the unsaturated fatty ether is formed from an unsaturated fatty alcohol with an alcohol. In some embodiments, the unsaturated fatty ether is an ether of oleyl alcohol. The alcohol can include a polyethylene glycol. In some embodiments, the alcohol is a polyethylene glycol. Exemplified unsaturated fatty ethers include a polyoxyethylene oleyl ether, for example polyoxyethylene (10) oleyl ether (Brij 96/Brij O10). In some embodiments, the unsaturated fatty ether is polyoxyethylene (10) oleyl ether (Brij 96/Brij O10).

In some embodiments, the one or more excipients are present in the formulation in an amount of from about 1% to about 10% by weight of the base formulation. In some embodiments, the one or more excipients are present in an amount of from about 2% to about 7%, from about 3% to about 7%, from about 3% to about 6%, or from about 3% to about 5% by weight of the base formulation. In some embodiments, the one or more excipients are present in an amount of from about 3% to about 6% by weight of the base formulation. In some embodiments, the one or more excipients are present in an amount of from about 3% to about 5% by weight of the base formulation.

In some embodiments, the one or more excipients are oleyl alcohol; and oleyl alcohol is present in the formulation in an amount of from about 1% to about 10% by weight of the base formulation. In some embodiments, oleyl alcohol is present in an amount of from about 1% to about 10%, from about 2% to about 7%, from about 3% to about 7%, from about 3% to about 6%, or from about 3% to about 5% by weight of the base formulation. In some embodiments, oleyl alcohol is present in an amount of from about 3% to about 6%, or from about 3% to about 5% by weight of the base formulation. In some embodiments, oleyl alcohol is present in an amount of from about 3% to about 6% by weight of the base formulation. In some embodiments, oleyl alcohol is present in an amount of from about 3% to about 5% by weight of the base formulation. In some embodiments, oleyl alcohol is present in an amount of about 5% by weight of the base formulation.

### IIIA-2. DMSO

In some embodiments, DMSO is present in the formulation. In some embodiments, DMSO is present in an amount of from about 5% to about 20%, from about 5% to about 15%, from about 10% to about 15%, or from about 5% to about 10% by weight of the base formulation. In some embodiments, DMSO is present in an amount of from about 5% to about 15%, from about 10% to about 15%, or from about 5% to about 10% by weight of the base formulation. In some embodiments, DMSO is present in an amount of from about 5% to about 15% by weight of the base formulation. In some embodiments, DMSO is present in an amount of from about 10% to about 15% by weight of the base formulation. In some embodiments, DMSO is present in an amount of from about 5% to about 10% by weight of the base formulation. In some embodiments, DMSO is present in an amount of about 5%, about 7.5%, about 10%, about 15% by weight of the base formulation, or any range therebetween. In some embodiments, DMSO is present in an amount of about 5% by weight of the base formulation. In some embodiments, DMSO is present in an amount of about 7.5% by weight of the base formulation. In some embodiments, DMSO is present in an amount of about 10% by weight of the base formulation. In some embodiments, DMSO is present in an amount of about 15% by weight of the base formulation.

In some embodiments, DMSO is absent from the formulation.

### IIIA-3. Acid

An acid included in the formulation can be any acid that is capable of protonating a hydroperoxyl species without causing additional hydrolysis of the compound of formula (I) under an acidic condition. As described herein, the hydroperoxyl species are the deprotonated species of peroxides present in one or more excipients including an unsaturated fatty alcohol (e.g., oleyl alcohol), an unsaturated fatty acid (e.g., oleic acid), or an unsaturated ester or ether (e.g., containing the sidechain of oleyl alcohol and/or oleic acid). Suitable acids include organic acids having a pKa of from about 3 to about 6 and inorganic acids as described herein.

In some embodiments, the acid is an organic acid having a pKa of from about 3 to about 6. Suitable organic acids having a pKa of from about 3 to about 6 include, but are not limited to, citric acid, formic acid, lactic acid, benzoic acid, oxalic acid, acetic acid, and propionic acid. In some embodiments, the acid is an organic acid selected from the group consisting of citric acid, formic acid, lactic acid, benzoic acid, oxalic acid, acetic acid, and propionic acid. In some embodiments, the acid is an organic acid selected from the group consisting of citric acid, formic acid, lactic acid, benzoic acid, acetic acid, and propionic acid. In some embodiments, the acid is an organic acid selected from the group consisting of citric acid, formic acid, lactic acid, and acetic acid. In some embodiments, the acid is an organic acid selected from the group consisting of citric acid and acetic acid. In some embodiments, the acid is an inorganic acid selected from the group consisting of hydrochloric acid (HCl), boric acid (H₃BO₃), sulfuric acid (H₂SO₄), carbonic acid (H₂CO₃), and phosphoric acid (H₃PO₄). In some embodiments, the acid is an inorganic acid selected from the group consisting of hydrochloric acid (HCl), boric acid (H₃BO₃), and phosphoric acid (H₃PO₄). In some embodiments, the acid is an inorganic acid selected from the group consisting of hydrochloric acid (HCl) and phosphoric acid (H₃PO₄). In some embodiments, the acid is citric acid, acetic acid, phosphoric acid, or a combination thereof. In some embodiments, the acid is citric acid, acetic acid, or phosphoric acid. In some embodiments, the acid is citric acid. In some embodiments, the acid is acetic acid. In some embodiments, the acid is phosphoric acid.

In some embodiments, the acid is citric acid and citric acid is present in the formulation in an amount of from about 0.005% to about 0.5% by weight of the base formulation. In some embodiments, citric acid is present in an amount of from about 0.01% to about 0.1% by weight of the base formulation. In some embodiments, citric acid is present in an amount of about 0.05% by weight of the base formulation.

In some embodiments, the acid is acetic acid and acetic acid is present in the formulation in an amount of from about 0.005% to about 1% by weight of the base formulation. In some embodiments, acetic acid is present in an amount of from about 0.01% to about 0.5% by weight of the base formulation. In some embodiments, acetic acid is present in an amount of from about 0.05% to about 0.3% by weight of the base formulation. In some embodiments, acetic acid is present in an amount of from about 0.05% to about 0.1% by weight of the base formulation. In some embodiments, acetic acid is present in an amount of from about 0.02% to about 0.1% by weight of the base formulation. In some embodiments, acetic acid is present in an amount of about 0.05% by weight of the base formulation.

In some embodiments, the acid is phosphoric acid and phosphoric acid is present in the formulation in an amount of from about 0.001% to about 0.03% by weight of the base formulation. In some embodiments, phosphoric acid is present in an amount of from about 0.001% to about 0.01% by weight of the base formulation. In some embodiments, phosphoric acid is present in an amount of about 0.005% by weight of the base formulation.

### IIIA-4. Solvents

In some embodiments, the one or more solvents include a C₂₋₆ alkylene glycol. In some embodiments, the one or more solvents include a di-(C₂₋₆ alkylene) glycol. In some embodiments, the one or more solvents include a C₂₋₆ alkylene glycol and a di-(C₂₋₆ alkylene) glycol. In some embodiments, the one or more solvents further include C₁₋₃ alkyl-(OCH₂CH₂)₁₋₅—OH, a polyethylene glycol, or a combination thereof. In some embodiments, the one or more solvents include C₁₋₃ alkyl-(OCH₂CH₂)₁₋₅—OH, a C₂₋₆ alkylene glycol, a di-(C₂₋₆ alkylene) glycol, a polyethylene glycol, or a combination thereof, provided that at least one of a C₂₋₆ alkylene glycol and a di-(C₂₋₆ alkylene) glycol is present. In some embodiments, the one or more solvents include a C₂₋₆ alkylene glycol; and further include C₁₋₃ alkyl-(OCH₂CH₂)₁₋₅—OH, a di-(C₂₋₆ alkylene) glycol, a polyethylene glycol, or a combination thereof.

In some embodiments, C₁₋₃ alkyl-(OCH₂CH₂)₁₋₅—OH is 2-(2-ethoxyethoxy)ethanol (i.e., Transcutol P). In some embodiments, the C₂₋₆ alkylene glycol is propylene glycol. In some embodiments, the di-(C₂₋₆ alkylene) glycol is dipropylene glycol. In some embodiments, the polyethylene glycol is PEG200, PEG300, PEG400, PEG600, PEG900, or a combination thereof. In some embodiments, the polyethylene glycol includes PEG400. In some embodiments, the polyethylene glycol is PEG400. In some embodiments, C₁₋₃ alkyl-(OCH₂CH₂)₁₋₅—OH is 2-(2-ethoxyethoxy)ethanol; the C₂₋₆ alkylene glycol is propylene glycol; the di-(C₂₋₆ alkylene) glycol is dipropylene glycol; and the polyethylene glycol is PEG400.

In some embodiments, the one or more solvents includes 2-(2-ethoxyethoxy)ethanol, propylene glycol, and dipropylene glycol. In some embodiments, the one or more solvents includes 2-(2-ethoxyethoxy)ethanol, propylene glycol, dipropylene glycol, and PEG400.

In some embodiments, 2-(2-ethoxyethoxy)ethanol is present in the formulation in an amount of from about 20% to about 40%, from about 20% to about 35%, from about 20% to about 30%, from about 25% to about 35%, from about 25% to about 30%, from about 30% to about 35%, or from about 30% to about 40% by weight of the base formulation. In some embodiments, 2-(2-ethoxyethoxy)ethanol is present in an amount of from about 20% to about 40%, from about 20% to about 35%, or from about 20% to about 30% by weight of the base formulation. In some embodiments, 2-(2-ethoxyethoxy)ethanol is present in an amount of from about 25% to about 30%, from about 30% to about 35%, or from about 30% to about 40% by weight of the base formulation. In some embodiments, 2-(2-ethoxyethoxy)ethanol is present in an amount of from about 20% to about 30% by weight of the base formulation. In some embodiments, 2-(2-ethoxyethoxy)ethanol is present in an amount of about 25% by weight of the base formulation.

In some embodiments, propylene glycol is present in the formulation in an amount of from about 30% to about 70%, from about 30% to about 60%, from about 40% to about 60%, or from about 45% to about 55% by weight of the base formulation. In some embodiments, propylene glycol is present in an amount of from about 40% to about 60% or from about 45% to about 55% by weight of the base formulation. In some embodiments, propylene glycol is present in an amount of from about 40% to about 60% by weight of the base formulation. In some embodiments, propylene glycol is present in an amount of from about 45% to about 55% by weight of the base formulation. In some embodiments, propylene glycol is present in an amount of about 50% by weight of the base formulation. In some embodiments, propylene glycol is present in an amount of about 45% by weight of the base formulation.

In some embodiments, dipropylene glycol is present in the formulation in an amount of from about 1% to about 25%, from about 1% to about 20%, or from about 1% to about 10% by weight of the base formulation. In some embodiments, dipropylene glycol is present in an amount of from about 1% to about 10% by weight of the base formulation. In some embodiments, dipropylene glycol is present in an amount of from about 2% to about 8%, from about 3% to about 7%, or from about 4% to about 6% by weight of the base formulation. In some embodiments, dipropylene glycol is present in an amount of from about 3% to about 7% or from about 4% to about 6% by weight of the base formulation. In some embodiments, dipropylene glycol is present in an amount of from about 4% to about 6% by weight of the base formulation. In some embodiments, dipropylene glycol is present in an amount of about 5% by weight of the base formulation.

In some embodiments, PEG400 is present in the formulation in an amount of from about 5% to about 50%, from about 5% to about 40%, from about 5% to about 30%, from about 5% to about 20%, or from about 5% to about 15% by weight of the base formulation. In some embodiments, PEG400 is present in an amount of from about 5% to about 30%, from about 5% to about 20%, or from about 5% to about 15% by weight of the base formulation. In some embodiments, when DMSO is present in the formulation, PEG400 is present in an amount of from about 5% to about 20% or from about 5% to about 15% by weight of the base formulation. In some embodiments, when DMSO is present, PEG400 is present in an amount of from about 5% to about 15% by weight of the base formulation. In some embodiments, when DMSO is present, PEG400 is present in an amount of about 10% by weight of the base formulation. In some embodiments, when DMSO is absent from the formulation, PEG400 is present in an amount of from about 15% to about 30% by weight of the base formulation. In some embodiments, when DMSO is absent, PEG400 is present in an amount of from about 20% to about 25% by weight of the base formulation. In some embodiments, when DMSO is absent, PEG400 is present in an amount of about 22% by weight of the base formulation.

### IIIA-5. Viscosity

A formulation as provided herein can have a low viscosity (e.g., about 1 cP to 5,000 cP) when a gelling agent (e.g., a polymer thickener) is absent, a gelling agent having a low molecular weight is present in a suitable amount, or a gelling agent having a medium or high average molecular weight is present in a suitable amount (e.g., <0.5 by weight of the base formulation).

In some embodiments, a gelling agent is absent in the formulation. In some embodiments, the formulation has a viscosity of from about 1 cP to about 1,000 cP. In some embodiments, the formulation has a viscosity of from about 50 cP to about 1,000 cP, from about 50 cP to about 900 cP, from about 50 cP to about 800 cP, from about 50 cP to about 700 cP, from about 50 cP to about 600 cP, from about 50 cP to about 500 cP, from about 50 cP to about 400 cP, from about 50 cP to about 300 cP, from about 50 cP to about 200 cP, or any suitable range therein.

When a gelling agent is included in the formulation, the formulation can have a low viscosity, e.g., from about 1,000 cP to about 5,000 cP. The low viscosity of the formulation can be achieved by using a gelling agent having a low average molecular weight in a suitable amount. Examples of the gelling agents having a low average molecular weight (e.g., an average molecular weight of 100,000 Da or less) are described in Section IIIA-6. Gelling Agent. In some embodiments, the formulation has a viscosity of from about 1,000 cP to about 5,000 cP. In some embodiments, the formulation has a viscosity of from about 1,000 cP to about 4,000 cP, from about 1,000 cP to about 3,000 cP, from about 1,000 cP to about 2,000 cP, or any suitable range therein.

When the gelling agent is present in the formulation, the formulation can have a viscosity of from about 5,000 cP to about 100,000 cP, as described in Section IIIA-6. Gelling Agent.

### IIIA-6. Gelling Agent

In some embodiments, the formulation further includes a gelling agent (e.g., a polymer thickener). Gelling agents include, for example, hydrophilic and hydroalcoholic gelling agents frequently used in the cosmetic and pharmaceutical industries. In some embodiments, the gelling agent is a Carbopol (also referred to as a carbomer), carboxymethyl cellulose, ethylcellulose, gelatin, hydroxyethyl cellulose, hydroxypropyl cellulose, magnesium aluminum silicate (Veegum), methylcellulose, a poloxamer (Pluronics), polyvinyl alcohol, sodium alginate, tragacanth, xanthan gum, or a combination thereof. In some embodiments, the gelling agent includes hydroxypropyl cellulose. In some embodiments, the gelling agent is hydroxypropyl cellulose.

In some embodiments, the hydroxypropyl cellulose has an average molecular weight selected from the group consisting of about 40,000 Dalton (Da), about 80,000 Da, about 100,000 Da, about 140,000 Da, about 180,000 Da, about 280,000 Da, about 370,000 Da, about 700,000 Da, about 850,000 Da, about 1,000,000 Da, about 1,150,000 Da, and about 2,500,000 Da. In some embodiments, the hydroxypropyl cellulose has the average molecular weight selected from the group consisting of about 140,000 Da, about 180,000 Da, about 280,000 Da, about 370,000 Da, about 700,000 Da, about 850,000 Da, about 1,000,000 Da, and about 1,150,000 Da. In some embodiments, the hydroxypropyl cellulose has the average molecular weight selected from the group consisting of about 700,000 Da, about 850,000 Da, about 1,000,000 Da, and about 1,150,000 Da.

Hydroxypropyl cellulose (HPC) includes, for example Nisso SSL, Nisso SL, Nisso L, Nisso LM, Nisso LMM, Nisso M, Nisso H, Nisso VH, Klucel ELF, Klucel EF, Klucel LF, Klucel JF, Klucel GF, Klucel MF, and Klucel HF.

Nisso SSL has an average molecular weight of about 40,000 Da; Nisso SL has an average molecular weight of about 100,000 Da; Nisso L has an average molecular weight of about 140,000 Da; Nisso LM has an average molecular weight of about 180,000 Da; Nisso LMM has an average molecular weight of about 280,000 Da; Nisso M has an average molecular weight of about 700,000 Da; Nisso H has an average molecular weight of about 1,000,000 Da; and Nisso VH has an average molecular weight of about 2,500,000 Da. Suitable particle sizes of Nisso HPC (i.e., Nisso SSL, Nisso SL, Nisso L, Nisso LM, Nisso LMM, Nisso M, Nisso H, and Nisso VH) in the formulation include regular powder (40 mesh), fine powder (100 mesh), and super fine powder (300 mesh). See Technical date sheets of Nisso HPCs, the entirety of which is incorporated herein by reference for all purposes. In some embodiments, the hydroxypropyl cellulose is Nisso H.

Klucel ELF has an average molecular weight of about 40,000 Da; Klucel EF has an average molecular weight of about 80,000 Da; Klucel LF has an average molecular weight of about 95,000 Da; Klucel JF has an average molecular weight of about 140,000 Da; Klucel GF has an average molecular weight of about 370,000 Da; Klucel MF has an average molecular weight of about 850,000 Da; and Klucel HF has an average molecular weight of about 1,150,000 Da. Suitable particle sizes of Klucel HPC in the formulation include regular grade and fine grade. See Technical date sheets of Klucel HPC products, the entirety of which is incorporated herein by reference for all purposes. In some embodiments, the hydroxypropyl cellulose is Klucel HF.

When the gelling agent is present in the formulation, in some embodiments, the formulation has a viscosity of from about 5,000 cP to about 100,000 cP. When the gelling agent is present, in some embodiments, the formulation has a viscosity of from about 5,000 cP to about 50,000 cP. When the gelling agent is present, in some embodiments, the formulation has a viscosity of from about 5,000 cP to about 15,000 cP. When the gelling agent is present, in some embodiments, the formulation has a viscosity of from about 10,000 cP to about 20,000 cP. When the hydroxypropyl cellulose is present, in some embodiments, the formulation has a viscosity of from about 5,000 cP to about 100,000 cP. When the hydroxypropyl cellulose is present, in some embodiments, the formulation has a viscosity of from about 5,000 cP to about 50.000 cP. When the hydroxypropyl cellulose is present, in some embodiments, the formulation has a viscosity of from about 5,000 cP to about 15,000 cP. When the hydroxypropyl cellulose is present, in some embodiments, the formulation has a viscosity of from about 10,000 cP to about 20.000 cP.

In some embodiments, the gelling agent is present in the formulation in an amount of from about 0.5% to about 30% by weight of the base formulation, while the formulation has a viscosity of from about 5,000 cP to about 100,000 cP. In some embodiments, the gelling agent is present in an amount of from about 0.5% to about 30% by weight of the base formulation, while the formulation has a viscosity of from about 5,000 cP to about 50,000 cP. In some embodiments, the gelling agents are present in an amount of from about 0.5% to about 30% by weight of the base formulation, while the formulation has a viscosity of from about 5,000 cP to about 20,000 cP. In some embodiments, the gelling agents are present in an amount of from about 0.5% to about 30% by weight of the base formulation, while the formulation has a viscosity of from about 5,000 cP to about 15,000 cP. In some embodiments, the gelling agents are present in an amount of from about 0.5% to about 30% by weight of the base formulation, while the formulation has a viscosity of from about 10,000 cP to about 20,000 cP. In some embodiments, the hydroxypropyl cellulose is present in an amount of from about 0.5% to about 5%, from about 5% to about 10%, from about 10% to about 20%, or from about 20% to about 30% by weight of the base formulation, while the formulation has a viscosity of from about 5.000 cP to about 100,000 cP. When a hydroxypropyl cellulose having an average molecular weight of less than about 700,000 Da is used, in some embodiments, the hydroxypropyl cellulose is present in an amount of about 5% to about 30% by weight of the base formulation, while the formulation has a viscosity of from about 5,000 cP to about 100,000 cP. In some embodiments, the hydroxypropyl cellulose is present in an amount of from about 0.5% to about 4%, from about 0.5% to about 3%, from about 0.5% to about 2%, from about 1% to about 5%, from about 1% to about 4%, from about 1% to about 3%, from about 1% to about 2%, or from about 2% to about 5% by weight of the base formulation, while the formulation has a viscosity of from about 5,000 cP to about 20,000 cP. In some embodiments, the hydroxypropyl cellulose is present in an amount of from about 0.5% to about 4%, from about 0.5% to about 3%, from about 0.5% to about 2%, from about 1% to about 5%, from about 1% to about 4%, from about 1% to about 3%, from about 1% to about 2%, or from about 2% to about 5% by weight of the base formulation, while the formulation has a viscosity of from about 5,000 cP to about 15,000 cP. In some embodiments, the hydroxypropyl cellulose is present in an amount of from about 0.5% to about 4%, from about 0.5% to about 3%, from about 0.5% to about 2%, from about 1% to about 5%, from about 1% to about 4%, from about 1% to about 3%, from about 1% to about 2%, or from about 2% to about 5% by weight of the base formulation, while the formulation has a viscosity of from about 10,000 cP to about 20,000 cP. In some embodiments, the hydroxypropyl cellulose is present in an amount of from about 1% to about 5%, from about 1% to about 4%, or from about 1% to about 3% by weight of the base formulation, while the formulation has a viscosity of from about 5,000 cP to 20,000 cP. In some embodiments, the hydroxypropyl cellulose is present in an amount of from about 1% to about 5%, from about 1% to about 4%, or from about 1% to about 3% by weight of the base formulation, while the formulation has a viscosity of from about 5,000 cP to 15,000 cP. In some embodiments, the hydroxypropyl cellulose is present in an amount of from about 1% to about 5%, from about 1% to about 4%, or from about 1% to about 3% by weight of the base formulation, while the formulation has a viscosity of from about 10,000 cP to 20.000 cP. In some embodiments, the hydroxypropyl cellulose is present in an amount of from about 0.5% to about 2% by weight of the base formulation, while the formulation has a viscosity of from about 5,000 cP to about 20,000 cP. In some embodiments, the hydroxypropyl cellulose is present in an amount of from about 0.5% to about 2% by weight of the base formulation, while the formulation has a viscosity of from about 5,000 cP to about 15,000 cP. In some embodiments, the hydroxypropyl cellulose is present in an amount of from about 0.5% to about 2% by weight of the base formulation, while the formulation has a viscosity of from about 10,000 cP to about 20,000 cP.

In some embodiments, the hydroxypropyl cellulose having an average molecular weight of from about 700,000 Da to about 1,150,000 Da is present in an amount of from about 0.5% to about 2% by weight of the base formulation. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 20,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 15,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 10,000 cP to about 20,000 cP.

In some embodiments, the hydroxypropyl cellulose having an average molecular weight of from about 700,000 Da to about 1,150,000 Da is present in an amount of about 1% by weight of the base formulation. In some embodiments, the hydroxypropyl cellulose having an average molecular weight of from about 700,000 Da to about 1,150,000 Da is present in an amount of about 2% by weight of the base formulation.

### IIIA-7. Compounds of Formula (I)

The compound of formular (I), a hydrate, a solvate, a pharmaceutically acceptable salt, or a combination thereof, is further described according to Section V. COMPOUNDS.

In some embodiments, the compound of formula (I) in any one of the formulations is represented by formula (Ha):

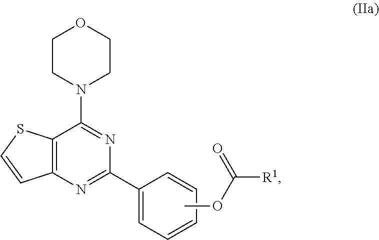

wherein R¹ is as defined and described herein.

In some embodiments, the compound of formula (I) in any one the formulations is represented by any one of formulae (IIb), (IIc), and (IId):

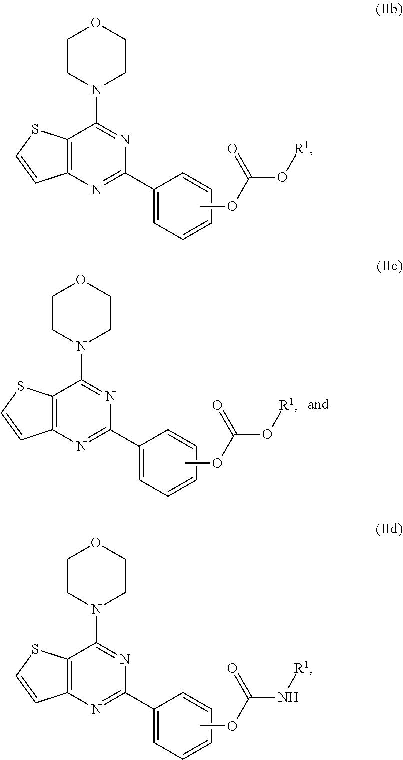

wherein R¹ is as defined herein in any aspect or embodiments described herein.

In some embodiments, the compound of formula (I) in any one of the formulations is represented by formula (IIa-1a):

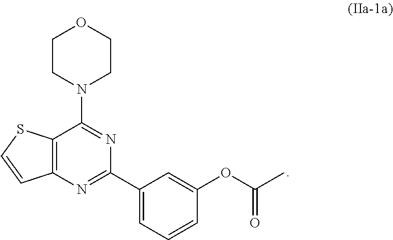 [SMILES: CC(=O)Oc1cccc(-c2nc(N3CCOCC3)c3sccc3n2)c1]

In some embodiments, the compound of formula (I) in any one of the formulations is represented by formula (IIIa):

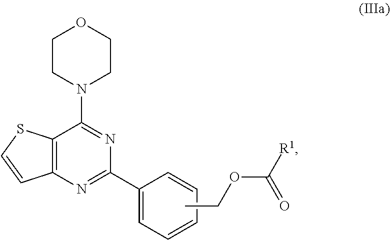

wherein R¹ is as defined herein in any aspect or embodiments described herein.

In some embodiments, the compound of formula (I) in any one of the formulations is represented by any one of formulae (IIIb), (IIIc), and (IIId):

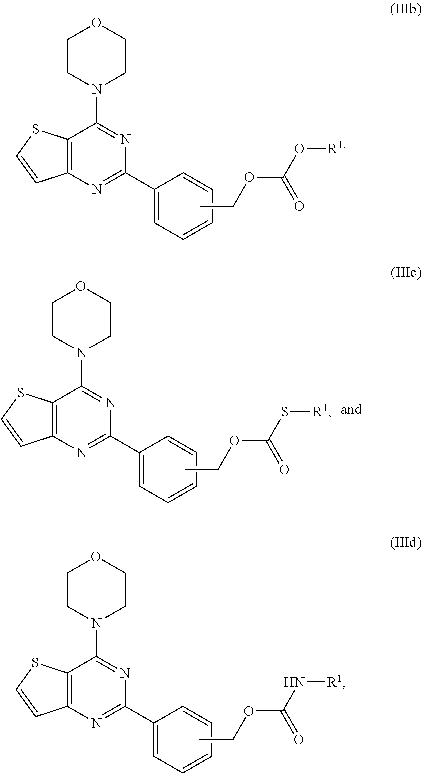

wherein R¹ is as defined herein in any aspect or embodiments described herein.

In some embodiments, the compound of formula (I) in any one of the formulations is represented by formula (IIIa-1a):

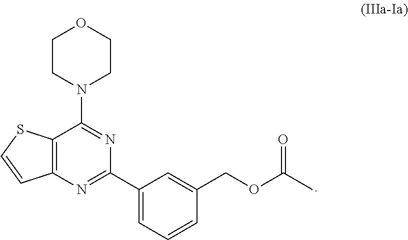 [SMILES: CC(=O)OCc1cccc(-c2nc(N3CCOCC3)c3sccc3n2)c1]

In some embodiments, the compound of formula (I) or a pharmaceutically acceptable salt thereof is in an anhydrous form. In some embodiments, the compound of formula (I) or a pharmaceutically acceptable salt thereof is in a hydrous form. In some embodiments, the compound of formula (I) or a pharmaceutically acceptable salt thereof is a mixture of an anhydrous and hydrate forms.

In some embodiments, the compound of formula (IIa-1) or a pharmaceutically acceptable salt thereof is in an anhydrous form. In some embodiments, the compound of formula (IIa-1) or a pharmaceutically acceptable salt thereof is in a hydrous form. In some embodiments, the compound of formula (IIa-1) or a pharmaceutically acceptable salt thereof is a mixture of an anhydrous and hydrate forms.

In some embodiments, the compound of formula (I) used in preparing the formulation is in a salt-free form. In some embodiments, the compound of formula (IIa-1) used in preparing the formulation is in a salt-free form.

In some embodiments of any one of formulations, the compound of formula (I) is present in the formulation in a degree to saturation of from about 50% to about 100%. In some embodiments, the compound of formula (I) is present in a degree to saturation of from about 75% to about 100%. In some embodiments, the compound of formula (I) is present in a degree to saturation of from about 80% to about 100%, from about 90% to about 100%, or about 100%. In some embodiments, the compound of formula (I) is present in a degree to saturation of from about 90% to about 100%. In some embodiments, the compound of formula (I) is present at a saturated concentration in the formulation (i.e., a degree to saturation of about 100%).

In some embodiments of any one of formulations, the compound of formula (IIa-1) is present in the formulation in a degree to saturation of from about 50% to about 100%. In some embodiments, the compound of formula (IIa-1) is present in a degree to saturation of from about 75% to about 100%. In some embodiments, the compound of formula (IIa-1) is present in a degree to saturation of from about 80% to about 100%, from about 90% to about 100%, or about 100%. In some embodiments, the compound of formula (IIa-1) is present in a degree to saturation of from about 90% to about 100%. In some embodiments, the compound of formula (IIa-1) is present at a saturated concentration in the formulation (i.e., a degree to saturation of about 100%).

In some embodiments of any one of formulations, the compound of formula (I) is present in an amount of from about 0.05% to about 15%, from about 0.5% to about 12%, from about 0.5% to about 10%, from about 1% to about 10%, from about 2% to about 10%, from about 5% to about 10%, from about 2% to 5%, or from about 3% to 5% by weight of the base formulation on a salt-free and anhydrous basis. In some embodiments, the compound of formula (I) is present in an amount of from about 3% to 5% by weight of the base formulation on a salt-free and anhydrous basis. In some embodiments, the compound of formula (I) is present in an amount of about 2% by weight of the base formulation on a salt-free and anhydrous basis. In some embodiments, the compound of formula (I) is present in an amount of about 3% by weight of the base formulation on a salt-free and anhydrous basis. In some embodiments, the compound of formula (I) is present in an amount of about 4% by weight of the base formulation on a salt free and anhydrous basis. In some embodiments, the compound of formula (I) is present in an amount of about 5% by weight of the base formulation on a salt free and anhydrous basis.

In some embodiments of any one of formulations, the compound of formula (IIa-1) is present in the formulation in an amount of from about 0.05% to about 15%, from about 0.5% to about 12%, from about 0.5% to about 10%, from about 1% to about 10%, from about 2% to about 10%, from about 5% to about 10%, from about 2% to about 5%, or from about 3% to 5% by weight of the base formulation on a salt-free and anhydrous basis. In some embodiments, the compound of formula (IIa-1) is present in an amount of from about 3% to 5% by weight of the base formulation on a salt-free and anhydrous basis. In some embodiments, the compound of formula (IIa-1) is present in an amount of about 2% by weight of the base formulation on a salt-free and anhydrous basis. In some embodiments, the compound of formula (IIa-1) is present in an amount of about 3% by weight of the base formulation on a salt-free and anhydrous basis. In some embodiments, the compound of formula (IIa-1) is present in an amount of about 4% by weight of the base formulation on a salt free and anhydrous basis. In some embodiments, the compound of formula (IIa-1) is present in an amount of about 5% by weight of the base formulation on a salt free and anhydrous basis.

### IIIA-8. Apparent pH Value

The formulation as described herein is an non-aqueous formulations, thereof the pH value of the formulation is an apparent pH value. According to US Pharmacopeia (USP) chapter <791>, the apparent pH value of a non-aqueous solution or suspension is anticipated for variability, which may be up to approximately 1 pH unit. See USP chapter <791>, the entirety of which is incorporated herein by reference for all purposes.

In some embodiments, the formulation has an apparent pH value of from about 4 to about 6. In some embodiments, the formulation has an apparent pH value of from about 4 to about 5.

### IIIA-9. Selected Embodiments

In some embodiments, the formulation (AA) includes:
a) the compound of formula (I);
b) a base formulation including:
   b1) DMSO in an amount of from about 1% to about 10% by weight of the base formulation;
   b2) the unsaturated fatty alcohol in an amount of from about 1% to about 10% by weight of the base formulation; and
   b3) C₁₋₃ alkyl-(OCH₂CH₂)₁₋₅—OH, the C₂₋₆ alkylene glycol, the di-(C₂₋₆ alkylene) glycol, and the polyethylene glycol;
c) an acid; and
d) the gelling agent,
   wherein:
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
C₁₋₃ alkyl-(OCH₂CH₂)₁₋₅—OH, the C₂₋₆ alkylene glycol, the di-(C₂₋₆ alkylene) glycol, the polyethylene glycol, the unsaturated fatty alcohol, and the gelling agent are as described herein.

In some embodiments of formulation (AA), the unsaturated fatty alcohol is oleyl alcohol. In some embodiments, oleyl alcohol is present in the formulation in an amount of from about 1% to about 10%, from about 2% to about 10%, from about 2% to about 8%, from about 2% to about 6%, from about 3% to about 6%, from about 4% to about 6%, from about 2% to about 5%, from about 2% to about 4%, or from about 3% to about 5% by weight of the base formulation. In some embodiments, oleyl alcohol is present in an amount of from about 4% to about 6%, from about 2% to about 4%, or from about 3% to about 5% by weight of the base formulation. In some embodiments, oleyl alcohol is present in an amount of from about 4% to about 6% by weight of the base formulation. In some embodiments, oleyl alcohol is present in an amount of about 2%, about 3%, about 4%, or about 5% by weight of the base formulation. In some embodiments, oleyl alcohol is present in an amount of about 3% by weight of the base formulation. In some embodiments, oleyl alcohol is present in an amount of about 4% by weight of the base formulation. In some embodiments, oleyl alcohol is present in an amount of about 5% by weight of the base formulation.

In some embodiments of formulation (AA), DMSO, C₁₋₃ alkyl-(OCH₂CH₂)₁₋₅—OH, and the polyethylene glycol are present in the formulation in a total amount of from about 35% to about 45% by weight of the base formulation. In some embodiments, DMSO, C₁₋₃ alkyl-(OCH₂CH₂)₁₋₅—OH, and the polyethylene glycol are present in a total amount of about 40% by weight of the base formulation. In some embodiments, DMSO, 2-(2-ethoxyethoxy)ethanol, and PEG400 are present in a total amount of from about 35% to about 45% by weight of the base formulation. In some embodiments, DMSO, 2-(2-ethoxyethoxy)ethanol, and PEG400 are present in a total amount of about 40% by weight of the base formulation.

In some embodiments of formulation (AA), DMSO is present in the formulation in an amount of from about 3% to about 7% by weight of the base formulation. In some embodiments, DMSO is present in an amount of about 5% by weight of the base formulation.

In some embodiments of formulation (AA), C₁₋₃ alkyl-(OCH₂CH₂)₁₋₅—OH is 2-(2-ethoxyethoxy)ethanol present in the formulation in an amount of from about 20% to about 40%, from about 20% to about 35%, or from about 20% to about 30% by weight of the base formulation. In some embodiments, 2-(2-ethoxyethoxy)ethanol is present in an amount of from about 20% to about 30% by weight of the base formulation. In some embodiments, 2-(2-ethoxyethoxy)ethanol is present in an amount of about 25% by weight of the base formulation.

In some embodiments of formulation (AA), the C₂₋₆ alkylene glycol is propylene glycol present in the formulation in an amount of from about 30% to about 70%, from about 30% to about 60%, from about 40% to about 60%, or from about 45% to about 55% by weight of the base formulation. In some embodiments, propylene glycol is present in an amount of from about 40% to about 60% or from about 45% to about 55% by weight of the base formulation. In some embodiments, propylene glycol is present in an amount of from about 40% to about 60% by weight of the base formulation. In some embodiments, propylene glycol is present in an amount of from about 45% to about 55% by weight of the base formulation. In some embodiments, propylene glycol is present in an amount of about 50% by weight of the base formulation. In some embodiments, propylene glycol is present in an amount of about 52% by weight of the base formulation.

In some embodiments of formulation (AA), the di-(C₂₋₆ alkylene) glycol is dipropylene glycol present in the formulation in an amount of from about 1% to about 10%, from about 2% to about 8%, from about 3% to about 7%, or from about 4% to about 6% by weight of the base formulation. In some embodiments, dipropylene glycol is present in an amount of from about 3% to about 7% or from about 4% to about 6% by weight of the base formulation. In some embodiments, dipropylene glycol is present in an amount of from about 4% to about 6% by weight of the base formulation. In some embodiments, dipropylene glycol is present in an amount of about 5% by weight of the base formulation.

In some embodiments of formulation (AA), the polyethylene glycol is PEG400 present in the formulation in an amount of from about 5% to about 15% by weight of the base formulation. In some embodiments, PEG400 is present in an amount of from about 5% to about 15% by weight of the base formulation. In some embodiments, PEG400 is present in an amount of about 10% by weight of the base formulation.

In some embodiments of formulation (AA), the gelling agent is hydroxypropyl cellulose. In some embodiments, hydroxypropyl cellulose is present in an amount of from about 0.5% to about 2% by weight of the base formulation, while the formulation has a viscosity of from about 5,000 cP to about 20,000 cP. In some embodiments, hydroxypropyl cellulose is present in an amount of from about 0.5% to about 2% by weight of the base formulation, while the formulation has a viscosity of from about 10,000 cP to about 20,000 cP. In some embodiments, the hydroxypropyl cellulose having an average molecular weight of from about 700,000 Da to about 1,150,000 Da is present in an amount of from 0.5% to about 2% by weight of the base formulation. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 20,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 10,000 cP to about 20,000 cP.

In some embodiments of formulation (AA), the acid is citric acid and citric acid is present in the formulation in an amount of from about 0.01% to about 0.1% by weight of the base formulation. In some embodiments, citric acid is present in an amount of about 0.05% by weight of the base formulation.

In some embodiments of formulation (AA), the acid is acetic acid and acetic acid is present in the formulation in an amount of from about 0.01% to about 0.5% by weight of the base formulation. In some embodiments, acetic acid is present in an amount of about 0.05% by weight of the base formulation.

In some embodiments of formulation (AA), the acid is phosphoric acid and phosphoric acid is present in the formulation in an amount of from about 0.001% to about 0.01% by weight of the base formulation. In some embodiments, phosphoric acid is present in an amount of about 0.005% by weight of the base formulation.

In some embodiments, the formulation (AA1ca) includes:
a) the compound of formula (I);
b) a base formulation including:
   b1) DMSO in an amount of about 5% by weight of the base formulation;
   b2) oleyl alcohol in an amount of about 5% by weight of the base formulation; and
   b3) 2-(2-ethoxyethoxy)ethanol, propylene glycol, dipropylene glycol, and PEG400, which are present in an amount of about 25%, about 50%, about 5%, and about 10%, respectively, by weight of the base formulation;
c) citric acid in an amount of about 0.05% by weight of the base formulation; and
d) hydroxypropyl cellulose in an amount of from about 0.5% to about 2% by weight of the base formulation.

In some embodiments, the formulation (AA2ca) includes:
a) the compound of formula (I);
b) a base formulation including:
   b1) DMSO in an amount of about 5% by weight of the base formulation;
   b2) oleyl alcohol in an amount of about 3% by weight of the base formulation; and
   b3) 2-(2-ethoxyethoxy)ethanol, propylene glycol, dipropylene glycol, and PEG400, which are present in an amount of about 25%, about 52%, about 5%, and about 10%, respectively, by weight of the base formulation;
c) citric acid in an amount of about 0.05% by weight of the base formulation; and
d) hydroxypropyl cellulose in an amount of from about 0.5% to about 2% by weight of the base formulation.

In some embodiments, the formulation (AA1aa) includes:
a) the compound of formula (I);
b) a base formulation including:
   b1) DMSO in an amount of about 5% by weight of the base formulation;
   b2) oleyl alcohol in an amount of about 5% by weight of the base formulation;
   b3) 2-(2-ethoxyethoxy)ethanol, propylene glycol, dipropylene glycol, and PEG400, which are present in an amount of about 25%, about 50%, about 5%, and about 10%, respectively, by weight of the base formulation;
c) acetic acid in an amount of about 0.05% by weight of the base formulation; and
d) hydroxypropyl cellulose in an amount of from about 0.5% to about 2% by weight of the base formulation.

In some embodiments, the formulation (AA2aa) includes:
a) the compound of formula (I);
b) a base formulation including:
   b1) DMSO in an amount of about 5% by weight of the base formulation;
   b2) oleyl alcohol in an amount of about 3% by weight of the base formulation; and
   b3) 2-(2-ethoxyethoxy)ethanol, propylene glycol, dipropylene glycol, and PEG400, which are present in an amount of about 25%, about 52%, about 5%, and about 10%, respectively, by weight of the base formulation;
c) acetic acid in an amount of about 0.05% by weight of the base formulation; and
d) hydroxypropyl cellulose in an amount of from about 0.5% to about 2% by weight of the base formulation.

In some embodiments, the formulation (AA1pa) includes:
a) the compound of formula (I);
b) a base formulation including:
   b1) DMSO in an amount of about 5% by weight of the base formulation;
   b2) oleyl alcohol in an amount of about 5% by weight of the base formulation; and
   b3) 2-(2-ethoxyethoxy)ethanol, propylene glycol, dipropylene glycol, and PEG400, which are present in an amount of about 25%, about 50%, about 5%, and about 10%, respectively, by weight of the base formulation;
c) phosphoric acid in an amount of about 0.005% by weight of the base formulation; and
d) hydroxypropyl cellulose in an amount of from 0.5% to about 2% by weight of the base formulation.

In some embodiments, the formulation (AA2pa) includes:
a) the compound of formula (I);
b) a base formulation including:
   b1) DMSO in an amount of about 5% by weight of the base formulation;
   b2) oleyl alcohol in an amount of about 3% by weight of the base formulation; and
   b3) 2-(2-ethoxyethoxy)ethanol, propylene glycol, dipropylene glycol, and PEG400, which are present in an amount of about 25%, about 52%, about 5%, and about 10%, respectively, by weight of the base formulation;
c) phosphoric acid in an amount of about 0.005% by weight of the base formulation; and
d) hydroxypropyl cellulose in an amount of from 0.5% to about 2% by weight of the base formulation.

In some embodiments of any one of formulations (AA1ca), (AA2ca), (AA1aa), (AA2aa), (AA1pa), and (AA2pa), the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

In some embodiments, the formulation (AB) includes:
a) the compound of formula (I);
b) a base formulation including:
   b1) DMSO in an amount of from about 5% to about 17% by weight of the base formulation;
   b2) the unsaturated fatty alcohol in an amount of from about 1% to about 10% by weight of the base formulation; and
   b3) C₁₋₃ alkyl-(OCH₂CH₂)₁₋₅—OH, the C₂₋₆ alkylene glycol, and the di-(C₂₋₆ alkylene) glycol;
c) an acid; and
d) the gelling agent,
   wherein:
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
C₁₋₃ alkyl-(OCH₂CH₂)₁₋₅—OH, the C₂₋₆ alkylene glycol, the di-(C₂₋₆ alkylene) glycol, the unsaturated fatty alcohol, and the gelling agent are as described herein.

In some embodiments of formulation (AB), the unsaturated fatty alcohol is oleyl alcohol. In some embodiments, oleyl alcohol is present in the formulation in an amount of from about 1% to about 10%, from about 2% to about 10%, from about 2% to about 8%, from about 2% to about 6%, from about 3% to about 6%, from about 4% to about 6%, from about 2% to about 5%, from about 2% to about 4%, or from about 3% to about 5% by weight of the base formulation. In some embodiments, oleyl alcohol is present in an amount of from about 4% to about 6%, from about 2% to about 4%, or from about 3% to about 5% by weight of the base formulation. In some embodiments, oleyl alcohol is present in an amount of from about 4% to about 6% by weight of the base formulation. In some embodiments, oleyl alcohol is present in an amount of about 2%, about 3%, about 4%, or about 5% by weight of the base formulation. In some embodiments, oleyl alcohol is present in an amount of about 3% by weight of the base formulation. In some embodiments, oleyl alcohol is present in an amount of about 4% by weight of the base formulation. In some embodiments, oleyl alcohol is present in an amount of about 5% by weight of the base formulation.

In some embodiments of formulation (AB), DMSO and C₁₋₃ alkyl-(OCH₂CH₂)₁₋₅—OH are present in the formulation in a total amount of from about 35% to about 45% by weight of the base formulation. In some embodiments, DMSO and C₁₋₃ alkyl-(OCH₂CH₂)₁₋₅—OH are present in a total amount of about 40% by weight of the base formulation. In some embodiments, DMSO and 2-(2-ethoxyethoxy)ethanol are present in a total amount of from about 35% to about 45% by weight of the base formulation. In some embodiments, DMSO and 2-(2-ethoxyethoxy)ethanol are present in a total amount of about 40% by weight of the base formulation.

In some embodiments of formulation (AB), DMSO is present in the formulation in an amount of from about 5% to about 15% by weight of the base formulation. In some embodiments, DMSO is present in an amount of about 7.5%, about 10%, or about 15% by weight of the base formulation. In some embodiments, DMSO is present in an amount of about 15% by weight of the base formulation.

In some embodiments of formulation (AB), C₁₋₃ alkyl-(OCH₂CH₂)₁₋₅—OH is 2-(2-ethoxyethoxy)ethanol present in the formulation in an amount of from about 20% to about 40%, from about 20% to about 35%, from about 20% to about 30%, from about 25% to about 35%, from about 25% to about 30%, from about 30% to about 35%, or from about 30% to about 40% by weight of the base formulation. In some embodiments, 2-(2-ethoxyethoxy)ethanol is present in an amount of about 34.5%, about 33.5%, about 32%, about 31%, about 27%, or about 25% by weight of the base formulation. In some embodiments, 2-(2-ethoxyethoxy)ethanol is present in an amount of about 34.5% by weight of the base formulation. In some embodiments, 2-(2-ethoxyethoxy)ethanol is present in an amount of about 33.5% by weight of the base formulation. In some embodiments, 2-(2-ethoxyethoxy)ethanol is present in an amount of about 32% by weight of the base formulation. In some embodiments, 2-(2-ethoxyethoxy)ethanol is present in an amount of about 31% by weight of the base formulation. In some embodiments, 2-(2-ethoxyethoxy)ethanol is present in an amount of about 27% by weight of the base formulation. In some embodiments, 2-(2-ethoxyethoxy)ethanol is present in an amount of about 25% by weight of the base formulation.

In some embodiments of formulation (AB), the C₂₋₆ alkylene glycol is propylene glycol present in the formulation in an amount of from about 30% to about 70%, from about 30% to about 60%, from about 40% to about 60%, or from about 45% to about 55% by weight of the base formulation. In some embodiments, propylene glycol is present in an amount of from about 40% to about 60% or from about 45% to about 55% by weight of the base formulation. In some embodiments, propylene glycol is present in an amount of from about 40% to about 60% by weight of the base formulation. In some embodiments, propylene glycol is present in an amount of from about 45% to about 55% by weight of the base formulation. In some embodiments, propylene glycol is present in an amount of about 50% by weight of the base formulation.

In some embodiments of formulation (AB), the di-(C₂₋₆ alkylene) glycol is dipropylene glycol present in the formulation in an amount of from about 1% to about 10%, from about 2% to about 8%, from about 3% to about 7%, or from about 4% to about 6% by weight of the base formulation. In some embodiments, dipropylene glycol is present in an amount of from about 3% to about 7% or from about 4% to about 6% by weight of the base formulation. In some embodiments, dipropylene glycol is present in an amount of from about 4% to about 6% by weight of the base formulation. In some embodiments, dipropylene glycol is present in an amount of about 5% by weight of the base formulation.

In some embodiments of formulation (AB), the gelling agent is hydroxypropyl cellulose. In some embodiments, hydroxypropyl cellulose is present in an amount of from about 0.5% to about 2% by weight of the base formulation, while the formulation has a viscosity of from about 5,000 cP to about 20,000 cP. In some embodiments, hydroxypropyl cellulose is present in an amount of from about 0.5% to about 2% by weight of the base formulation, while the formulation has a viscosity of from about 10,000 cP to about 20,000 cP. In some embodiments, the hydroxypropyl cellulose having an average molecular weight of from about 700,000 Da to about 1,150,000 Da is present in an amount of from about 0.5% to about 2% by weight of the base formulation. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 20,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 10,000 cP to about 20,000 cP.

In some embodiments of formulation (AB), the acid is citric acid and citric acid is present in the formulation in an amount of from about 0.01% to about 0.1% by weight of the base formulation. In some embodiments, citric acid is present in an amount of about 0.05% by weight of the base formulation.

In some embodiments of formulation (AB), the acid is acetic acid and acetic acid is present in the formulation in an amount of from about 0.01% to about 0.5% by weight of the base formulation. In some embodiments, acetic acid is present in an amount of about 0.05% by weight of the base formulation.

In some embodiments of formulation (AB), the acid is phosphoric acid and phosphoric acid is present in the formulation in an amount of from about 0.001% to about 0.01% by weight of the base formulation. In some embodiments, phosphoric acid is present in an amount of about 0.005% by weight of the base formulation.

In some embodiments, the formulation (AB1ca) includes:
a) the compound of formula (I);
b) a base formulation including:
   b1) DMSO in an amount of about 15% by weight of the base formulation;
   b2) oleyl alcohol in an amount of about 5% by weight of the base formulation; and
   b3) 2-(2-ethoxyethoxy)ethanol, propylene glycol, and dipropylene glycol, which are present in an amount of about 25%, about 50%, and about 5%, respectively, by weight of the base formulation;
c) citric acid in an amount of about 0.05% by weight of the base formulation; and
d) hydroxypropyl cellulose in an amount of from about 0.5% to about 2% by weight of the base formulation.

In some embodiments, the formulation (AB1aa) includes:
a) the compound of formula (I);
b) a base formulation including:
   b1) DMSO in an amount of about 15% by weight of the base formulation;
   b2) oleyl alcohol in an amount of about 5% by weight of the base formulation; and
   b3) 2-(2-ethoxyethoxy)ethanol, propylene glycol, and dipropylene glycol, which are present in an amount of about 25%, about 50%, and about 5%, respectively, by weight of the base formulation;
c) acetic acid in an amount of about 0.05% by weight of the base formulation; and
d) hydroxypropyl cellulose in an amount of from 0.5% to about 2% by weight of the base formulation.

In some embodiments, the formulation (AB1pa) includes:
a) the compound of formula (I);
b) a base formulation including:
   b1) DMSO in an amount of about 15% by weight of the base formulation;
   b2) oleyl alcohol in an amount of about 5% by weight of the base formulation; and
   b3) 2-(2-ethoxyethoxy)ethanol, propylene glycol, and dipropylene glycol, which are present in an amount of about 25%, about 50%, and about 5%, respectively, by weight of the base formulation;
c) phosphoric acid in an amount of about 0.005% by weight of the base formulation; and
d) hydroxypropyl cellulose in an amount of from 0.5% to about 2% by weight of the base formulation.

In some embodiments of any one of formulations (AB1ca), (AB1aa), and (AB1pa), the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

In some embodiments, the formulation (AC) includes:
a) the compound of formula (I);
b) a base formulation including:
   b2) the unsaturated fatty alcohol in an amount of from about 1% to about 10% by weight of the base formulation; and
   b3) C₁₋₃ alkyl-(OCH₂CH₂)₁₋₅—OH, the C₂₋₆ alkylene glycol, the di-(C₂₋₆ alkylene) glycol, and the polyethylene glycol;
c) an acid; and
d) the gelling agent,
   wherein:
the formulation is free of DMSO;
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
C₁₋₃ alkyl-(OCH₂CH₂)₁₋₅—OH, the C₂₋₆ alkylene glycol, the di-(C₂₋₆ alkylene) glycol, the polyethylene glycol, the unsaturated fatty alcohol, and the gelling agent are as described herein.

In some embodiments of formulation (AC), the unsaturated fatty alcohol is oleyl alcohol. In some embodiments, oleyl alcohol is present in the formulation in an amount of from about 1% to about 10%, from about 2% to about 10%, from about 2% to about 8%, from about 2% to about 6%, from about 3% to about 6%, from about 4% to about 6%, from about 2% to about 5%, from about 2% to about 4%, or from about 3% to about 5% by weight of the base formulation. In some embodiments, oleyl alcohol is present in an amount of from about 4% to about 6%, from about 2% to about 4%, or from about 3% to about 5% by weight of the base formulation. In some embodiments, oleyl alcohol is present in an amount of from about 4% to about 6% by weight of the base formulation. In some embodiments, oleyl alcohol is present in an amount of about 2%, about 3%, about 4%, about 4.5%, or about 5% by weight of the base formulation. In some embodiments, oleyl alcohol is present in an amount of about 3% by weight of the base formulation. In some embodiments, oleyl alcohol is present in an amount of about 4% by weight of the base formulation. In some embodiments, oleyl alcohol is present in an amount of about 4.5% by weight of the base formulation. In some embodiments, oleyl alcohol is present in an amount of about 5% by weight of the base formulation.

In some embodiments of formulation (AC), C₁₋₃ alkyl-(OCH₂CH₂)₁₋₅—OH and the polyethylene glycol are present in the formulation in a total amount of from about 40% to about 50% by weight of the base formulation. In some embodiments, C₁₋₃ alkyl-(OCH₂CH₂)₁₋₅—OH, and the polyethylene glycol are present in a total amount of about 45% by weight of the base formulation. In some embodiments, 2-(2-ethoxyethoxy)ethanol and PEG400 are present in a total amount of from about 40% to about 50% by weight of the base formulation. In some embodiments, 2-(2-ethoxyethoxy)ethanol and PEG400 are present in a total amount of about 45% by weight of the base formulation.

In some embodiments of formulation (AC), C₁₋₃ alkyl-(OCH₂CH₂)₁₋₅—OH is 2-(2-ethoxyethoxy)ethanol present in the formulation in an amount of from about 20% to about 40%, from about 20% to about 35%, or from about 20% to about 30% by weight of the base formulation. In some embodiments, 2-(2-ethoxyethoxy)ethanol is present in an amount of from about 20% to about 30% by weight of the base formulation. In some embodiments, 2-(2-ethoxyethoxy)ethanol is present in an amount of about 23% by weight of the base formulation.

In some embodiments of formulation (AC), the C₂₋₆ alkylene glycol is propylene glycol present in the formulation in an amount of from about 30% to about 70%, from about 30% to about 60%, from about 40% to about 60%, or from about 40% to about 50% by weight of the base formulation. In some embodiments, propylene glycol is present in an amount of from about 40% to about 60% or from about 40% to about 50% by weight of the base formulation. In some embodiments, propylene glycol is present in an amount of from about 40% to about 60% by weight of the base formulation. In some embodiments, propylene glycol is present in an amount of from about 40% to about 50% by weight of the base formulation. In some embodiments, propylene glycol is present in an amount of about 45.5% by weight of the base formulation.

In some embodiments of formulation (AC), the di-(C₂₋₆ alkylene) glycol is dipropylene glycol present in the formulation in an amount of from about 1% to about 10%, from about 2% to about 8%, from about 3% to about 7%, or from about 4% to about 6% by weight of the base formulation. In some embodiments, dipropylene glycol is present in an amount of from about 3% to about 7% or from about 4% to about 6% by weight of the base formulation. In some embodiments, dipropylene glycol is present in an amount of from about 4% to about 6% by weight of the base formulation. In some embodiments, dipropylene glycol is present in an amount of about 4.5% by weight of the base formulation.

In some embodiments of formulation (AC), the polyethylene glycol is PEG400 present in the formulation in an amount of from about 15% to about 30% by weight of the base formulation. In some embodiments, PEG400 is present in an amount of from about 20% to about 25% by weight of the base formulation. In some embodiments, PEG400 is present in an amount of about 22.5% by weight of the base formulation.

In some embodiments of formulation (AC), the gelling agent is hydroxypropyl cellulose. In some embodiments, hydroxypropyl cellulose is present in an amount of from about 0.5% to about 2% by weight of the base formulation, while the formulation has a viscosity of from about 5,000 cP to about 20,000 cP. In some embodiments, hydroxypropyl cellulose is present in an amount of from about 0.5% to about 2% by weight of the base formulation, while the formulation has a viscosity of from about 10,000 cP to about 20,000 cP. In some embodiments, the hydroxypropyl cellulose having an average molecular weight of from about 700,000 Da to about 1,150,000 Da is present in an amount of from 0.5% to about 2% by weight of the base formulation. In some embodiments, the hydroxypropyl cellulose having an average molecular weight of from about 700,000 Da to about 1,150,000 Da is present in an amount of from 0.5% to about 2% by weight of the base formulation, while the formulation has a viscosity of from about 5,000 cP to about 20,000 cP. In some embodiments, the hydroxypropyl cellulose having an average molecular weight of from about 700,000 Da to about 1,150,000 Da is present in an amount of from 0.5% to about 2% by weight of the base formulation, while the formulation has a viscosity of from about 10,000 cP to about 20,000 cP.

In some embodiments of formulation (AC), the acid is citric acid and citric acid is present in the formulation in an amount of from about 0.01% to about 0.1% by weight of the base formulation. In some embodiments, citric acid is present in an amount of about 0.05% by weight of the base formulation. In some embodiments, citric acid is present in an amount of about 0.1% by weight of the base formulation.

In some embodiments of formulation (AC), the acid is acetic acid and acetic acid is present in the formulation in an amount of from about 0.01% to about 0.5% by weight of the base formulation. In some embodiments, acetic acid is present in an amount of about 0.05% by weight of the base formulation.

In some embodiments of formulation (AC), the acid is phosphoric acid and phosphoric acid is present in the formulation in an amount of from about 0.001% to about 0.01% by weight of the base formulation. In some embodiments, phosphoric acid is present in an amount of about 0.005% by weight of the base formulation.

In some embodiments, the formulation (AC1ea) includes:
a) the compound of formula (I);
b) a base formulation including:
   b2) oleyl alcohol in an amount of about 4.5% by weight of the base formulation; and
   b3) 2-(2-ethoxyethoxy)ethanol, propylene glycol, dipropylene glycol, and PEG400, which are present in an amount of about 23%, about 45.5%, about 4.5%, and about 22.5%, respectively, by weight of the base formulation;
c) citric acid in an amount of about 0.05% by weight of the base formulation; and
d) hydroxypropyl cellulose in an amount of from about 0.5% to about 2% by weight of the base formulation,
   wherein the formulation is free of DMSO.

In some embodiments, the formulation (AC1aa) includes:
a) the compound of formula (I);
b) a base formulation including:
   b2) oleyl alcohol in an amount of about 4.5% by weight of the base formulation; and
   b3) 2-(2-ethoxyethoxy)ethanol, propylene glycol, dipropylene glycol, and PEG400, which are present in an amount of about 23%, about 45.5%, about 4.5%, and about 22.5%, respectively, by weight of the base formulation;
c) acetic acid in an amount of about 0.05% by weight of the base formulation; and
d) hydroxypropyl cellulose in an amount of from about 0.5% to about 2% by weight of the base formulation,
   wherein the formulation is free of DMSO.

In some embodiments, the formulation (AC1pa) includes:
a) the compound of formula (I);
b) a base formulation including:
   b2) oleyl alcohol in an amount of about 4.5% by weight of the base formulation; and
   b3) 2-(2-ethoxyethoxy)ethanol, propylene glycol, dipropylene glycol, and PEG400, which are present in an amount of about 23%, about 45.5%, about 4.5%, and about 22.5%, respectively, by weight of the base formulation;
c) phosphoric acid in an amount of about 0.005% by weight of the base formulation; and
d) hydroxypropyl cellulose in an amount of from about 0.5% to about 2% by weight of the base formulation,
   wherein the formulation is free of DMSO.

In some embodiments of any one of formulations (AC1ea), (AC1aa), and (AC1pa), the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

In some embodiments, the formulation (AAca-1) includes:
a) from about 0.5% to about 5% by weight of the compound of formula (I), on a salt-free and anhydrous basis;
b) a base formulation including:
   b1) from about 3% to about 7% by weight of DMSO;
   b2) from about 2% to about 6% by weight of oleyl alcohol;
   b3-1) from about 20% to about 30% by weight of 2-(2-ethoxyethoxy)ethanol;
   b3-2) from about 40% to about 60% by weight of propylene glycol;
   b3-3) from about 4% to about 6% by weight of dipropylene glycol; and
   b3-4) from about 5% to about 15% by weight of PEG400;
c) from about 0.01% to about 0.1% by weight of citric acid; and
d) from about 0.5% to about 2% by weight of hydoxypropyl cellulose,
   wherein the total weight of from b1) to b3-4) is 100%; and the term “by weight” refers to “by weight of the base formulation”.

In some embodiments, the formulation (AA1ca-1) includes:
a) from about 0.5% to about 5% by weight of the compound of formula (I), on a salt-free and anhydrous basis;
b) a base formulation including:
   b1) about 5% by weight of DMSO;
   b2) about 5% by weight of oleyl alcohol;
   b3-1) about 25% by weight of 2-(2-ethoxyethoxy)ethanol;
   b3-2) about 50% by weight of propylene glycol;
   b3-3) about 5% by weight of dipropylene glycol; and
   b3-4) about 10% by weight of PEG400;
c) about 0.05% by weight of citric acid; and
d) about 2% by weight of hydoxypropyl cellulose,
   wherein the total weight of from b1) to b3-4) is 100%; and the term “by weight” refers to “by weight of the base formulation”.

In some embodiments, the formulation (AA2ca-1) includes:
a) from about 0.5% to about 5% by weight of the compound of formula (I), on a salt-free and anhydrous basis;
b) a base formulation including:
   b1) about 5% by weight of DMSO;
   b2) about 3% by weight of oleyl alcohol;
   b3-1) about 25% by weight of 2-(2-ethoxyethoxy)ethanol;
   b3-2) about 52% by weight of propylene glycol;
   b3-3) about 5% by weight of dipropylene glycol; and
   b3-4) about 10% by weight of PEG400;
c) about 0.05% by weight of citric acid; and
d) about 2% by weight of hydoxypropyl cellulose,
   wherein the total weight of from b1) to b3-4) is 100%; and the term “by weight” refers to “by weight of the base formulation”.

In some embodiments, the formulation (AAaa-1) includes:
a) from about 0.5% to about 5% by weight of the compound of formula (I), on a salt-free and anhydrous basis;
b) a base formulation including:
   b1) from about 3% to about 7% by weight of DMSO;
   b2) from about 2% to about 6% by weight of oleyl alcohol;
   b3-1) from about 20% to about 30% by weight of 2-(2-ethoxyethoxy)ethanol;
   b3-2) from about 40% to about 60% by weight of propylene glycol;
   b3-3) from about 4% to about 6% by weight of dipropylene glycol; and
   b3-4) from about 5% to about 15% by weight of PEG400;
c) from about 0.01% to about 0.5% by weight of acetic acid; and
d) from about 0.5% to about 2% by weight of hydoxypropyl cellulose,
   wherein the total weight of from b1) to b3-4) is 100%; and the term “by weight” refers to “by weight of the base formulation”.

In some embodiments, the formulation (AA1aa-1) includes:
a) from about 0.5% to about 5% by weight of the compound of formula (I), on a salt-free and anhydrous basis;
b) a base formulation including:
   b1) about 5% by weight of DMSO;
   b2) about 5% by weight of oleyl alcohol;
   b3-1) about 25% by weight of 2-(2-ethoxyethoxy)ethanol;
   b3-2) about 50% by weight of propylene glycol;
   b3-3) about 5% by weight of dipropylene glycol; and
   b3-4) about 10% by weight of PEG400;
c) about 0.05% by weight of acetic acid; and
d) about 2% by weight of hydoxypropyl cellulose,
   wherein the total weight of from b1) to b3-4) is 100%; and the term “by weight” refers to “by weight of the base formulation”.

In some embodiments, the formulation (AA2aa-1) includes:
a) from about 0.5% to about 5% by weight of the compound of formula (I), on a salt-free and anhydrous basis;
b) a base formulation including:
   b1) about 5% by weight of DMSO;
   b2) about 3% by weight of oleyl alcohol;
   b3-1) about 25% by weight of 2-(2-ethoxyethoxy)ethanol;
   b3-2) about 52% by weight of propylene glycol;
   b3-3) about 5% by weight of dipropylene glycol; and
   b3-4) about 10% by weight of PEG400;
c) about 0.05% by weight of acetic acid; and
d) about 2% by weight of hydoxypropyl cellulose,
   wherein the total weight of from b1) to b3-4) is 100%; and the term “by weight” refers to “by weight of the base formulation”.

In some embodiments, the formulation (AApa-1) includes:
a) from about 0.5% to about 5% by weight of the compound of formula (I), on a salt-free and anhydrous basis;
b) a base formulation including:
   b1) from about 3% to about 7% by weight of DMSO;
   b2) from about 2% to about 6% by weight of oleyl alcohol;
   b3-1) from about 20% to about 30% by weight of 2-(2-ethoxyethoxy)ethanol;
   b3-2) from about 40% to about 60% by weight of propylene glycol;
   b3-3) from about 4% to about 6% by weight of dipropylene glycol; and
   b3-4) from about 5% to about 15% by weight of PEG400;
c) from about 0.001% to about 0.01% by weight of phosphoric acid; and
d) from about 0.5% to about 2% by weight of hydoxypropyl cellulose,
   wherein the total weight of from b1) to b3-4) is 100%; and the term “by weight” refers to “by weight of the base formulation”.

In some embodiments, the formulation (AA1pa-1) includes:
a) from about 0.5% to about 5% by weight of the compound of formula (I), on a salt-free and anhydrous basis;
b) a base formulation including:
   b1) about 5% by weight of DMSO;
   b2) about 5% by weight of oleyl alcohol;
   b3-1) about 25% by weight of 2-(2-ethoxyethoxy)ethanol;
   b3-2) about 50% by weight of propylene glycol;
   b3-3) about 5% by weight of dipropylene glycol; and
   b3-4) about 10% by weight of PEG400;
c) about 0.005% by weight of phosphoric acid; and
d) about 2% by weight of hydoxypropyl cellulose,
   wherein the total weight of from b1) to b3-4) is 100%; and the term “by weight” refers to “by weight of the base formulation”.

In some embodiments, the formulation (AA2pa-1) includes:
a) from about 0.5% to about 5% by weight of the compound of formula (I), on a salt-free and anhydrous basis;
b) a base formulation including:
   b1) about 5% by weight of DMSO;
   b2) about 3% by weight of oleyl alcohol;
   b3-1) about 25% by weight of 2-(2-ethoxyethoxy)ethanol;
   b3-2) about 52% by weight of propylene glycol;
   b3-3) about 5% by weight of dipropylene glycol; and
   b3-4) about 10% by weight of PEG400;
c) about 0.005% by weight of phosphoric acid; and
d) about 2% by weight of hydoxypropyl cellulose,
   wherein the total weight of from b1) to b3-4) is 100%; and the term “by weight” refers to “by weight of the base formulation”.

In some embodiments, the formulation (ABca-1) includes:
a) from about 0.5% to about 5% by weight of the compound of formula (I), on a salt-free and anhydrous basis;
b) a base formulation including:
   b1) from about 5% to about 17% by weight of DMSO;
   b2) from about 2% to about 6% by weight of oleyl alcohol;
   b3-1) from about 20% to about 40% by weight of 2-(2-ethoxyethoxy)ethanol;
   b3-2) from about 40% to about 60% by weight of propylene glycol; and
   b3-3) from about 4% to about 6% by weight of dipropylene glycol;
c) from about 0.01% to about 0.1% by weight of citric acid; and
d) from about 0.5% to about 2% by weight of hydoxypropyl cellulose,
   wherein the total weight of from b1) to b3-3) is 100%; and the term “by weight” refers to “by weight of the base formulation”.

In some embodiments, the formulation (AB1ca-1) includes:
a) from about 1% to about 5% by weight of the compound of formula (I), on a salt-free and anhydrous basis;
b) a base formulation including:
   b1) about 15% by weight of DMSO;
   b2) about 5% by weight of oleyl alcohol;
   b3-1) about 25% by weight of 2-(2-ethoxyethoxy)ethanol;
   b3-2) about 50% by weight of propylene glycol; and
   b3-3) about 5% by weight of dipropylene glycol;
c) about 0.05% by weight of citric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1ca-2) includes:
a) from about 1% to about 5% by weight of the compound of formula (I), on a salt-free and anhydrous basis;
b) a base formulation including:
   b1) about 15% by weight of DMSO;
   b2) about 3% by weight of oleyl alcohol;
   b3-1) about 27% by weight of 2-(2-ethoxyethoxy)ethanol;
   b3-2) about 50% by weight of propylene glycol; and
   b3-3) about 5% by weight of dipropylene glycol;
c) about 0.05% by weight of citric acid; and
d) about 2% by weight of hydoxypropyl cellulose,
wherein the total weight of from b1) to b3-3) is 100%; and the term “by weight” refers to “by weight of the base formulation”.

In some embodiments, the formulation (AB1ca-3) includes:
a) from about 1% to about 5% by weight of the compound of formula (I), on a salt-free and anhydrous basis;
b) a base formulation including:
   b1) about 10% by weight of DMSO;
   b2) about 4% by weight of oleyl alcohol;
   b3-1) about 31% by weight of 2-(2-ethoxyethoxy)ethanol;
   b3-2) about 50% by weight of propylene glycol; and
   b3-3) about 5% by weight of dipropylene glycol;
c) about 0.05% by weight of citric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1ca-4) includes:
a) from about 1% to about 5% by weight of the compound of formula (I), on a salt-free and anhydrous basis;
b) a base formulation including:
   b1) about 10% by weight of DMSO;
   b2) about 3% by weight of oleyl alcohol;
   b3-1) about 32% by weight of 2-(2-ethoxyethoxy)ethanol;
   b3-2) about 50% by weight of propylene glycol; and
   b3-3) about 5% by weight of dipropylene glycol;
c) about 0.05% by weight of citric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1ca-5) includes:
a) from about 1% to about 5% by weight of the compound of formula (I), on a salt-free and anhydrous basis;
b) a base formulation including:
   b1) about 7.5% by weight of DMSO;
   b2) about 4% by weight of oleyl alcohol;
   b3-1) about 33.5% by weight of 2-(2-ethoxyethoxy)ethanol;
   b3-2) about 50% by weight of propylene glycol; and
   b3-3) about 5% by weight of dipropylene glycol;
c) about 0.05% by weight of citric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1ca-6) includes:
a) from about 1% to about 5% by weight of the compound of formula (I), on a salt-free and anhydrous basis;
b) a base formulation including:
   b1) about 7.5% by weight of DMSO;
   b2) about 3% by weight of oleyl alcohol;
   b3-1) about 34.5% by weight of 2-(2-ethoxyethoxy)ethanol;
   b3-2) about 50% by weight of propylene glycol; and
   b3-3) about 5% by weight of dipropylene glycol;
c) about 0.05% by weight of citric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (ABaa-1) includes:
a) from about 0.5% to about 5% by weight of the compound of formula (I), on a salt-free and anhydrous basis;
b) a base formulation including:
   b1) from about 5% to about 17% by weight of DMSO;
   b2) from about 2% to about 6% by weight of oleyl alcohol;
   b3-1) from about 20% to about 40% by weight of 2-(2-ethoxyethoxy)ethanol;
   b3-2) from about 40% to about 60% by weight of propylene glycol; and
   b3-3) from about 4% to about 6% by weight of dipropylene glycol;
c) from about 0.01% to about 0.5% by weight of acetic acid; and
d) from about 0.5% to about 2% by weight of hydoxypropyl cellulose,
   wherein the total weight of from b1) to b3-3) is 100%; and the term “by weight” refers to “by weight of the base formulation”.

In some embodiments, the formulation (AB1aa-1) includes:
a) from about 1% to about 5% by weight of the compound of formula (I), on a salt-free and anhydrous basis;
b) a base formulation including:
   b1) about 15% by weight of DMSO;
   b2) about 5% by weight of oleyl alcohol;
   b3-1) about 25% by weight of 2-(2-ethoxyethoxy)ethanol;
   b3-2) about 50% by weight of propylene glycol; and
   b3-3) about 5% by weight of dipropylene glycol;
c) about 0.05% by weight of acetic acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1aa-2) includes:
a) from about 1% to about 5% by weight of the compound of formula (I), on a salt-free and anhydrous basis;
b) a base formulation including:
   b1) about 15% by weight of DMSO;
   b2) about 3% by weight of oleyl alcohol;
   b3-1) about 27% by weight of 2-(2-ethoxyethoxy)ethanol;
   b3-2) about 50% by weight of propylene glycol; and
   b3-3) about 5% by weight of dipropylene glycol;
c) about 0.05% by weight of acetic acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1aa-3) includes:
a) from about 1% to about 5% by weight of the compound of formula (I), on a salt-free and anhydrous basis;
b) a base formulation including:
   b1) about 10% by weight of DMSO;
   b2) about 4% by weight of oleyl alcohol;
   b3-1) about 31% by weight of 2-(2-ethoxyethoxy)ethanol;
   b3-2) about 50% by weight of propylene glycol; and
   b3-3) about 5% by weight of dipropylene glycol;
c) about 0.05% by weight of acetic acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1aa-4) includes:
a) from about 1% to about 5% by weight of the compound of formula (I), on a salt-free and anhydrous basis;
b) a base formulation including:
   b1) about 10% by weight of DMSO;
   b2) about 3% by weight of oleyl alcohol;
   b3-1) about 32% by weight of 2-(2-ethoxyethoxy)ethanol;
   b3-2) about 50% by weight of propylene glycol; and
   b3-3) about 5% by weight of dipropylene glycol;
c) about 0.05% by weight of acetic acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1aa-5) includes:
a) from about 1% to about 5% by weight of the compound of formula (I), on a salt-free and anhydrous basis;
b) a base formulation including:
   b1) about 7.5% by weight of DMSO;
   b2) about 4% by weight of oleyl alcohol;
   b3-1) about 33.5% by weight of 2-(2-ethoxyethoxy)ethanol;
   b3-2) about 50% by weight of propylene glycol; and
   b3-3) about 5% by weight of dipropylene glycol;
c) about 0.05% by weight of acetic acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1aa-6) includes:
a) from about 1% to about 5% by weight of the compound of formula (I), on a salt-free and anhydrous basis;
b) a base formulation including:
   b1) about 7.5% by weight of DMSO;
   b2) about 3% by weight of oleyl alcohol;
   b3-1) about 34.5% by weight of 2-(2-ethoxyethoxy)ethanol;
   b3-2) about 50% by weight of propylene glycol; and
   b3-3) about 5% by weight of dipropylene glycol;
c) about 0.05% by weight of acetic acid; and
d) about 2% by weight of hydoxypropyl cellulose,
   wherein the total weight of from b1) to b3-3) is 100%; and the term “by weight” refers to “by weight of the base formulation”.

In some embodiments, the formulation (ABpa-1) includes:
a) from about 0.5% to about 5% by weight of the compound of formula (I), on a salt-free and anhydrous basis;
b) a base formulation including:
   b1) from about 5% to about 17% by weight of DMSO;
   b2) from about 2% to about 6% by weight of oleyl alcohol;
   b3-1) from about 20% to about 40% by weight of 2-(2-ethoxyethoxy)ethanol;
   b3-2) from about 40% to about 60% by weight of propylene glycol;
   b3-3) from about 4% to about 6% by weight of dipropylene glycol; and
c) from about 0.001% to about 0.01% by weight of phosphoric acid; and
d) from about 1% to about 3% by weight of hydoxypropyl cellulose,
   wherein the total weight of from b1) to b3-3) is 100%; and the term “by weight” refers to “by weight of the base formulation”.

In some embodiments, the formulation (AB1pa-1) includes:
a) from about 1% to about 5% by weight of the compound of formula (I), on a salt-free and anhydrous basis;
b) a base formulation including:
   b1) about 15% by weight of DMSO;
   b2) about 5% by weight of oleyl alcohol;
   b3-1) about 25% by weight of 2-(2-ethoxyethoxy)ethanol;
   b3-2) about 50% by weight of propylene glycol; and
   b3-3) about 5% by weight of dipropylene glycol;
c) about 0.005% by weight of phosphoric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1pa-2) includes:
a) from about 1% to about 5% by weight of the compound of formula (I), on a salt-free and anhydrous basis;
b) a base formulation including:
   b1) about 15% by weight of DMSO;
   b2) about 3% by weight of oleyl alcohol;
   b3-1) about 27% by weight of 2-(2-ethoxyethoxy)ethanol;
   b3-2) about 50% by weight of propylene glycol; and
   b3-3) about 5% by weight of dipropylene glycol;
c) about 0.005% by weight of phosphoric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1pa-3) includes:
a) from about 1% to about 5% by weight of the compound of formula (I), on a salt-free and anhydrous basis;
b) a base formulation including:
   b1) about 10% by weight of DMSO;
   b2) about 4% by weight of oleyl alcohol;
   b3-1) about 31% by weight of 2-(2-ethoxyethoxy)ethanol;
   b3-2) about 50% by weight of propylene glycol;
   b3-3) about 5% by weight of dipropylene glycol;
c) about 0.005% by weight of phosphoric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1pa-4) includes:
a) from about 1% to about 5% by weight of the compound of formula (I), on a salt-free and anhydrous basis;
b) a base formulation including:
   b1) about 10% by weight of DMSO;
   b2) about 3% by weight of oleyl alcohol;
   b3-1) about 32% by weight of 2-(2-ethoxyethoxy)ethanol;
   b3-2) about 50% by weight of propylene glycol; and
   b3-3) about 5% by weight of dipropylene glycol;
c) about 0.005% by weight of phosphoric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1pa-5) includes:
a) from about 1% to about 5% by weight of the compound of formula (I), on a salt-free and anhydrous basis;
b) a base formulation including:
   b1) about 7.5% by weight of DMSO;
   b2) about 4% by weight of oleyl alcohol;
   b3-1) about 33.5% by weight of 2-(2-ethoxyethoxy)ethanol;
   b3-2) about 50% by weight of propylene glycol; and
   b3-3) about 5% by weight of dipropylene glycol;
c) about 0.005% by weight of phosphoric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (AB1pa-6) includes:
a) from about 1% to about 5% by weight of the compound of formula (I), on a salt-free and anhydrous basis;
b) a base formulation including:
   b1) about 7.5% by weight of DMSO;
   b2) about 3% by weight of oleyl alcohol;
   b3-1) about 34.5% by weight of 2-(2-ethoxyethoxy)ethanol;
   b3-2) about 50% by weight of propylene glycol; and
   b3-3) about 5% by weight of dipropylene glycol;
c) about 0.005% by weight of phosphoric acid; and
d) about 2% by weight of hydoxypropyl cellulose,
   wherein the total weight of from b1) to b3-3) is 100%; and the term “by weight” refers to “by weight of the base formulation”.

In some embodiments, the formulation (ACca-1) includes:
a) from about 0.5% to about 5% by weight of the compound of formula (I), on a salt-free and anhydrous basis;
b) a base formulation including:
   b2) from about 2% to about 6% by weight of oleyl alcohol;
   b3-1) from about 20% to about 30% by weight of 2-(2-ethoxyethoxy)ethanol;
   b3-2) from about 40% to about 60% by weight of propylene glycol;
   b3-3) from about 4% to about 6% by weight of dipropylene glycol; and
   b3-4) from about 15% to about 30% by weight of PEG400;
c) from about 0.01% to about 0.1% by weight of citric acid; and
d) from about 0.5% to about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (AC1ca-1) includes:
a) from about 0.5% to about 5% by weight of the compound of formula (I), on a salt-free and anhydrous basis;
b) a base formulation including:
   b2) about 4.5% by weight of oleyl alcohol;
   b3-1) about 23% by weight of 2-(2-ethoxyethoxy)ethanol;
   b3-2) about 45.5% by weight of propylene glycol;
   b3-3) about 4.5% by weight of dipropylene glycol; and
   b3-4) about 22.5% by weight of PEG400;
c) about 0.05% by weight of citric acid; and
d) about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (AC1ca-2) includes:
a) from about 0.5% to about 5% by weight of the compound of formula (I), on a salt-free and anhydrous basis;
b) a base formulation including:
   b2) about 3% by weight of oleyl alcohol;
   b3-1) about 23% by weight of 2-(2-ethoxyethoxy)ethanol;
   b3-2) about 47% by weight of propylene glycol;
   b3-3) about 4.5% by weight of dipropylene glycol; and
   b3-4) about 22.5% by weight of PEG400;
c) about 0.05% by weight of citric acid; and
d) about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (ACaa-1) includes:
a) from about 0.5% to about 5% by weight of the compound of formula (I), on a salt-free and anhydrous basis;
b) a base formulation including:
   b2) from about 2% to about 6% by weight of oleyl alcohol;
   b3-1) from about 20% to about 30% by weight of 2-(2-ethoxyethoxy)ethanol;
   b3-2) from about 40% to about 60% by weight of propylene glycol;
   b3-3) from about 4% to about 6% by weight of dipropylene glycol; and
   b3-4) from about 15% to about 30% by weight of PEG400;
c) from about 0.01% to about 0.5% by weight of acetic acid; and
d) from about 0.5% to about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (AC1aa-1) includes:
a) from about 0.5% to about 5% by weight of the compound of formula (I), on a salt-free and anhydrous basis;
b) a base formulation including:
   b2) about 4.5% by weight of oleyl alcohol;
   b3-1) about 23% by weight of 2-(2-ethoxyethoxy)ethanol;
   b3-2) about 45.5% by weight of propylene glycol;
   b3-3) about 4.5% by weight of dipropylene glycol; and
   b3-4) about 22.5% by weight of PEG400;
c) about 0.05% by weight of acetic acid; and
d) about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (AC1aa-2) includes:
a) from about 0.5% to about 5% by weight of the compound of formula (I), on a salt-free and anhydrous basis;
b) a base formulation including:
   b2) about 3% by weight of oleyl alcohol;
   b3-1) about 23% by weight of 2-(2-ethoxyethoxy)ethanol;
   b3-2) about 47% by weight of propylene glycol;
   b3-3) about 4.5% by weight of dipropylene glycol; and
   b3-4) about 22.5% by weight of PEG400;
c) about 0.05% by weight of acetic acid; and
d) about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (ACpa-1) includes:
a) from about 0.5% to about 5% by weight of the compound of formula (I), on a salt-free and anhydrous basis;
b) a base formulation including:
   b2) from about 2% to about 6% by weight of oleyl alcohol;
   b3-1) from about 20% to about 30% by weight of 2-(2-ethoxyethoxy)ethanol;
   b3-2) from about 40% to about 60% by weight of propylene glycol;
   b3-3) from about 4% to about 6% by weight of dipropylene glycol; and
   b3-4) from about 15% to about 30% by weight of PEG400;
c) from about 0.001% to about 0.01% by weight of phosphoric acid; and
d) from about 0.5% to about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (AC1pa-1) includes:
a) from about 0.5% to about 5% by weight of the compound of formula (I), on a salt-free and anhydrous basis;
b) a base formulation including:
   b2) about 4.5% by weight of oleyl alcohol;
   b3-1) about 23% by weight of 2-(2-ethoxyethoxy)ethanol;
   b3-2) about 45.5% by weight of propylene glycol;
   b3-3) about 4.5% by weight of dipropylene glycol; and
   b3-4) about 22.5% by weight of PEG400;
c) about 0.005% by weight of phosphoric acid; and
d) about 2% by weight of hydoxypropyl cellulose,
   wherein the total weight of from b2) to b3-4) is 100%; the formulation is free of DMSO; and the term “by weight” refers to “by weight of the base formulation”.

In some embodiments, the formulation (AC1ca-2) includes:
a) from about 0.5% to about 5% by weight of the compound of formula (I), on a salt-free and anhydrous basis;
b) a base formulation including:
   b2) about 3% by weight of oleyl alcohol;
   b3-1) about 23% by weight of 2-(2-ethoxyethoxy)ethanol;
   b3-2) about 47% by weight of propylene glycol;
   b3-3) about 4.5% by weight of dipropylene glycol; and
   b3-4) about 22.5% by weight of PEG400;
c) about 0.005% by weight of phosphoric acid; and
d) about 2% by weight of hydoxypropyl cellulose,
   wherein the total weight of from b2) to b3-4) is 100%; the formulation is free of DMSO; and the term “by weight” refers to “by weight of the base formulation”.

With reference to any one of formulations (AA1ca), (AA2ca), (AA1aa), (AA2aa), (AA1pa), (AA2pa), (AB1ca), (AB1aa), (AB1pa), (AC1ca), (AC1aa), and (AC1pa) as described herein, in some embodiments, the hydroxypropyl cellulose has an average molecular weight of about 850,000 Da (e.g., Klucel MF). In some embodiments, the hydroxypropyl cellulose has an average molecular weight of about 1,150,000 Da (e.g., Klucel HF). In some embodiments, the hydroxypropyl cellulose has an average molecular weight of about 700,000 Da (e.g., Nisso M). In some embodiments, the hydroxypropyl cellulose has an average molecular weight of about 1,000,000 Da (e.g., Nisso H).

With reference to any one of formulations (AA), (AA1ca), (AA2ca), (AA1aa), (AA2aa), (AA1pa), (AA2pa), (AB), (AB1ca), (AB1aa), (AB1pa), (AC), (AC1ca), (AC1aa), and (AC1pa) as described herein, the compounds of formula (I) are described according to Section IIIA-7. In some embodiments, the compounds of formula (I) is represented by formula (II-1a). In some embodiments, the compound of formula (II-1a) is present in a degree to saturation of from about 75% to about 100%. In some embodiments, the compound of formula (II-1a) is present in a degree to saturation of from about 90% to about 100%. In some embodiments, the compound of formula (II-1a) is present in a degree to saturation of about 100% (i.e., at a saturated concentration).

### IIIB. Formulations Including DMSO

In a second aspect, the present disclosure provides a formulation including:
a) a compound having formula (I):

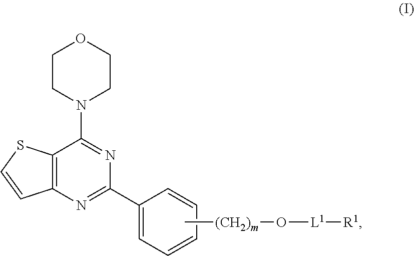

a hydrate, a solvate, a pharmaceutically acceptable salt, or a combination thereof;
wherein:
   subscript m is an integer from 0 to 2;
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl; and
b) a base formulation including:
   b1) DMSO in an amount of from about 1% to about 20% by weight of the base formulation;
   b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
   b3) one or more solvents including a C₂₋₆ alkylene glycol, a di-(C₂₋₆ alkylene) glycol, or a combination thereof,
      wherein:
the ratio of DMSO to the combined one or more excipients is at least about 1:1 by weight; and
hydrolysis of the compound having formula (I) to a corresponding compound having formula (IV):

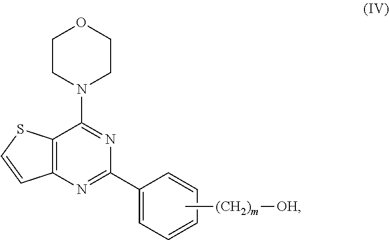

is less than about 10% over a period of about 10 days at a temperature of 80° C. (±2° C.) or over a period of about 6 months at a temperature of 40° C. (±2° C.) and a relative humidity of 75% (±5%), wherein subscript m is an integer from 0 to 2.

Without further being bound to a particular theory, it is believed that DMSO may act as an anti-oxidant and is capable of reducing the hydroperoxides, as described in Section IIIA, for example produced by oleyl alcohol, oleic acid, an ester, and an ether thereof.

The formulation can be prepared by methods, for example mixing components of a) and b) (e.g., components of a), b1), b2), and b3)). In order to control the hydrolysis of the compound having formula (I) to the corresponding compound having formula (IV) to be less than about 10% over a period of about 10 days at a temperature of 80° C. (±2° C.) or over a period of about 6 months at a temperature of 40° C. (±2° C.) and a relative humidity of 75% (±5%), in some embodiments, the formulation is prepared by sonicating a mixture of b1) to b2) followed by mixing with remaining components (e.g., a) and b3)). In some embodiments, the formulation is prepared by sonicating a mixture of a) and b) (e.g., components of a), b1), b2), and b3)).

The formulation can be a topical formulation suitable for a topical application. In some embodiments, the formulation is a topical formulation. In some embodiments, the formulation is suitable for a topical application.

### IIIB-1. Excipients

The one or more excipients are as defined and described according to Section IIIA-1.

In some embodiments, the one or more excipients consist essentially of oleyl alcohol. In some embodiments, one or more excipients are oleyl alcohol; and oleyl alcohol is present in an amount of from about 1% to about 10% by weight of the base formulation. In some embodiments, oleyl alcohol is present in an amount of from about 1% to about 10%, from about 2% to about 7%, from about 3% to about 7%, from about 3% to about 6%, or from about 3% to about 5% by weight of the base formulation. In some embodiments, oleyl alcohol is present in an amount of from about 3% to about 6%, or from about 3% to about 5% by weight of the base formulation. In some embodiments, oleyl alcohol is present in an amount of from about 3% to about 6% by weight of the base formulation. In some embodiments, oleyl alcohol is present in an amount of from about 3% to about 5% by weight of the base formulation. In some embodiments, oleyl alcohol is present in an amount of about 5% by weight of the base formulation.

### IIIB-2. DMSO

The content of DMSO is described in the embodiments according to Section IIIA-2.

In some embodiments, the ratio of DMSO to the combined one or more excipients is from about 1:1 to about 10:1 by weight. In some embodiments, the ratio of DMSO to the combined one or more excipients is from about 1:1 to about 5:1 by weight. In some embodiments, the ratio of DMSO to the combined one or more excipients is from about 1:1 to about 3:1 by weight. In some embodiments, the ratio of DMSO to the combined one or more excipients is from about 1:1 by weight. In some embodiments, the ratio of DMSO to the combined one or more excipients is about 3:1 by weight. In some embodiments, the one or more excipients consist essentially of oleyl alcohol.

In some embodiments, the one or more excipients are oleyl alcohol; and the ratio of DMSO to oleyl alcohol is at least about 1:1 by weight. In some embodiments, the ratio of DMSO to oleyl alcohol is from about 1:1 to about 10:1 by weight. In some embodiments, the ratio of DMSO to oleyl alcohol is from about 1:1 to about 5:1 by weight. In some embodiments, the ratio of DMSO to oleyl alcohol is from about 1:1 to about 3:1 by weight. In some embodiments, the ratio of DMSO to oleyl alcohol is about 1:1 by weight. In some embodiments, the ratio of DMSO to oleyl alcohol is about 3:1 by weight.

### IIIB-3. Solvents

The one or more solvents are defined and described according to Section IIIA-4.

Embodiments related to a C₂₋₆ alkylene glycol, a di-(C₂₋₆ alkylene) glycol, C₁₋₃ alkyl-(OCH₂CH₂)₁₋₅—OH, and/or a polyethylene glycol are described according to Section IIIA-4.

In some embodiments, the one or more solvents include 2-(2-ethoxyethoxy)ethanol, propylene glycol, and dipropylene glycol. In some embodiments, the one or more solvents include 2-(2-ethoxyethoxy)ethanol, propylene glycol, dipropylene glycol, and PEG400.

Embodiments related to 2-(2-ethoxyethoxy)ethanol, propylene glycol, and/or dipropylene glycol are described according to Section IIIA-4.

With reference to PEG400, in some embodiments, PEG400 is present in the formulation in an amount of from about 5% to about 50%, from about 5% to about 40%, from about 5% to about 30%, from about 5% to about 20%, or from about 5% to about 15% by weight of the base formulation. In some embodiments, PEG400 is present in an amount of from about 5% to about 30%, from about 5% to about 20%, or from about 5% to about 15% by weight of the base formulation. In some embodiments, PEG400 is present in an amount of from about 5% to about 20% or from about 5% to about 15% by weight of the base formulation. In some embodiments, PEG400 is present in an amount of from about 5% to about 15% by weight of the base formulation. In some embodiments, PEG400 is present in an amount of about 10% by weight of the base formulation.

### IIIB-5. Viscosity

In some embodiments, the formulation has a viscosity, as defined and described according to Section IIIA-5 and Section IIIA-6.

### IIIB-5. Gelling Agent

In some embodiments, the formulation further includes a gelling agent. The gelling agent is defined and described according to Section IIIA-6.

In some embodiments, the gelling agent is hydroxypropyl cellulose. In some embodiments, hydroxypropyl cellulose is present in the formulation in an amount of from about 0.5% to about 2% by weight of the base formulation, while the formulation has a viscosity of from about 5,000 cP to about 20,000 cP. In some embodiments, hydroxypropyl cellulose is present in an amount of from about 0.5% to about 2% by weight of the base formulation, while the formulation has a viscosity of from about 5,000 cP to about 15,000 cP. In some embodiments, hydroxypropyl cellulose is present in an amount of from about 0.5% to about 2% by weight of the base formulation, while the formulation has a viscosity of from about 10,000 cP to about 20,000 cP.

In some embodiments, the hydroxypropyl cellulose having an average molecular weight of from about 700,000 Da to about 1,150,000 Da is present in an amount of from about 0.5% to about 2% by weight of the base formulation. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 20,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 15,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 10,000 cP to about 20,000 cP.

In some embodiments, the hydroxypropyl cellulose having an average molecular weight of from about 700,000 Da to about 1,150,000 Da is present in an amount of about 1% by weight of the base formulation. In some embodiments, the hydroxypropyl cellulose having an average molecular weight of from about 700,000 Da to about 1,150,000 Da is present in an amount of about 2% by weight of the base formulation.

### IIIB-6. Compounds of Formula (I)

The compound of formula (I), a hydrate, a solvate, a pharmaceutically acceptable salt, or a combination thereof, is further described according to Section V. COMPOUNDS.

Embodiments related to compounds of formula (I) are described according to Section IIIA-7.

In some embodiments, the compound of formula (I) in any one of the formulations is represented by formula (IIa-1a):

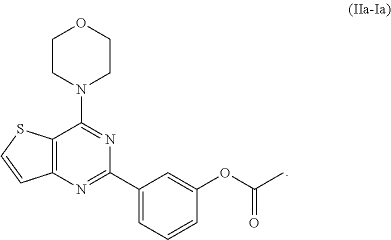 [SMILES: CC(=O)Oc1cccc(-c2nc(N3CCOCC3)c3sccc3n2)c1]

In some embodiments, the compound of formula (IIa-1) used in preparing the formulation is in a salt-free form.

In some embodiments of any one of formulations, the compound of formula (IIa-1) is present in a degree to saturation of from about 50% to about 100%. In some embodiments, the compound of formula (IIa-1) is present in a degree to saturation of from about 75% to about 100%. In some embodiments, the compound of formula (IIa-1) is present in a degree to saturation of from about 80% to about 100%, from about 90% to about 100%, or about 100%. In some embodiments, the compound of formula (IIa-1) is present in a degree to saturation of from about 90% to about 100%. In some embodiments, the compound of formula (IIa-1) is present at a saturated concentration in the formulation (i.e., a degree to saturation of about 100%).

### IIIB-7. Selected Embodiments

In some embodiments, the formulation (BA) includes:
a) the compound of formula (I);
b) a base formulation including:
   b1) DMSO in an amount of from about 1% to about 10% by weight of the base formulation;
   b2) the unsaturated fatty alcohol in an amount of from about 1% to about 10% by weight of the base formulation; and
   b3) C₁₋₃ alkyl-(OCH₂CH₂)₁₋₅—OH, the C₂₋₆ alkylene glycol, the di-(C₂₋₆ alkylene) glycol, and the polyethylene glycol; and
c) the gelling agent,
   wherein C₁₋₃ alkyl-(OCH₂CH₂)₁₋₅—OH, the C₂₋₆ alkylene glycol, the di-(C₂₋₆ alkylene) glycol, the polyethylene glycol, the unsaturated fatty alcohol, and the gelling agent are as described herein.

In some embodiments of formulation (BA), the unsaturated fatty alcohol is oleyl alcohol. In some embodiments, oleyl alcohol is present in the formulation in an amount of from about 1% to about 10%, from about 2% to about 10%, from about 2% to about 8%, from about 2% to about 6%, from about 3% to about 6%, from about 4% to about 6%, from about 2% to about 5%, from about 2% to about 4%, or from about 3% to about 5% by weight of the base formulation. In some embodiments, oleyl alcohol is present in an amount of from about 4% to about 6%, from about 2% to about 4%, or from about 3% to about 5% by weight of the base formulation. In some embodiments, oleyl alcohol is present in an amount of from about 4% to about 6% by weight of the base formulation. In some embodiments, oleyl alcohol is present in an amount of about 2%, about 3%, about 4%, or about 5% by weight of the base formulation. In some embodiments, oleyl alcohol is present in an amount of about 3% by weight of the base formulation. In some embodiments, oleyl alcohol is present in an amount of about 4% by weight of the base formulation. In some embodiments, oleyl alcohol is present in an amount of about 5% by weight of the base formulation.

In some embodiments of formulation (BA), DMSO, C₁₋₃ alkyl-(OCH₂CH₂)₁₋₅—OH, and the polyethylene glycol are present in the formulation in a total amount of from about 35% to about 45% by weight of the base formulation. In some embodiments, DMSO, C₁₋₃ alkyl-(OCH₂CH₂)₁₋₅—OH, and the polyethylene glycol are present in a total amount of about 40% by weight of the base formulation. In some embodiments, DMSO, 2-(2-ethoxyethoxy)ethanol, and PEG400 are present in a total amount of from about 35% to about 45% by weight of the base formulation. In some embodiments, DMSO, 2-(2-ethoxyethoxy)ethanol and PEG400 are present in a total amount of about 40% by weight of the base formulation.

In some embodiments of formulation (BA), DMSO is present in the formulation in an amount of from about 3% to about 7% by weight of the base formulation. In some embodiments, DMSO is present in an amount of about 5% by weight of the base formulation.

In some embodiments of formulation (BA), C₁₋₃ alkyl-(OCH₂CH₂)₁₋₅—OH is 2-(2-ethoxyethoxy)ethanol and 2-(2-ethoxyethoxy)ethanol is present in the formulation in an amount of from about 20% to about 40%, from about 20% to about 35%, or from about 20% to about 30% by weight of the base formulation. In some embodiments, 2-(2-ethoxyethoxy)ethanol is present in an amount of from about 20% to about 30% by weight of the base formulation. In some embodiments, 2-(2-ethoxyethoxy)ethanol is present in an amount of about 25% by weight of the base formulation.

In some embodiments of formulation (BA), the C₂₋₆ alkylene glycol is propylene glycol and propylene glycol is present in the formulation in an amount of from about 30% to about 70%, from about 30% to about 60%, from about 40% to about 60%, or from about 45% to about 55% by weight of the base formulation. In some embodiments, propylene glycol is present in an amount of from about 40% to about 60% or from about 45% to about 55% by weight of the base formulation. In some embodiments, propylene glycol is present in an amount of from about 40% to about 60% by weight of the base formulation. In some embodiments, propylene glycol is present in an amount of from about 45% to about 55% by weight of the base formulation. In some embodiments, propylene glycol is present in an amount of about 50% by weight of the base formulation. In some embodiments, propylene glycol is present in an amount of about 52% by weight of the base formulation.

In some embodiments of formulation (BA), the di-(C₂₋₆ alkylene) glycol is dipropylene glycol and dipropylene glycol is present in the formulation in an amount of from about 1% to about 10%, from about 2% to about 8%, from about 3% to about 7%, or from about 4% to about 6% by weight of the base formulation. In some embodiments, dipropylene glycol is present in an amount of from about 3% to about 7% or from about 4% to about 6% by weight of the base formulation. In some embodiments, dipropylene glycol is present in an amount of from about 4% to about 6% by weight of the base formulation. In some embodiments, dipropylene glycol is present in an amount of about 5% by weight of the base formulation.

In some embodiments of formulation (BA), the polyethylene glycol is PEG400 is present in the formulation in an amount of from about 5% to about 15% by weight of the base formulation. In some embodiments, PEG400 is present in an amount of from about 5% to about 15% by weight of the base formulation. In some embodiments, PEG400 is present in an amount of about 10% by weight of the base formulation.

In some embodiments of formulation (BA), the gelling agent is hydroxypropyl cellulose. In some embodiments, hydroxypropyl cellulose is present in an amount of from about 0.5% to about 2% by weight of the base formulation, while the formulation has a viscosity of from about 5,000 cP to about 20,000 cP. In some embodiments, hydroxypropyl cellulose is present in an amount of from about 0.5% to about 2% by weight of the base formulation, while the formulation has a viscosity of from about 10,000 cP to about 20,000 cP. In some embodiments, the hydroxypropyl cellulose having an average molecular weight of from about 700,000 Da to about 1,150,000 Da is present in an amount of from 0.5% to about 2% by weight of the base formulation. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 20,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 10,000 cP to about 20,000 cP.

In some embodiments, the formulation (BA1) includes:
a) the compound of formula (I);
b) a base formulation including:
   b1) DMSO in an amount of about 5% by weight of the base formulation;
   b2) oleyl alcohol in an amount of about 5% by weight of the base formulation; and
   b3) 2-(2-ethoxyethoxy)ethanol, propylene glycol, dipropylene glycol, and PEG400, which are present in an amount of about 25%, about 50%, about 5%, and about 10%, respectively, by weight of the base formulation; and
c) hydroxypropyl cellulose in an amount of from about 0.5% to about 2% by weight of the base formulation.

In some embodiments, the formulation (BA2) includes:
a) the compound of formula (I);
b) a base formulation including:
   b1) DMSO in an amount of about 5% by weight of the base formulation;
   b2) oleyl alcohol in an amount of about 3% by weight of the base formulation; and
   b3) 2-(2-ethoxyethoxy)ethanol, propylene glycol, dipropylene glycol, and PEG400, which are present in an amount of about 25%, about 52%, about 5%, and about 10%, respectively, by weight of the base formulation; and
c) hydroxypropyl cellulose in an amount of about 0.5% to about 2% by weight of the base formulation.

In some embodiments of formulation (BA1) or (BA2), the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

In some embodiments, the formulation (BB) includes:
a) the compound of formula (I);
b) a base formulation including:
   b1) DMSO in an amount of from about 5% to about 17% by weight of the base formulation;
   b2) the unsaturated fatty alcohol in an amount of from about 1% to about 10% by weight of the base formulation; and
   b3) C₁₋₃ alkyl-(OCH₂CH₂)₁₋₅—OH, the C₂₋₆ alkylene glycol, and the di-(C₂₋₆ alkylene) glycol; and
c) the gelling agent,
wherein C₁₋₃ alkyl-(OCH₂CH₂)₁₋₅—OH, the C₂₋₆ alkylene glycol, the di-(C₂₋₆ alkylene) glycol, the unsaturated fatty alcohol, and the gelling agent are as described herein.

In some embodiments of formulation (BB), the unsaturated fatty alcohol is oleyl alcohol. In some embodiments, oleyl alcohol is present in the formulation in an amount of from about 1% to about 10%, from about 2% to about 10%, from about 2% to about 8%, from about 2% to about 6%, from about 3% to about 6%, from about 4% to about 6%, from about 2% to about 5%, from about 2% to about 4%, or from about 3% to about 5% by weight of the base formulation. In some embodiments, oleyl alcohol is present in an amount of from about 4% to about 6%, from about 2% to about 4%, or from about 3% to about 5% by weight of the base formulation. In some embodiments, oleyl alcohol is present in an amount of from about 4% to about 6% by weight of the base formulation. In some embodiments, oleyl alcohol is present in an amount of about 2%, about 3%, about 4%, or about 5% by weight of the base formulation. In some embodiments, oleyl alcohol is present in an amount of about 3% by weight of the base formulation. In some embodiments, oleyl alcohol is present in an amount of about 4% by weight of the base formulation. In some embodiments, oleyl alcohol is present in an amount of about 5% by weight of the base formulation.

In some embodiments of formulation (BB), DMSO and C₁₋₃ alkyl-(OCH₂CH₂)₁₋₅—OH are present in the formulation in a total amount of from about 35% to about 45% by weight of the base formulation. In some embodiments, DMSO and C₁₋₃ alkyl-(OCH₂CH₂)₁₋₅—OH are present in a total amount of about 40% by weight of the base formulation. In some embodiments, DMSO and 2-(2-ethoxyethoxy)ethanol are present in a total amount of from about 35% to about 45% by weight of the base formulation. In some embodiments, DMSO and 2-(2-ethoxyethoxy)ethanol are present in a total amount of about 40% by weight of the base formulation.

In some embodiments of formulation (BB), DMSO is present in the formulation in an amount of from about 5% to about 15% by weight of the base formulation. In some embodiments, DMSO is present in an amount of about 7.5%, about 10%, or about 15% by weight of the base formulation. In some embodiments, DMSO is present in an amount of about 15% by weight of the base formulation.

In some embodiments of formulation (BB), C₁₋₃ alkyl-(OCH₂CH₂)₁₋₅—OH is 2-(2-ethoxyethoxy)ethanol and 2-(2-ethoxyethoxy)ethanol is present in the formulation in an amount of from about 20% to about 40%, from about 20% to about 35%, from about 20% to about 30%, from about 25% to about 35%, from about 25% to about 30%, from about 30% to about 35%, or from about 30% to about 40% by weight of the base formulation. In some embodiments, 2-(2-ethoxyethoxy)ethanol is present in an amount of about 34.5%, about 33.5%, about 32%, about 31%, about 27%, or about 25% by weight of the base formulation. In some embodiments, 2-(2-ethoxyethoxy)ethanol is present in an amount of about 34.5% by weight of the base formulation. In some embodiments, 2-(2-ethoxyethoxy)ethanol is present in an amount of about 33.5% by weight of the base formulation. In some embodiments, 2-(2-ethoxyethoxy)ethanol is present in an amount of about 32% by weight of the base formulation. In some embodiments, 2-(2-ethoxyethoxy)ethanol is present in an amount of about 31% by weight of the base formulation. In some embodiments, 2-(2-ethoxyethoxy)ethanol is present in an amount of about 27% by weight of the base formulation. In some embodiments, 2-(2-ethoxyethoxy)ethanol is present in an amount of about 25% by weight of the base formulation.

In some embodiments of formulation (BB), the C₂₋₆ alkylene glycol is propylene glycol and propylene glycol is present in the formulation in an amount of from about 30% to about 70%, from about 30% to about 60%, from about 40% to about 60%, or from about 45% to about 55% by weight of the base formulation. In some embodiments, propylene glycol is present in an amount of from about 40% to about 60% or from about 45% to about 55% by weight of the base formulation. In some embodiments, propylene glycol is present in an amount of from about 40% to about 60% by weight of the base formulation. In some embodiments, propylene glycol is present in an amount of from about 45% to about 55% by weight of the base formulation. In some embodiments, propylene glycol is present in an amount of about 50% by weight of the base formulation.

In some embodiments of formulation (BB), the di-(C₂₋₆ alkylene) glycol is dipropylene glycol and dipropylene glycol is present in the formulation in an amount of from about 1% to about 10%, from about 2% to about 8%, from about 3% to about 7%, or from about 4% to about 6% by weight of the base formulation. In some embodiments, dipropylene glycol is present in an amount of from about 3% to about 7% or from about 4% to about 6% by weight of the base formulation. In some embodiments, dipropylene glycol is present in an amount of from about 4% to about 6% by weight of the base formulation. In some embodiments, dipropylene glycol is present in an amount of about 5% by weight of the base formulation.

In some embodiments of formulation (BB), the gelling agent is hydroxypropyl cellulose. In some embodiments, hydroxypropyl cellulose is present in an amount of from about 0.5% to about 2% by weight of the base formulation, while the formulation has a viscosity of from about 5,000 cP to about 20,000 cP. In some embodiments, hydroxypropyl cellulose is present in an amount of from about 0.5% to about 2% by weight of the base formulation, while the formulation has a viscosity of from about 10,000 cP to about 20,000 cP. In some embodiments, the hydroxypropyl cellulose having an average molecular weight of from about 700,000 Da to about 1,150,000 Da is present in an amount of from 0.5% to about 2% by weight of the base formulation. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 5,000 cP to about 20,000 cP. In some embodiments, the hydroxypropyl cellulose having an average molecular weight of from about 700,000 Da to about 1,150,000 Da is present in an amount of from about 0.5% to about 2% by weight of the base formulation, while the formulation has a viscosity of from about 10,000 cP to about 20,000 cP.

In some embodiments, the formulation (BB1) includes:
a) the compound of formula (I);
b) a base formulation including:
   b1) DMSO in an amount of about 15% by weight of the base formulation;
   b2) oleyl alcohol in an amount of about 5% by weight of the base formulation; and
   b3) 2-(2-ethoxyethoxy)ethanol, propylene glycol, and dipropylene glycol, which are present in an amount of about 25%, about 50%, and about 5%, respectively, by weight of the base formulation; and
c) hydroxypropyl cellulose in an amount of from about 0.5 to about 2% by weight of the base formulation.

In some embodiments of formulation (BB1), the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

In some embodiments, the formulation (BA-1) includes:
a) from about 0.5% to about 5% by weight of the compound of formula (I), on a salt-free and anhydrous basis;
b) a base formulation including:
   b1) from about 3% to about 7% by weight of DMSO;
   b2) from about 2% to about 6% by weight of oleyl alcohol;
   b3-1) from about 20% to about 30% by weight of 2-(2-ethoxyethoxy)ethanol;
   b3-2) from about 40% to about 60% by weight of propylene glycol;
   b3-3) from about 4% to about 6% by weight of dipropylene glycol; and
   b3-4) from about 5% to about 15% by weight of PEG400; and
c) from about 0.5% to about 2% by weight of hydoxypropyl cellulose,
   wherein the total weight of from b1) to b3-4) is 100%, and the term “by weight” refers to “by weight of the base formulation”.

In some embodiments, the formulation (BA1-1) includes:
a) from about 0.5% to about 5% by weight of the compound of formula (I), on a salt-free and anhydrous basis;
b) a base formulation including:
   b1) about 5% by weight of DMSO;
   b2) about 5% by weight of oleyl alcohol;
   b3-1) about 25% by weight of 2-(2-ethoxyethoxy)ethanol;
   b3-2) about 50% by weight of propylene glycol;
   b3-3) about 5% by weight of dipropylene glycol; and
   b3-4) about 10% by weight of PEG400; and
c) about 2% by weight of hydoxypropyl cellulose,
   wherein the total weight of from b1) to b3-4) is 100%, and the term “by weight” refers to “by weight of the base formulation”.

In some embodiments, the formulation (BA2-1) includes:
a) from about 0.5% to about 5% by weight of the compound of formula (I), on a salt-free and anhydrous basis;
b) a base formulation including:
   b1) about 5% by weight of DMSO;
   b2) about 3% by weight of oleyl alcohol;
   b3-1) about 25% by weight of 2-(2-ethoxyethoxy)ethanol;
   b3-2) about 52% by weight of propylene glycol;
   b3-3) about 5% by weight of dipropylene glycol; and
   b3-4) about 10% by weight of PEG400; and
c) about 2% by weight of hydoxypropyl cellulose,
   wherein the total weight of from b1) to b3-4) is 100%, and the term “by weight” refers to “by weight of the base formulation”.

In some embodiments, the formulation (BB-1) includes:
a) from about 0.5% to about 5% by weight of the compound of formula (I), on a salt-free and anhydrous basis;
b) a base formulation including:
   b1) from about 5% to about 17% DMSO by weight;
   b2) from about 2% to about 6% by weight of oleyl alcohol;
   b3-1) from about 20% to about 40% by weight of 2-(2-ethoxyethoxy)ethanol;
   b3-2) from about 40% to about 60% by weight of propylene glycol; and
   b3-3) from about 4% to about 6% by weight of dipropylene glycol; and
c) from about 1% to about 3% by weight of hydoxypropyl cellulose,
   wherein the total weight of from b1) to b3-3) is 100%, and the term “by weight” refers to “by weight of the base formulation”.

In some embodiments, the formulation (BB1-1) includes:
a) from about 1% to about 5% by weight of the compound of formula (I), on a salt-free and anhydrous basis;
b) a base formulation including:
   b1) about 15% by weight of DMSO;
   b2) about 5% by weight of oleyl alcohol;
   b3-1) about 25% by weight of 2-(2-ethoxyethoxy)ethanol;
   b3-2) about 50% by weight of propylene glycol; and
   b3-3) about 5% by weight of dipropylene glycol; and
c) about 2% by weight of hydoxypropyl cellulose,
   wherein the total weight of from b1) to b3-3) is 100%, and the term “by weight” refers to “by weight of the base formulation”.

In some embodiments, the formulation (BB1-2) includes:
a) from about 1% to about 5% by weight of the compound of formula (I), on a salt-free and anhydrous basis;
b) a base formulation including:
   b1) about 15% by weight of DMSO;
   b2) about 3% by weight of oleyl alcohol;
   b3-1) about 27% by weight of 2-(2-ethoxyethoxy)ethanol;
   b3-2) about 50% by weight of propylene glycol; and
   b3-3) about 5% by weight of dipropylene glycol; and
c) about 2% by weight of hydoxypropyl cellulose,
   wherein the total weight of from b1) to b3-3) is 100%, and the term “by weight” refers to “by weight of the base formulation”.

In some embodiments, the formulation (BB1-3) includes:
a) from about 1% to about 5% by weight of the compound of formula (I), on a salt-free and anhydrous basis;
b) a base formulation including:
   b1) about 10% by weight of DMSO;
   b2) about 4% by weight of oleyl alcohol;
   b3-1) about 31% by weight of 2-(2-ethoxyethoxy)ethanol;
   b3-2) about 50% by weight of propylene glycol; and
   b3-3) about 5% by weight of dipropylene glycol; and
c) about 2% by weight of hydoxypropyl cellulose,
   wherein the total weight of from b1) to b3-3) is 100%, and the term “by weight” refers to “by weight of the base formulation”.

In some embodiments, the formulation (BB1-4) includes:
a) from about 1% to about 5% by weight of the compound of formula (I), on a salt-free and anhydrous basis;
b) a base formulation including:
   b1) about 10% by weight of DMSO;
   b2) about 3% by weight of oleyl alcohol;
   b3-1) about 32% by weight of 2-(2-ethoxyethoxy)ethanol;
   b3-2) about 50% by weight of propylene glycol; and
   b3-3) about 5% by weight of dipropylene glycol; and
c) about 2% by weight of hydoxypropyl cellulose,
   wherein the total weight of from b1) to b3-3) is 100%, and the term “by weight” refers to “by weight of the base formulation”.

In some embodiments, the formulation (BB1-5) includes:
a) from about 1% to about 5% by weight of the compound of formula (I), on a salt-free and anhydrous basis;
b) a base formulation including:
   b1) about 7.5% by weight of DMSO;
   b2) about 4% by weight of oleyl alcohol;
   b3-1) about 33.5% by weight of 2-(2-ethoxyethoxy)ethanol;
   b3-2) about 50% by weight of propylene glycol; and
   b3-3) about 5% by weight of dipropylene glycol; and
c) about 2% by weight of hydoxypropyl cellulose,
   wherein the total weight of from b1) to b3-3) is 100%, and the term “by weight” refers to “by weight of the base formulation”.

In some embodiments, the formulation (BB1-6) includes:
a) from about 1% to about 5% by weight of the compound of formula (I), on a salt-free and anhydrous basis;
b) a base formulation including:
   b1) about 7.5% by weight of DMSO;
   b2) about 3% by weight of oleyl alcohol;
   b3-1) about 34.5% by weight of 2-(2-ethoxyethoxy)ethanol;
   b3-2) about 50% by weight of propylene glycol; and
   b3-3) about 5% by weight of dipropylene glycol; and
c) about 2% by weight of hydoxypropyl cellulose,
   wherein the total weight of from b1) to b3-3) is 100%, and the term “by weight” refers to “by weight of the base formulation”.

With reference to any one of formulations (BA-1), (BA1-1), (BA2-1), (BB-1), and (BB1-1) to (BB1-6) as described herein, the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

With reference to any one of formulations (BA), (BA-1), (BA1), (BA1-1), (BA2), (BA2-1), (BB), (BB-1), (BB1), and (BB1-1) to (BB1-6) as described herein, the compounds of formula (I) are described according to Section IIIA-7. In some embodiments, the compound of formula (I) is represented by formula (II-1a). In some embodiments, the compound of formula (II-1a) is present in a degree to saturation of from about 75% to about 100%. In some embodiments, the compound of formula (II-1a) is present in a degree to saturation of from about 90% to about 100%. In some embodiments, the compound of formula (II-1a) is present in a degree to saturation of about 100% (i.e., at a saturated concentration).

### IIIC. Stability of Formulations

In some embodiments, the formulations as described herein have a visual appearance as clear, transparent, and/or monophasic. In some embodiments, the visual appearance of the formulation is maintained over a period of about 10 days at a temperature of 80° C. (±2° C.). In some embodiments, the visual appearance of the formulation is maintained over a period of about 6 months at a temperature of 40° C. (±2° C.) and a relative humidity of 75% (±5%).

When the gelling agent is present in the formulation as described herein, in some embodiments, the topical gel formulation has stable viscosity for a period of about 10 days at a temperature of 80° C. (±2° C.) or over a period of about 6 months at a temperature of 40° C. (±2° C.) and a relative humidity of 75% (±5%). In some embodiments, the viscosity of the topical gel formulation is maintained from about 5,000 cP to about 100,000 cP over a period of about 10 days at a temperature of 80° C. (±2° C.) or over a period of about 6 months at a temperature of 40° C. (±2° C.) and a relative humidity of 75% (±5%). In some embodiments, the viscosity of the topical gel formulation is maintained from about 5,000 cP to about 50,000 cP over a period of about 10 days at a temperature of 80° C. (±2° C.) or over a period of about 6 months at a temperature of 40° C. (±2° C.) and a relative humidity of 75% (±5%). In some embodiments, the viscosity of the topical gel formulation is maintained from about 5,000 cP to about 20,000 cP over a period of about 10 days at a temperature of 80° C. (±2° C.) or over a period of about 6 months at a temperature of 40° C. (±2° C.) and a relative humidity of 75% (±5%). In some embodiments, the viscosity of the topical gel formulation is maintained from about 5,000 cP to about 15,000 cP over a period of about 10 days at a temperature of 80° C. (±2° C.) or over a period of about 6 months at a temperature of 40° C. (±2° C.) and a relative humidity of 75% (±5%). In some embodiments, the viscosity of the topical gel formulation is maintained from about 10,000 cP to about 20,000 cP over a period of about 10 days at a temperature of 80° C. (±2° C.) or over a period of about 6 months at a temperature of 40° C. (±2° C.) and a relative humidity of 75% (±5%).

The purity of the compound of formula (I) in the formulation can be determined by an analytical method, for example a high performance liquid chromatography (HPLC) method. In some embodiments, the compound of formula (I) has a relative purity of 100% in the formulation at time zero (i.e., day 0).

In some embodiments, the formulations as described herein provide suitable physical stability of the compound of formula (I) for a period of about 10 days at a temperature of 80° C. (±2° C.) or over a period of about 6 months at a temperature of 40° C. (±2° C.) and a relative humidity of 75% (±5%). In some embodiments, the relative purity of the compound having formula (I) in the formulation decreases less than about 10% over a period of about 10 days at a temperature of 80° C. (±2° C.) or over a period of about 6 months at a temperature of 40° C. (±2° C.) and a relative humidity of 75% (±5%). In some embodiments, the relative purity of the compound having formula (I) in the formulation decreases less than about 5% over a period of about 10 days at a temperature of 80° C. (±2° C.) or over a period of about 6 months at a temperature of 40° C. (±2° C.) and a relative humidity of 75% (±5%). In some embodiments, the relative purity of the compound having formula (I) in the formulation decreases less than about 2% over a period of about 10 days at a temperature of 80° C. (±2° C.) or over a period of about 6 months at a temperature of 40° C. (±2° C.) and a relative humidity of 75% (±5%). In some embodiments, the relative purity of the compound having formula (I) in the formulation decreases less than about 1% over a period of about 10 days at a temperature of 80° C. (±2° C.) or over a period of about 6 months at a temperature of 40° C. (±2° C.) and a relative humidity of 75% (±5%).

It is believed that the compound of formula (I) can hydrolyze to a corresponding compound of formula (IV) under certain conditions, as shown below:

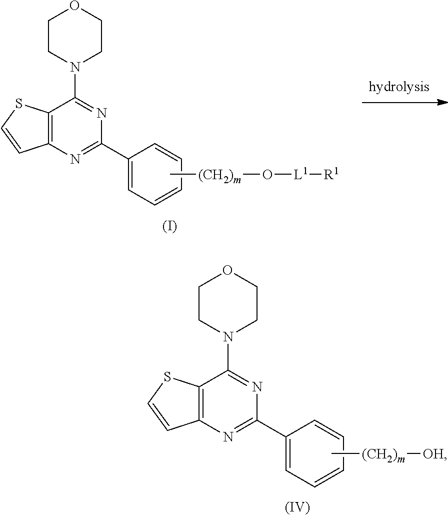

wherein subscript m, L¹, and R¹ are as defined and described herein.

In some embodiments, the hydrolysis of the compound having formula (I) in the formulation to a corresponding compound having formula (IV) is less than about 10% over a period of about 10 days at a temperature of 80° C. (±2° C.) or over a period of about 6 months at a temperature of 40° C. (±2° C.) and a relative humidity of 75% (±5%). In some embodiments, the hydrolysis of the compound having formula (I) in the formulation to a corresponding compound having formula (IV) is less than about 5% over a period of about 10 days at a temperature of 80° C. (±2° C.) or over a period of about 6 months at a temperature of 40° C. (±2° C.) and a relative humidity of 75% (±5%). In some embodiments, the hydrolysis of the compound having formula (I) in the formulation to a corresponding compound having formula (IV) is less than about 2% over a period of about 10 days at a temperature of 80° C. (±2° C.) or over a period of about 6 months at a temperature of 40° C. (±2° C.) and a relative humidity of 75% (±5%). In some embodiments, the hydrolysis of the compound having formula (I) in the formulation to a corresponding compound having formula (IV) is less than about 1% over a period of about 10 days at a temperature of 80° C. (±2° C.) or over a period of about 6 months at a temperature of 40° C. (±2° C.) and a relative humidity of 75% (±5%).

In some embodiments, the compound of formula (IIa-1a) can hydrolyze to a corresponding compound of formula (IVa) under certain conditions, as shown below:

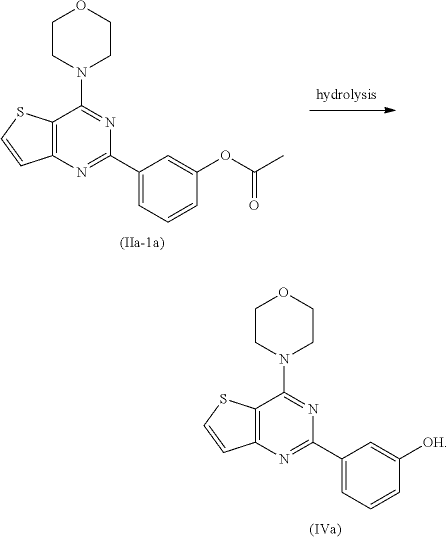

In some embodiments, the hydrolysis of formula (IIa-1a) in the formulation to formula (IVa) is less than about 10% over a period of about 10 days at a temperature of 80° C. (±2° C.) or over a period of about 6 months at a temperature of 40° C. (±2° C.) and a relative humidity of 75% (±5%). In some embodiments, the hydrolysis of formula (IIa-1a) in the formulation to formula (IVa) is less than about 5% over a period of about 10 days at a temperature of 80° C. (±2° C.) or over a period of about 6 months at a temperature of 40° C. (±2° C.) and a relative humidity of 75% (±5%). In some embodiments, the hydrolysis of formula (IIa-1a) in the formulation to formula (IVa) is less than about 2% over a period of about 10 days at a temperature of 80° C. (±2° C.) or over a period of about 6 months at a temperature of 40° C. (±2° C.) and a relative humidity of 75% (±5%). In some embodiments, the hydrolysis of formula (IIa-1a) in the formulation to formula (IVa) is less than about 1% over a period of about 10 days at a temperature of 80° C. (±2° C.) or over a period of about 6 months at a temperature of 40° C. (±2° C.) and a relative humidity of 75% (±5%).

In some embodiments, the compound of formula (IIIa-1a) can hydrolyze to a corresponding compound of formula (IVb) under certain conditions, as shown below:

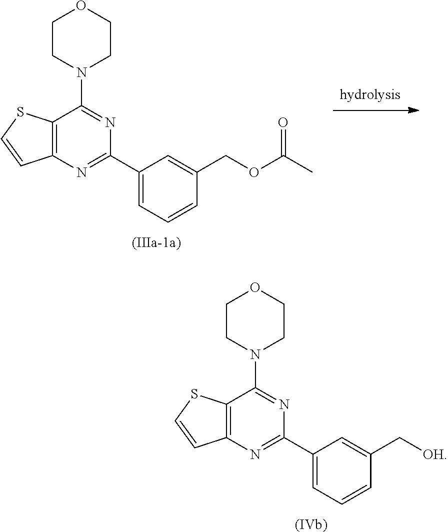

In some embodiments, the hydrolysis of formula (IIIa-1) in the formulation to formula (IVb) is less than about 10% over a period of about 10 days at a temperature of 80° C. (±2° C.) or over a period of about 6 months at a temperature of 40° C. (±2° C.) and a relative humidity of 75% (±5%). In some embodiments, the hydrolysis of formula (IIIa-1) in the formulation to formula (IVb) is less than about 5% over a period of about 10 days at a temperature of 80° C. (±2° C.) or over a period of about 6 months at a temperature of 40° C. (±2° C.) and a relative humidity of 75% (±5%). In some embodiments, the hydrolysis of formula (IIIa-1) in the formulation to formula (IVb) is less than about 2% over a period of about 10 days at a temperature of 80° C. (±2° C.) or over a period of about 6 months at a temperature of 40° C. (±2° C.) and a relative humidity of 75% (±5%). In some embodiments, the hydrolysis of formula (IIIa-1) in the formulation to formula (IVb) is less than about 1% over a period of about 10 days at a temperature of 80° C. (±2° C.) or over a period of about 6 months at a temperature of 40° C. (±2° C.) and a relative humidity of 75% (±5%).

### IIID. Permeation of Topical Formulations

In some embodiments, the topical formulations described herein provide enhanced skin permeation as compared to topical formulations including the compound of formula (IV) with the same composition (i.e., DMSO, the same one or more excipients, and one or more solvents). Skin permeability can be assessed by skin flux experiments in various animal skin models. In some embodiments, the skin flux of a topical formulation including a compound of formula (I) is at least about 2 fold greater than the skin flux of a formulation including the corresponding compound of formula (IV) where the respective formulations otherwise have the same composition, provided that the compound of formula (I) and the compound of formula (IV) have the same degree to saturation. In some embodiments, the skin flux of a topical formulation including a compound of formula (I) is about 2 to 5 fold greater than the skin flux of a formulation including the corresponding compound of formula (IV) where the respective formulations otherwise have the same composition, provided that the compound of formula (I) and the compound of formula (IV) have the same degree to saturation. In some embodiments, the skin flux of a topical formulation including the compound of formula (I) is about 2 fold greater than the skin flux of a formulation including the corresponding compound of formula (IV) where the respective formulations otherwise have the same composition, provided that the compound of formula (I) and the compound of formula (IV) have the same degree to saturation.

In some embodiments, the compound of formula (IIa-1a) has a cumulative skin flux of at least about 1 μg/cm²/hour, about 2 μg/cm²/hour, or about 3 μg/cm²/hour at about 24 hours, as measured by a Franz diffusion cell using a human cadaver skin. In some embodiments, the compound of formula (IIa-1a) has a cumulative skin flux of at least about 1 μg/cm²/hour at about 24 hours, as measured by a Franz diffusion cell using a human cadaver skin. In some embodiments, the compound of formula (IIa-1a) has a cumulative skin flux of at least about 2 μg/cm²/hour at about 24 hours, as measured by a Franz diffusion cell using a human cadaver skin. In some embodiments, the compound of formula (IIa-1a) has a cumulative skin flux of at least about 3 μg/cm²/hour at about 24 hours, as measured by a Franz diffusion cell using a human cadaver skin.

### IIIE. Forms of Topical Formulations

Topical formulations useful for delivering compounds of formula (I) to a subject (e.g., to the skin of a subject) include, but are not limited to, foams, sprays, aerosols, creams, lotions, ointments, gels, solutions, emulsions, and suspensions. See, e.g., REMINGTON: THE SCIENCE AND PRACTICE OF PHARMACY; (Alfonso R. Gennaro ed. 19th ed. 1995); and Introduction to Pharmaceutical Dosage Forms, 4th ed., Lea & Febiger, Philadelphia (1985). In some embodiments, the topical formulation used to deliver the compound of formula (I) is a foam, a lotion, a spray, an aerosol, an ointment, a cream, a gel, a paste, a patch, or an in-situ patch.

In some embodiments, the topical formulation used to deliver the compound of formula (I) is a lotion or a cream. Creams and lotions that can be used as topical formulations and their preparation are disclosed in REMINGTON: THE SCIENCE AND PRACTICE OF PHARMACY 282-291 (Alfonso R. Gennaro ed. 19th ed. 1995), the relevant portions of which are hereby incorporated herein by reference.

In some embodiments, the topical formulation used to deliver the compound of formula (I) is a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the disclosure are disclosed in REMINGTON: THE SCIENCE AND PRACTICE OF PHARMACY 1517-1518 (Alfonso R. Gennaro ed. 19th ed. 1995), which is hereby incorporated herein by reference. Other suitable gels for use with the disclosure are disclosed in U.S. Pat. No. 6,387,383 (issued May 14, 2002), U.S. Pat. No. 6,517,847 (issued Feb. 11, 2003), and U.S. Pat. No. 6,468,989 (issued Oct. 22, 2002), each of which is hereby incorporated herein by reference.

In some embodiments, the topical formulation used to deliver the compound of formula (I) is an ointment. Ointments are oleaginous semisolids that contain little if any water. In some instances, the ointment is hydrocarbon based, such as a wax, petrolatum, or gelled mineral oil. Suitable ointments for use according to the present disclosure include those disclosed in REMINGTON: THE SCIENCE AND PRACTICE OF PHARMACY 1585-1591 (Alfonso R. Gennaro ed. 19th ed. 1995).

In some embodiments, the topical administration may be achieved in the form of patches comprising the topical formulation as described herein. In some embodiments, the patch is in contact with the affected area on the skin. In some embodiments, the patch, when applied to a subject, is in contact with areas of the skin of the subject that are adjacent to the affected or target area.

In some embodiments, the topical administration may be achieved in the form of patches comprising the topical formulation as described herein. In some embodiments, the patch is in contact with the affected area on the skin. In some embodiments, the patch, when applied to a subject, is in contact with areas of the skin of the subject that are adjacent to the affected or target area.

In some embodiments, the topical administration may be achieved in the form of in-situ patches comprising the topical formulation as described herein. “In-situ patch” is formed when the topical formulation is sprayed or applied, one or more times and thus allowed to develop a thickness. In some embodiments, the in-situ patch is in contact with the affected or target area on the skin (e.g., the area where a vascular malformation is or has been present, as indicated via visual inspection, imaging, or the like). In some embodiments, the in-situ patch is in contact with areas of the skin of the subject that are adjacent to the affected or target.

### IIIF. Irritation of Topical Formulations

In some embodiments, the topical formulations provided herein provoke less skin irritation compared to other topical formulations. In some embodiments, a topical formulation provided herein including one or more excipients in an amount of less than about 10% by weight of the base formulation reduces skin irritation as compared to a topical formulation including the one or more excipients in an amount of greater than 10% by weight of the base formulation, wherein the one or more excipients are selected from the group consisting of an unsaturated fatty alcohol, an unsaturated fatty acid, an unsaturated fatty ester, an unsaturated fatty ether, and a combination thereof. In some embodiments, a topical formulation provided herein that includes DMSO and one or more excipients (e.g., oleyl alcohol) in a ratio of at least about 1:1 reduces skin irritation as compared to a topical formulation including DMSO and the one or more excipients in a ratio of less than about 1:1, wherein the one or more excipients are selected from the group consisting of an unsaturated fatty alcohol, an unsaturated fatty acid, an unsaturated fatty ester, an unsaturated fatty ether, a combination thereof. In some embodiments, the one or more excipients include oleyl alcohol. In some embodiments, the one or more excipients are oleyl alcohol.

Skin irritation may be assessed by a variety of mechanisms, including visual inspection, palpation, biopsy analysis, and patient self-reporting. Skin irritation assessment may comprise assessment of lesions, scaling, discoloration, dryness, erythema, edema, pain, itchiness, burning sensation, and related effects, including an area of a skin reaction. In some embodiments, a biopsy of an area to which a formulation provided herein (e.g., a formulation including a compound of formula (I)) is assessed (e.g., using a microscope) for hyperplasia and associated cellular infiltration. Assessment of skin irritation may be performed over the course of a therapeutic intervention for a patient involving the use of a formulation provided herein, such as prior to application of the formulation, immediately following application of the formulation, and at hourly, daily, weekly, or other intervals until the use of the formulation has ceased and/or the patient no longer reports skin irritation associated with the therapeutic intervention.

It is generally understood that the amount of highly reactive species such as peroxides (e.g., hydroperoxides) in a formulation correlates with the degree of irritation it may provoke upon administration to a patient (e.g., topical administration to a surface of a subject, such as to the skin of the subject). Peroxides (e.g., hydroperoxides) may be contaminants in a component such as an unsaturated fatty alcohol (e.g., oleyl alcohol), unsaturated fatty acid (e.g., oleic acid), unsaturated fatty ester, or unsaturated fatty ether included in a formulation and/or may form during later exposure of such a component to oxygen in air. Peroxides also contribute to undesirable hydrolysis of compounds of formula (I) under storage conditions. As described elsewhere herein, DMSO can act as an anti-oxidant and mitigate the effects of peroxides by reducing O—O bond in a polar process involving promotion with either acid or base.

Accordingly, exposure of unsaturated fatty acids and alcohols to DMSO, especially with heat and/or sonication, may reduce the amount of peroxides (e.g., hydroperoxides) included in a formulation. Details of such processes are described in the Examples and elsewhere herein. The ratio of DMSO to an unsaturated fatty acid, alcohol, ester, or ether in a formulation can also influence the formation of peroxides and thus the stability of a formulation under storage conditions, as well as the potential for the formulation to irritate the skin of a subject upon application.

Accordingly, in an aspect, the present disclosure provides a formulation (e.g., a topical formulation) including:
a) a compound having formula (I):

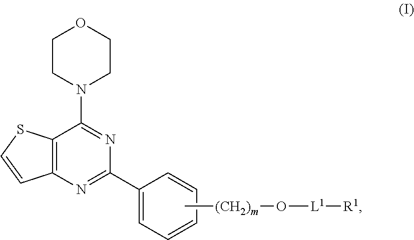

a hydrate, a solvate, a pharmaceutically acceptable salt, or a combination thereof;
wherein:
   subscript m is an integer from 0 to 2;
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) dimethyl sulfoxide (DMSO); and
c) one or more excipients selected from the group consisting of an unsaturated fatty alcohol, an unsaturated fatty acid, an unsaturated fatty ester, an unsaturated fatty ether, and a combination thereof;
   wherein DMSO is present in an amount of no more than about 15% by weight of the formulation, and wherein the ratio of DMSO to the combined one or more excipients is at least about 1:1 by weight.

In some embodiments, the ratio of DMSO to the combined one or more excipients is about 1:1. In some embodiments, the formulation provides reduced peroxide content after 10 days under accelerated storage conditions compared to to a formulation that includes a ratio of DMSO to the combined one or more excipients that is less than about 1:1. In some embodiments, the formulation provides enhanced stability compared to to a formulation that includes a ratio of DMSO to the combined one or more excipients that is less than about 1:1. In some embodiments, the formulation provides reduced skin irritation compared to a formulation that includes a ratio of DMSO to the combined one or more excipients that is less than about 1:1. In some embodiments, reduced skin irritation is indicated by reduced erythema, eschar, and/or edema formation and/or reduced area of skin reactions.

In some embodiments, the formulation includes DMSO in an amount of no more than about 10% by weight of the formulation. In some embodiments, the formulation includes DMSO in an amount of no more than about 5% by weight of the formulation.

In some embodiments, the one or more excipients comprise oleyl alcohol. In some embodiments, the one or more excipients consist essentially of oleyl alcohol. In some embodiments, the one or more excipients are oleyl alcohol.

In some embodiments, the formulation further comprises one or more solvents, such as one or more solvents selected from a C₂₋₆ alkylene glycol, a di-(C₂₋₆ alkylene) glycol, or a combination thereof. In some embodiments, the formulation further comprises a gelling agent such as HPC. In some embodiments, the formulation further comprises an acid such as citric acid, acetic acid, or phosphoric acid. In some embodiments, the amount of the acid in the formulation is no more than about about 1% by weight of the formulation.

In some embodiments, hydrolysis of the compound having formula (I) to a corresponding compound having formula (IV):

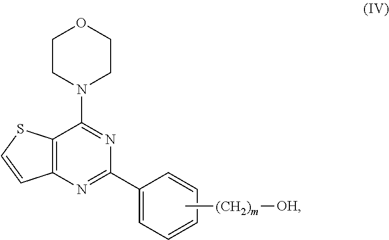

is less than about 10% over a period of about 10 days at a temperature of 80° C. (±2° C.) or over a period of about 6 months at a temperature of 40° C. (±2° C.) and a relative humidity of 75% (±5%), wherein subscript m is an integer from 0 to 2.

### IV. Processes

### IVA. Processes for Preparing Formulations Including an Acid

In a third aspect, the present disclosure provides a process for preparing a formulation according to the first aspect and embodiments as described herein. The process includes:
1) forming a first mixture including:
   a) a compound having formula (I):

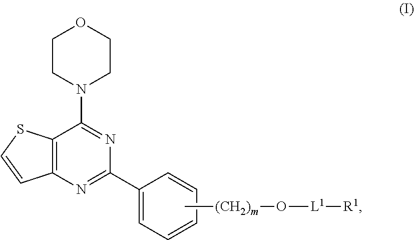

a hydrate, a solvate, a pharmaceutically acceptable salt, or a combination thereof;
wherein:
   subscript m is an integer from 0 to 2;
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) a base formulation including:
   b1) DMSO in an amount of from about 0% to about 20% by weight of the base formulation;
   b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
   b3) one or more solvents inluding a C₂₋₆ alkylene glycol, a di-(C₂₋₆ alkylene) glycol, or a combination thereof; and
c) an acid in an amount of no more than about 1% by weight of the base formulation, and
2) mixing the first mixture to form a uniform mixture.

The one or more solvents are defined and described according to Section IIIA-4. In some embodiments, the one or more solvents further include C₁₋₃ alkyl-(OCH₂CH₂)₁₋₅—OH, a polyethylene glycol, or a combination thereof. In some embodiments, the one or more solvents include C₁₋₃ alkyl-(OCH₂CH₂)₁₋₅—OH, a C₂₋₆ alkylene glycol, a di-(C₂₋₆ alkylene) glycol, a polyethylene glycol, or a combination thereof, provided that at least one of a C₂₋₆ alkylene glycol and a di-(C₂₋₆ alkylene) glycol is present. In some embodiments, the one or more solvents include a C₂₋₆ alkylene glycol; and further include C₁₋₃ alkyl-(OCH₂CH₂)₁₋₅—OH, a di-(C₂₋₆ alkylene) glycol, a polyethylene glycol, or a combination thereof.

Embodiments related to C₁₋₃ alkyl-(OCH₂CH₂)₁₋₅—OH, a C₂₋₆ alkylene glycol, a di-(C₂₋₆ alkylene) glycol, and/or a polyethylene glycol are described according to Section IIIA-4.

In some embodiments, the process includes:
1) forming a first mixture including:
   a) the compound of formula (I);
   b) a base formulation comprising:
      b1) DMSO in an amount of from about 0% to about 20% by weight of the base formulation;
      b2) an unsaturated fatty alcohol in an amount of no more than about 10% by weight of the base formulation; and
      b3) one or more solvents including C₁₋₃ alkyl-(OCH₂CH₂)₁₋₅—OH, a C₂₋₆ alkylene glycol, a di-(C₂₋₆ alkylene) glycol, a polyethylene glycol, or a combination thereof, provided that at least one of a C₂₋₆ alkylene glycol and a di-(C₂₋₆ alkylene) glycol is present; and
   c) an acid, and
2) mixing the first mixture to form a uniform mixture,
   wherein:
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
the unsaturated fatty alcohol, C₁₋₃ alkyl-(OCH₂CH₂)₁₋₅—OH, the C₂₋₆ alkylene glycol, the di-(C₂₋₆ alkylene) glycol, and the polyethylene glycol are as defined and described herein.

In some embodiments, the process includes:
1) forming a first mixture including:
   a) the compound of formula (I);
   b) a base formulation including:
      b1) DMSO in an amount of from about 0% to about 20% by weight of the base formulation;
      b2) an unsaturated fatty alcohol in an amount of no more than about 10% by weight of the base formulation; and
      b3) one or more solvents including a C₂₋₆ alkylene glycol and further include C₁₋₃ alkyl-(OCH₂CH₂)₁₋₅—OH, a di-(C₂₋₆ alkylene) glycol, a polyethylene glycol, or a combination thereof; and
   c) an acid, and
2) mixing the first mixture to form a uniform mixture,
   wherein:
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
the unsaturated fatty alcohol, C₁₋₃ alkyl-(OCH₂CH₂)₁₋₅—OH, the C₂₋₆ alkylene glycol, the di-(C₂₋₆ alkylene) glycol, and the polyethylene glycol are as defined and described herein.

In some embodiments, the process includes:
1) forming a first mixture including:
   a) the compound of formula (I);
   b) a base formulation including:
      b1) DMSO in an amount of from about 1% to about 20% by weight of the base formulation;
      b2) an unsaturated fatty alcohol in an amount of no more than about 10% by weight of the base formulation; and
      b3) one or more solvents including C₁₋₃ alkyl-(OCH₂CH₂)₁₋₅—OH, a C₂₋₆ alkylene glycol, a di-(C₂₋₆ alkylene) glycol, a polyethylene glycol, or a combination thereof, provided that at least one of a C₂₋₆ alkylene glycol and a di-(C₂₋₆ alkylene) glycol is present; and
   c) an acid, and
2) mixing the first mixture to form a uniform mixture,
   wherein:
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
the unsaturated fatty alcohol, C₁₋₃ alkyl-(OCH₂CH₂)₁₋₅—OH, the C₂₋₆ alkylene glycol, the di-(C₂₋₆ alkylene) glycol, and the polyethylene glycol are as defined and described herein.

In some embodiments, the process includes:
1) forming a first mixture including:
   a) the compound of formula (I);
   b) a base formulation including:
      b1) DMSO in an amount of from about 1% to about 20% by weight of the base formulation;
      b2) an unsaturated fatty alcohol in an amount of no more than about 10% by weight of the base formulation; and
      b3) one or more solvents including a C₂₋₆ alkylene glycol and further include C₁₋₃ alkyl-(OCH₂CH₂)₁₋₅—OH, a di-(C₂₋₆ alkylene) glycol, a polyethylene glycol, or a combination thereof; and
   c) an acid, and
2) mixing the first mixture to form a uniform mixture,
   wherein:
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
the unsaturated fatty alcohol, C₁₋₃ alkyl-(OCH₂CH₂)₁₋₅—OH, the C₂₋₆ alkylene glycol, the di-(C₂₋₆ alkylene) glycol, and the polyethylene glycol are as defined and described herein.

When DMSO is present, the process can further include pretreating any one or more of components of b1), b2) and c) by 1) heating b1), b2), and c), alone or in a combination, at an elevated temperature for a period of time; or 2) by forming a premixture of b1) and b2) or a premixture of b1), b2), and c), and mixing the premixture at room temperature for a period of time. When DMSO is absent, the process can further include pretreating any one or more of components of b2) and c) by 1) heating b2) and c), alone or in a combination, at an elevated temperature for a period of time; or 2) by forming a premixture of b2) and c) and mixing the premixture at room temperature for a period of time.

In some embodiments, when DMSO is present, the process further includes, prior to step 1), heating any one or more of b1), b2), and c) at a temperature of from about 40° C. to 50° C. for a period of from about 30 to about 120 minutes. In some embodiments, when DMSO is present, the process further includes, prior to step 1), (1-1) heating b1) and b2) alone (without mixing); (1-2) heating b1), b2), and c) alone (without mixing); (1-3) heating a premixture of b1) and b2); or (1-4) heating a premixture of b1), b2), and c), at a temperature of from about 40° C. to 50° C. for a period of from about 30 to about 120 minutes. In some embodiments, when DMSO is present, the process further includes, prior to step 1), (1-1) heating b1) and b2) alone (without mixing); (1-2) heating b1), b2), and c) alone (without mixing); (1-3) heating a premixture of b1) and b2); or (1-4) heating a premixture of b1), b2), and c), at a temperature of about 45° C. for a period of about 60 minutes. In some embodiments, when DMSO is present, the process further includes, prior to step 1), (1-3) heating a premixture of b1) and b2) or (1-4) heating a premixture of b1), b2), and c), at a temperature of about 45° C. for a period of about 60 minutes.

In some embodiments, when DMSO is present, the process further includes, prior to step 1), forming a premixture of b1) and b2) or a premixture of b1), b2) and c); and mixing the premixture for a period of from about 30 to about 120 minutes at room temperature. In some embodiments, when DMSO is present, the process further includes, prior to step 1), forming a premixture of b1) and b2); and mixing the premixture for a period of from about 30 to about 120 minutes at room temperature. In some embodiments, when DMSO is present, the process further includes, prior to step 1), forming a premixture of b1) and b2); and mixing the premixture for a period of about 60 minutes at room temperature. In some embodiments, when DMSO is present, the process further includes, prior to step 1), forming a premixture of b1), b2) and c); and mixing the premixture for a period of from about 30 to about 120 minutes at room temperature. In some embodiments, when DMSO is present, the process further includes, prior to step 1), forming a premixture of b1), b2), and c); and mixing the premixture for a period of about 60 minutes at room temperature.

In some embodiments, the process further includes:
3) adding a gelling agent to the uniform mixture of step 2) to form a second mixture; and
4) blending the second mixture to form a gel formulation as described herein,
   wherein the gelling agent is defined and described herein.

In some embodiments, the present disclosure provides a process for preparing the formulation (AA); and the process includes:
1) forming a first mixture including:
   a) the compound of formula (I);
   b) a base formulation including:
      b1) DMSO in an amount of from about 1% to about 10% by weight of the base formulation;
      b2) the unsaturated fatty alcohol in an amount of from about 1% to about 10% by weight of the base formulation; and
      b3) C₁₋₃ alkyl-(OCH₂CH₂)₁₋₅—OH, the C₂₋₆ alkylene glycol, the di-(C₂₋₆ alkylene) glycol, and the polyethylene glycol; and
   c) an acid;
2) mixing the first mixture to form a uniform mixture;
3) adding a gelling agent to the uniform mixture of step 2) to form a second mixture; and
4) blending the second mixture to form the formulation (AA),
   wherein
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
the unsaturated fatty alcohol, C₁₋₃ alkyl-(OCH₂CH₂)₁₋₅—OH, the C₂₋₆ alkylene glycol, the di-(C₂₋₆ alkylene) glycol, the polyethylene glycol, and the gelling agent are as described herein.

In some embodiments, the present disclosure provides a process for preparing the formulation (AA1ca); and the process includes:
1) forming a first mixture including:
   a) the compound of formula (I);
   b) a base formulation including:
      b1) DMSO in an amount of about 5% by weight of the base formulation;
      b2) oleyl alcohol in an amount of about 5% by weight of the base formulation; and
      b3) 2-(2-ethoxyethoxy)ethanol, propylene glycol, dipropylene glycol, and PEG400, which are present in an amount of about 25%, about 50%, about 5%, and about 10%, respectively, by weight of the base formulation; and
   c) citric acid in an amount of about 0.05% by weight of the base formulation;
2) mixing the first mixture to form a uniform mixture;
3) adding hydroxypropyl cellulose to the uniform mixture of step 2) to form a second mixture; and
4) blending the second mixture to form the formulation (AA1ca),
   wherein hydroxypropyl cellulose in an amount of from 0.5% to about 2% by weight of the base formulation.

In some embodiments, the present disclosure provides a process for preparing the formulation (AA2ca); and the process includes:
1) forming a first mixture including:
   a) the compound of formula (I);
   b) a base formulation including:
      b1) DMSO in an amount of about 5% by weight of the base formulation;
      b2) oleyl alcohol in an amount of about 3% by weight of the base formulation; and
      b3) 2-(2-ethoxyethoxy)ethanol, propylene glycol, dipropylene glycol, and PEG400, which are present in an amount of about 25%, about 52%, about 5%, and about 10%, respectively, by weight of the base formulation; and
   c) citric acid in an amount of about 0.05% by weight of the base formulation; and
2) mixing the first mixture to form a uniform mixture;
3) adding hydroxypropyl cellulose to the uniform mixture of step 2) to form a second mixture; and
4) blending the second mixture to form the formulation (AA2ca),
   wherein hydroxypropyl cellulose in an amount of from 0.5% to about 2% by weight of the base formulation.

In some embodiments, the present disclosure provides a process for preparing the formulation (AA1aa); and the process includes:
1) forming a first mixture including:
   a) the compound of formula (I);
   b) a base formulation including:
      b1) DMSO in an amount of about 5% by weight of the base formulation;
      b2) oleyl alcohol in an amount of about 5% by weight of the base formulation; and
      b3) 2-(2-ethoxyethoxy)ethanol, propylene glycol, dipropylene glycol, and PEG400, which are present in an amount of about 25%, about 50%, about 5%, and about 10%, respectively, by weight of the base formulation; and
   c) acetic acid in an amount of about 0.05% by weight of the base formulation;
2) mixing the first mixture to form a uniform mixture;
3) adding hydroxypropyl cellulose to the uniform mixture of step 2) to form a second mixture; and
4) blending the second mixture to form the formulation (AA1aa),
   wherein hydroxypropyl cellulose in an amount of from about 0.5% to about 2% by weight of the base formulation.

In some embodiments, the present disclosure provides a process for preparing the formulation (AA2aa); and the process includes:
1) forming a first mixture including:
   a) the compound of formula (I);
   b) a base formulation including:
      b1) DMSO in an amount of about 5% by weight of the base formulation;
      b2) oleyl alcohol in an amount of about 3% by weight of the base formulation; and
      b3) 2-(2-ethoxyethoxy)ethanol, propylene glycol, dipropylene glycol, and PEG400, which are present in an amount of about 25%, about 52%, about 5%, and about 10%, respectively, by weight of the base formulation; and
   c) acetic acid in an amount of about 0.05% by weight of the base formulation;
2) mixing the first mixture to form a uniform mixture;
3) adding hydroxypropyl cellulose to the uniform mixture of step 2) to form a second mixture; and
4) blending the second mixture to form the formulation (AA2aa),
   wherein hydroxypropyl cellulose in an amount of about 0.5% to about 2% by weight of the base formulation.

In some embodiments, the present disclosure provides a process for preparing the formulation (AA1pa); and the process includes:
1) forming a first mixture including:
   a) the compound of formula (I);
   b) a base formulation including:
      b1) DMSO in an amount of about 5% by weight of the base formulation;
      b2) oleyl alcohol in an amount of about 5% by weight of the base formulation; and
      b3) 2-(2-ethoxyethoxy)ethanol, propylene glycol, dipropylene glycol, and PEG400, which are present in an amount of about 25%, about 50%, about 5%, and about 10%, respectively, by weight of the base formulation; and
   c) phosphoric acid in an amount of about 0.005% by weight of the base formulation;
2) mixing the first mixture to form a uniform mixture;
3) adding hydroxypropyl cellulose to the uniform mixture of step 2) to form a second mixture; and
4) blending the second mixture to form the formulation (AA1pa),
   wherein hydroxypropyl cellulose in an amount of from about 0.5% to about 2% by weight of the base formulation.

In some embodiments, the present disclosure provides a process for preparing the formulation (AA2pa); and the process includes:
1) forming a first mixture including:
   a) the compound of formula (I);
   b) a base formulation including:
      b1) DMSO in an amount of about 5% by weight of the base formulation;
      b2) oleyl alcohol in an amount of about 3% by weight of the base formulation; and
      b3) 2-(2-ethoxyethoxy)ethanol, propylene glycol, dipropylene glycol, and PEG400, which are present in an amount of about 25%, about 52%, about 5%, and about 10%, respectively, by weight of the base formulation; and
   c) phosphoric acid in an amount of about 0.005% by weight of the base formulation;
2) mixing the first mixture to form a uniform mixture;
3) adding hydroxypropyl cellulose to the uniform mixture of step 2) to form a second mixture; and
4) blending the second mixture to form the formulation (AA2pa),
   wherein hydroxypropyl cellulose in an amount of from about 0.5% to about 2% by weight of the base formulation.

In some embodiments, the present disclosure provides a process for preparing the formulation (AB); and the process includes:
1) forming a first mixture including:
   a) the compound of formula (I);
   b) a base formulation including:
      b1) DMSO in an amount of from about 5% to about 17% by weight of the base formulation;
      b2) the unsaturated fatty alcohol in an amount of from about 1% to about 10% by weight of the base formulation;
      b3) C₁₋₃ alkyl-(OCH₂CH₂)₁₋₅—OH, the C₂₋₆ alkylene glycol, and the di-(C₂₋₆ alkylene) glycol; and
   c) an acid;
2) mixing the first mixture to form a uniform mixture;
3) adding a gelling agent to the uniform mixture of step 2) to form a second mixture; and
3) blending the second mixture to form the formulation (AB),
   wherein
the acid is i) citric acid in an amount of from about 0.005% to about 0.5% by weight of the base formulation, ii) acetic acid in in an amount of from about 0.005% to about 1% by weight of the base formulation, or iii) phosphoric acid in an amount of from about 0.001% to about 0.03% by weight of the base formulation; and
the unsaturated fatty alcohol, C₁₋₃ alkyl-(OCH₂CH₂)₁₋₅—OH, the C₂₋₆ alkylene glycol, the di-(C₂₋₆ alkylene) glycol, the polyethylene glycol, and the gelling agent are as described herein.

In some embodiments, the present disclosure provides a process for preparing the formulation (AB1ca); and the process includes:
1) forming a first mixture including:
   a) the compound of formula (I);
   b) a base formulation including:
      b1) DMSO in an amount of about 15% by weight of the base formulation;
      b2) oleyl alcohol in an amount of about 5% by weight of the base formulation; and
      b3) 2-(2-ethoxyethoxy)ethanol, propylene glycol, and dipropylene glycol, which are present in an amount of about 25%, about 50%, and about 5%, respectively, by weight of the base formulation; and
   c) citric acid in an amount of about 0.05% by weight of the base formulation;
2) mixing the first mixture to form a uniform mixture;
3) adding hydroxypropyl cellulose to the uniform mixture of step 2) to form a second mixture; and
4) blending the second mixture to form the formulation (AB1ca),
   wherein hydroxypropyl cellulose in an amount of from about 0.5% to about 2% by weight of the base formulation.

In some embodiments, the present disclosure provides a process for preparing the formulation (AB1aa); and the process includes:
1) forming a first mixture including:
   a) the compound of formula (I);
   b) a base formulation including:
      b1) DMSO in an amount of about 15% by weight of the base formulation;
      b2) oleyl alcohol in an amount of about 5% by weight of the base formulation; and
      b3) 2-(2-ethoxyethoxy)ethanol, propylene glycol, and dipropylene glycol, which are present in an amount of about 25%, about 50%, and about 5%, respectively, by weight of the base formulation; and
   c) acetic acid in an amount of about 0.05% by weight of the base formulation;
2) mixing the first mixture to form a uniform mixture;
3) adding hydroxypropyl cellulose to the uniform mixture of step 2) to form a second mixture; and
4) blending the second mixture to form the formulation (AB1aa),
   wherein hydroxypropyl cellulose in an amount of from about 0.5% to about 2% by weight of the base formulation.

In some embodiments, the present disclosure provides a process for preparing the formulation (AB1pa); and the process includes:
1) forming a first mixture including:
   a) the compound of formula (I);
   b) a base formulation including:
      b1) DMSO in an amount of about 15% by weight of the base formulation;
      b2) oleyl alcohol in an amount of about 5% by weight of the base formulation; and
      b3) 2-(2-ethoxyethoxy)ethanol, propylene glycol, and dipropylene glycol, which are present in an amount of about 25%, about 50%, and about 5%, respectively, by weight of the base formulation; and
   c) phosphoric acid in an amount of about 0.005% by weight of the base formulation;
2) mixing the first mixture to form a uniform mixture;
3) adding hydroxypropyl cellulose to the uniform mixture of step 2) to form a second mixture; and
4) blending the second mixture to form the formulation (AB1pa),
   wherein hydroxypropyl cellulose in an amount of from about 0.5% to about 2% by weight of the base formulation.

In some embodiments, the present disclosure provides a process for preparing the formulation (AC); and the process includes:
1) forming a first mixture including:
   a) the compound of formula (I);
   b) a base formulation including:
      b2) the unsaturated fatty alcohol in an amount of from about 1% to about 10% by weight of the base formulation; and
      b3) C₁₋₃ alkyl-(OCH₂CH₂)₁₋₅—OH, the C₂₋₆ alkylene glycol, the di-(C₂₋₆ alkylene) glycol, and the polyethylene glycol; and
   c) an acid;
2) mixing the first mixture to form a uniform mixture;
3) adding a gelling agent to the uniform mixture of step 2) to form a second mixture; and
4) blending the second mixture to form the formulation (AC),
   wherein
the acid is i) citric acid in an amount of from about 0.005% to about 0.5% by weight of the base formulation, ii) the acid is acetic acid in in an amount of from about 0.005% to about 1% by weight of the base formulation, or iii) phosphoric acid in an amount of from about 0.001% to about 0.03% by weight of the base formulation;
the formulation is free of DMSO; and
the unsaturated fatty alcohol, C₁₋₃ alkyl-(OCH₂CH₂)₁₋₅—OH, the C₂₋₆ alkylene glycol, the di-(C₂₋₆ alkylene) glycol, the polyethylene glycol, and the gelling agent are as described herein.

In some embodiments, the present disclosure provides a process for preparing the formulation (AC1ea); and the process includes:
1) forming a first mixture including:
   a) the compound of formula (I);
   b) a base formulation including:
      b2) oleyl alcohol in an amount of about 4.5% by weight of the base formulation; and
      b3) 2-(2-ethoxyethoxy)ethanol, propylene glycol, dipropylene glycol, and PEG400, which are present in an amount of about 23%, about 45.5%, about 4.5%, and about 22.5%, respectively, by weight of the base formulation; and
   c) citric acid in an amount of about 0.05% by weight of the base formulation;
2) mixing the first mixture to form a uniform mixture;
3) adding hydroxypropyl cellulose to the uniform mixture of step 2) to form a second mixture; and
4) blending the second mixture to form the formulation (AC1ea),
   wherein hydroxypropyl cellulose in an amount of from about 0.5% to about 2% by weight of the base formulation; and the formulation is free of DMSO.

In some embodiments, the present disclosure provides a process for preparing the formulation (AC1aa); and the process includes:
1) forming a first mixture including:
   a) the compound of formula (I);
   b) a base formulation including:
      b2) oleyl alcohol in an amount of about 4.5% by weight of the base formulation; and
      b3) 2-(2-ethoxyethoxy)ethanol, propylene glycol, dipropylene glycol, and PEG400, which are present in an amount of about 23%, about 45.5%, about 4.5%, and about 22.5%, respectively, by weight of the base formulation; and
   c) acetic acid in an amount of about 0.05% by weight of the base formulation;
2) mixing the first mixture to form a uniform mixture;
3) adding hydroxypropyl cellulose to the uniform mixture of step 2) to form a second mixture; and
4) blending the second mixture to form the formulation (AC1aa),
   wherein hydroxypropyl cellulose in an amount of from about 0.5% to about 2% by weight of the base formulation; and the formulation is free of DMSO.

In some embodiments, the present disclosure provides a process for preparing the formulation (AC1pa); and the process includes:
1) forming a first mixture including:
   a) the compound of formula (I);
   b) a base formulation including:
      b2) oleyl alcohol in an amount of about 4.5% by weight of the base formulation; and
      b3) 2-(2-ethoxyethoxy)ethanol, propylene glycol, dipropylene glycol, and PEG400, which are present in an amount of about 23%, about 45.5%, about 4.5%, and about 22.5%, respectively, by weight of the base formulation; and
   c) phosphoric acid in an amount of about 0.005% by weight of the base formulation;
2) mixing the first mixture to form a uniform mixture;
3) adding hydroxypropyl cellulose to the uniform mixture of step 2) to form a second mixture; and
4) blending the second mixture to form the formulation (AC1pa),
   wherein hydroxypropyl cellulose in an amount of from about 0.5% to about 2% by weight of the base formulation; and the formulation is free of DMSO.

In some embodiments of any one of processes, the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

The compound of formular (I), a hydrate, a solvate, a pharmaceutically acceptable salt, or a combination thereof, is further described according to Section V. COMPOUNDS and Section IIIA-7.

In some embodiments, the compound of formula (I) in any one of the processes is represented by any one of formulae (IIa), (IIb), (IIc), and (IId), as described herein.

In some embodiments, the compound of formula (I) in any one of the processes is represented by formula (IIa-1a):

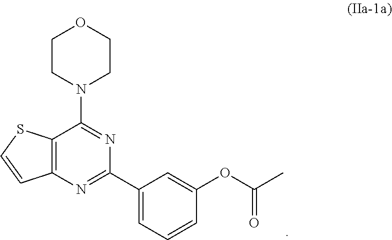 [SMILES: CC(=O)Oc1cccc(-c2nc(N3CCOCC3)c3sccc3n2)c1]

In some embodiments, the present process according to the third aspect is scalable for preparing a formulation according to the first aspect and embodiments as described herein. In some embodiments, the process provides a formulation according to the first aspect on a scale of at least about 1 kilograms (kg), such as at least about 10 kg, about 100 kg, about 1,000 kg, or more. In some embodiments, the process provides a formulation according to the first aspect on a scale of, e.g., from about 1 kg to about 1,000 kg, from about 5 kg to about 1,000 kg, from about 10 kg to about 1,000 kg, from about 100 kg to about 1,000 kg, from about 500 kg to about 1,000 kg, or any range therebetween.

In some embodiments, the present process according to the third aspect provides formulation (AA1ca) on a scale of at least about 1 kg, such as at least about 10 kg, about 100 kg, about 1,000 kg, or more. In some embodiments, the process provides formulation (AA1ca) on a scale of, e.g., from about 1 kg to about 1,000 kg, from about 5 kg to about 1,000 kg, from about 10 kg to about 1,000 kg, from about 100 kg to about 1,000 kg, from about 500 kg to about 1,000 kg, or any range therebetween. In some embodiment, the process provides formulation (AA1ca) on a scale of about 5 kg. In some embodiment, the process provides formulation (AA1ca) on a scale of about 50 kg.

### IVB. Processes for Preparing Formulations Including DMSO

In a fourth aspect, the present disclosure provides a process for preparing a formulation according to the second aspect and embodiments as described herein. The process includes:
1) forming a first mixture including:
   a) a compound having formula (I):

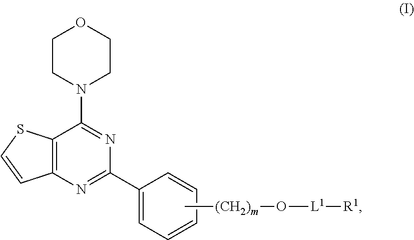

a hydrate, a solvate, a pharmaceutically acceptable salt, or a combination thereof;
wherein:
   subscript m is an integer from 0 to 2;
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) a base formulation including:
   b1) DMSO in an amount of from about 1% to about 20% by weight of the base formulation;
   b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
   b3) one or more solvents including a C₂₋₆ alkylene glycol, a di-(C₂₋₆ alkylene) glycol, or a combination thereof; and
2) sonicating the first mixture to form a second mixture,
   wherein the ratio of DMSO to the combined one or more excipients is at least about 1:1 by weight.

The one or more solvents are defined and described according to Section IIIA-4. In some embodiments, the one or more solvents further include C₁₋₃ alkyl-(OCH₂CH₂)₁₋₅—OH, a polyethylene glycol, or a combination thereof. In some embodiments, the one or more solvents include C₁₋₃ alkyl-(OCH₂CH₂)₁₋₅—OH, a C₂₋₆ alkylene glycol, a di-(C₂₋₆ alkylene) glycol, a polyethylene glycol, or a combination thereof, provided that at least one of a C₂₋₆ alkylene glycol and a di-(C₂₋₆ alkylene) glycol is present. In some embodiments, the one or more solvents include a C₂₋₆ alkylene glycol; and further include C₁₋₃ alkyl-(OCH₂CH₂)₁₋₅—OH, a di-(C₂₋₆ alkylene) glycol, a polyethylene glycol, or a combination thereof.

Embodiments related to C₁₋₃ alkyl-(OCH₂CH₂)₁₋₅—OH, a C₂₋₆ alkylene glycol, a di-(C₂₋₆ alkylene) glycol, and/or a polyethylene glycol are described according to Section IIIA-4.

In some embodiments, the process includes:
1) forming a first mixture including:
   a) the compound of formula (I); and
   b) a base formulation including:
      b1) DMSO in an amount of from about 1% to about 20% by weight of the base formulation;
      b2) an unsaturated fatty alcohol in an amount of no more than about 10% by weight of the base formulation; and
      b3) one or more solvents including C₁₋₃ alkyl-(OCH₂CH₂)₁₋₅—OH, a C₂₋₆ alkylene glycol, a di-(C₂₋₆ alkylene) glycol, a polyethylene glycol, or a combination thereof, provided that at least one of a C₂₋₆ alkylene glycol and a di-(C₂₋₆ alkylene) glycol is present; and
2) sonicating the first mixture to form a second mixture;
   wherein
the ratio of DMSO to the unsaturated fatty alcohol is at least about 1:1 by weight; and
the unsaturated fatty alcohol, C₁₋₃ alkyl-(OCH₂CH₂)₁₋₅—OH, the C₂₋₆ alkylene glycol, the di-(C₂₋₆ alkylene) glycol, and the polyethylene glycol are as defined and described herein.

In some embodiments, the process includes:
1) forming a first mixture including:
   a) the compound of formula (I); and
   b) a base formulation including:
      b1) DMSO in an amount of from about 1% to about 20% by weight of the base formulation;
      b2) an unsaturated fatty alcohol in an amount of no more than about 10% by weight of the base formulation; and
      b3) one or more solvents including a C₂₋₆ alkylene glycol and further including C₁₋₃ alkyl-(OCH₂CH₂)₁₋₅—OH, a di-(C₂₋₆ alkylene) glycol, a polyethylene glycol, or a combination thereof; and
2) sonicating the first mixture to form a second mixture;
   wherein
the ratio of DMSO to the unsaturated fatty alcohol is at least about 1:1 by weight; and
the unsaturated fatty alcohol, C₁₋₃ alkyl-(OCH₂CH₂)₁₋₅—OH, the C₂₋₆ alkylene glycol, the di-(C₂₋₆ alkylene) glycol, and the polyethylene glycol are as defined and described herein.

The duration of sonication can vary according to properties of compounds of formula (I), for example solubility of the compounds, salts of the compounds, and/or forms of the compounds (e.g., hydrate or solvate). The duration of sonication may be substantially shortened (e.g., a few minutes) for certain salts of compounds of formula (I), for example salts derived from inorganic acids (e.g., phosphoric acid) or nontoxic organic acids (e.g., citric acid or acetic acid) as described herein.

In some embodiments, the first mixture is sonicated for one or more minutes. In some embodiments, the first mixture is sonicated for a period of at least about 20 minutes. In some embodiments, the first mixture is sonicated for a period of from about 30 to about 120 minutes. In some embodiments, the first mixture is sonicated for a period of from about 30 to about 60 minutes. In some embodiments, the first mixture is sonicated for a period of about 30 minutes. In some embodiments, the first mixture is sonicated for a period of about 60 minutes.

In some embodiments, the process further includes, prior to step 1), forming a premixture of b1) and b2) and sonicating the premixture of b1) and b2) for a period of from about 10 to about 60 minutes. In some embodiments, the process further includes, prior to step 1), forming a premixture of b1) DMSO and b2) an unsaturated fatty alcohol; and sonicating the premixture of b1) and b2) for a period of from about 10 to about 60 minutes. In some embodiments, the process further includes, prior to step 1), forming a premixture of b1) DMSO and b2) oleyl alcohol; and sonicating the premixture of b1) and b2) for a period of from about 10 to about 60 minutes.

In some embodiments, the process further includes, prior to step 1), forming a premixture of b1) and b2) and heating the premixture of b1) and b2) for a period of from 1 to 10 days at a temperature of about 80° C. In some embodiments, the process further includes, prior to step 1), forming a premixture of b1) DMSO and b2) an unsaturated fatty alcohol; and heating the premixture of b1) and b2) for a period of from 1 to 10 days at a temperature of about 80° C. In some embodiments, the process further includes, prior to step 1), forming a premixture of b1) DMSO and b2) oleyl alcohol; and heating the premixture of b1) and b2) for a period of from 1 to 10 days at a temperature of about 80° C.

In some embodiments, the process further includes:
3) adding a gelling agent to the second mixture of step 2) to form a third mixture; and
4) blending the third mixture to form a gel formulation as described herein,
   wherein the gelling agent is defined and described herein.

In some embodiments, the present disclosure provides a process for preparing the formulation (BA), the process including:
1) forming a first mixture including:
   a) the compound of formula (I); and
   b) a base formulation including:
      b1) DMSO in an amount of from about 1% to about 10% by weight of the base formulation;
      b2) the unsaturated fatty alcohol in an amount of from about 1% to about 10% by weight of the base formulation; and
      b3) C₁₋₃ alkyl-(OCH₂CH₂)₁₋₅—OH, the C₂₋₆ alkylene glycol, the di-(C₂₋₆ alkylene) glycol, and the polyethylene glycol;
2) sonicating the first mixture to form a second mixture;
3) adding a gelling agent to the second mixture of step 2) to form a third mixture; and
4) blending the third mixture to form the formulation (BA),
   wherein the unsaturated fatty alcohol, C₁₋₃ alkyl-(OCH₂CH₂)₁₋₅—OH, the C₂₋₆ alkylene glycol, the di-(C₂₋₆ alkylene) glycol, the polyethylene glycol, and the gelling agent are as defined and described herein.

In some embodiments, the present disclosure provides a process for preparing the formulation (BA1), the process including:
1) forming a first mixture including:
   a) the compound of formula (I); and
   b) a base formulation including:
      b1) DMSO in an amount of about 5% by weight of the base formulation;
      b2) oleyl alcohol in an amount of about 5% by weight of the base formulation; and
      b3) 2-(2-ethoxyethoxy)ethanol, propylene glycol, dipropylene glycol, and PEG400, which are present in an amount of about 25%, about 50%, about 5%, and about 10%, respectively, by weight of the base formulation;
2) sonicating the first mixture to form a second mixture;
3) adding hydroxypropyl cellulose to the second mixture of step 2) to form a third mixture; and
4) blending the third mixture to form the formulation (BA1),
   wherein hydroxypropyl cellulose is in an amount of from about 0.5% to about 2% by weight of the base formulation.

In some embodiments, the present disclosure provides a process for preparing the formulation (BA2), the process including:
1) forming a first mixture including:
   a) the compound of formula (I); and
   b) a base formulation including:
      b1) DMSO in an amount of about 5% by weight of the base formulation;
      b2) oleyl alcohol in an amount of about 3% by weight of the base formulation; and
      b3) 2-(2-ethoxyethoxy)ethanol, propylene glycol, dipropylene glycol, and PEG400, which are present in an amount of about 25%, about 52%, about 5%, and about 10%, respectively, by weight of the base formulation;
2) sonicating the first mixture to form a second mixture;
3) adding hydroxypropyl cellulose to the second mixture of step 2) to form a third mixture; and
4) blending the third mixture to form the formulation (B A2),
   wherein hydroxypropyl cellulose is in an amount of from about 0.5% to about 2% by weight of the base formulation.

In some embodiments, the present disclosure provides a process for preparing the formulation (BB). The process includes:
1) forming a first mixture including:
   a) the compound of formula (I); and
   b) a base formulation including:
      b1) DMSO in an amount of from about 5% to about 17% by weight of the base formulation;
      b2) the unsaturated fatty alcohol in an amount of from about 1% to about 10% by weight of the base formulation; and
      b3) C₁₋₃ alkyl-(OCH₂CH₂)₁₋₅—OH, the C₂₋₆ alkylene glycol, and the di-(C₂₋₆ alkylene) glycol;
2) sonicating the first mixture to form a second mixture;
3) adding a gelling agent to the second mixture of step 2) to form a third mixture; and
4) blending the third mixture to form the formulation (BB),
   wherein the unsaturated fatty alcohol, C₁₋₃ alkyl-(OCH₂CH₂)₁₋₅—OH, the C₂₋₆ alkylene glycol, the di-(C₂₋₆ alkylene) glycol, and the gelling agent are as described herein.

In some embodiments, the present disclosure provides a process for preparing the formulation (BB1). The process includes:
1) forming a first mixture including:
   a) the compound of formula (I); and
   b) a base formulation including:
      b1) DMSO in an amount of about 15% by weight of the base formulation;
      b2) oleyl alcohol in an amount of about 5% by weight of the base formulation; and
      b3) 2-(2-ethoxyethoxy)ethanol, propylene glycol, and dipropylene glycol, which are present in an amount of about 25%, about 50%, and about 5%, respectively, by weight of the base formulation;
2) sonicating the first mixture to form a second mixture;
3) adding hydroxypropyl cellulose to the second mixture of step 2) to form a third mixture; and
4) blending the third mixture to form the formulation (BB1),
   wherein hydroxypropyl cellulose is in an amount of from about 0.5% to about 2% by weight of the base formulation.

In some embodiments of any one of processes, the hydroxypropyl cellulose has an average molecular weight of from about 700,000 Da to about 1,150,000 Da. In some embodiments, the hydroxypropyl cellulose has an average molecular weight of about 700,000 Da (e.g., Nisso M). In some embodiments, the hydroxypropyl cellulose has an average molecular weight of about 850,000 Da (e.g., Klucel MF). In some embodiments, the hydroxypropyl cellulose has an average molecular weight of about 1,000,000 Da (e.g., Nisso H). In some embodiments, the hydroxypropyl cellulose has an average molecular weight of about 1,150,000 Da (e.g., Klucel HF).

The compound of formular (I), a hydrate, a solvate, a pharmaceutically acceptable salt, or a combination thereof, is further described according to Section V. COMPOUNDS and Section IIIA-7.

In some embodiments, the compound of formula (I) in any one of the processes is represented by any one of formulae (IIa), (IIb), (IIc), and (IId), as described herein.

In some embodiments, the compound of formula (I) in any one of the processes is represented by formula (IIa-1a):

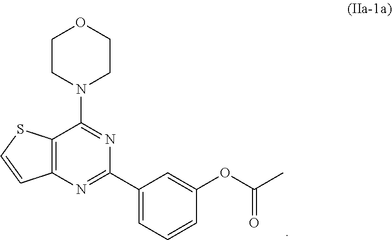 [SMILES: CC(=O)Oc1cccc(-c2nc(N3CCOCC3)c3sccc3n2)c1]

### IVC. Formulations Prepared by Processes

In a fifth aspect, the present disclosure provides a formulation, prepared by a process according to the third aspect and embodiments as described herein.

In some embodiments, the formulation, prepared by the process according to the third aspect and embodiments as described herein, is any one of formulations (AA), (AA1ca), (AA2ca), (AA1aa), (AA2aa), (AA1pa), (AA2pa), (AB), (AB1ca), (AB1aa), (AB1pa), (AC), (AC1ea), (AC1aa), and (AC1pa).

In a sixth aspect, the present disclosure provides a formulation, prepared by a process according to the fourth aspect and embodiments as described herein.

In some embodiments, the formulation, prepared by the process according to the fourth aspect and embodiments as described herein, is any one of formulations (BA), (BA-1), (BA1), (BA1-1), (BA2), (BA2-1), (BB), (BB-1), (BB1), and (BB1-1) to (BB1-6).

### V. Compounds

The present disclosure provides a compound useful in formulations, compositions, processes for preparing formulations, and methods for treating a vascular malformation as described herein, wherein the compound is represented by formula (I):

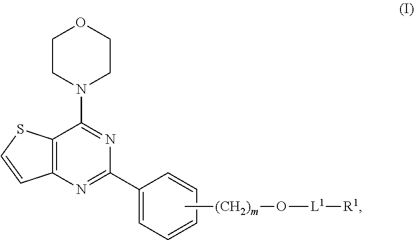

a hydrate, a solvate, a pharmaceutically acceptable salt, or a combination thereof,
wherein:
subscript m is an integer from 0 to 2;
L¹ is —C(O)—, —C(O)O—, —C(O)S—, or —C(O)NH—; and
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl.

In some embodiments, subscript m is 0 or 1. In some embodiments, subscript m is 1. In some embodiments, subscript m is 0.

In some embodiments, subscript m is 0 and the compound is represented by formula (II):

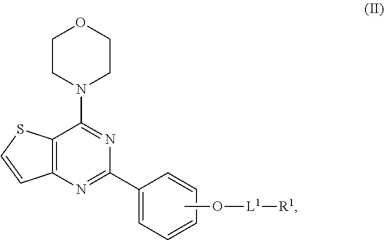

wherein L¹ and R¹ are as defined herein in any aspect or embodiments described herein.

In some embodiments, subscript m is 1 and the compound is represented by formula (III):

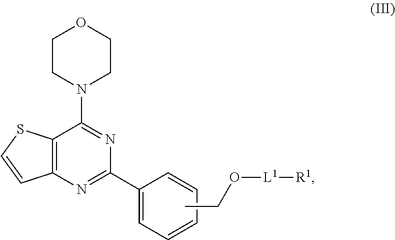

wherein L¹ and R¹ are as defined herein in any aspect or embodiments described herein.

In some embodiments of any one of formulae (I), (II), and (III), L¹ is —C(O)—. In some embodiments, L¹ is —C(O)O—. In some embodiments, L¹ is —C(O)NH—. In some embodiments, L¹ is —C(O)S—.

In some embodiments of formula (II), L¹ is —C(O)— and the compound is represented by formula (IIa):

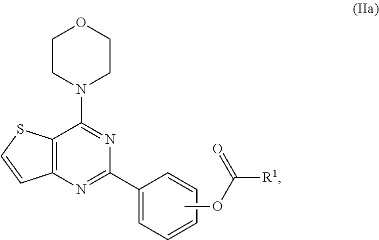

wherein R¹ is as defined herein in any aspect or embodiments described herein.

In some embodiments of formula (II), the compound is represented by any one of formulae (IIb), (IIc), and (IId):

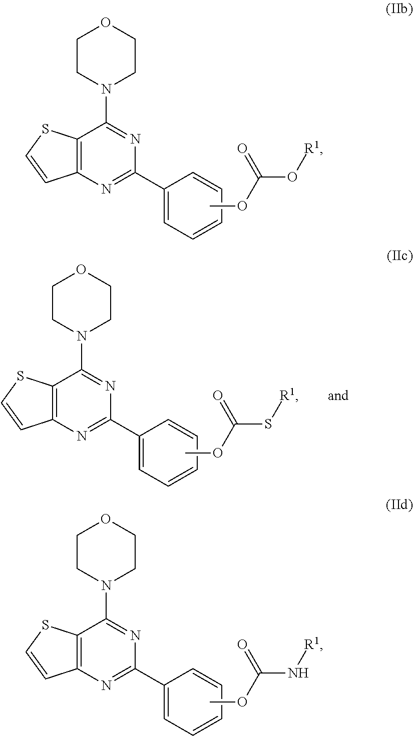

wherein R¹ is as defined herein in any aspect or embodiments described herein.

In some embodiments of formula (III), L¹ is —C(O)— and the compound is represented by formula (IIIa):

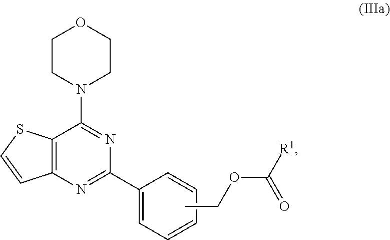

wherein R¹ is as defined herein in any aspect or embodiments described herein.

In some embodiments of formula (III), the compound is represented by any one of formulae (IIIb), (IIIc), and (IIId):

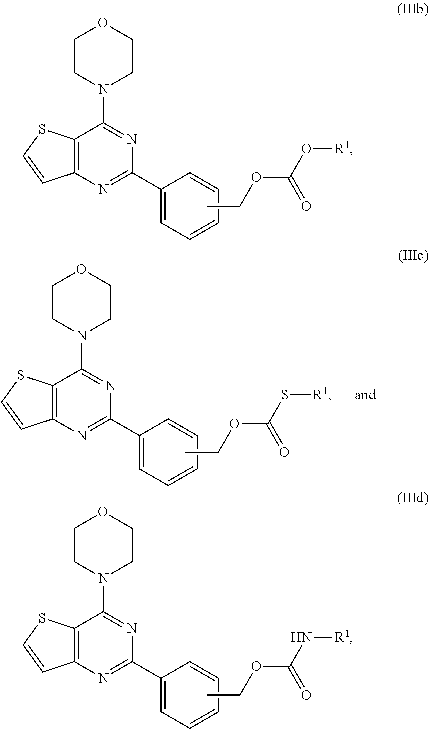

wherein R¹ is as defined herein in any aspect or embodiments described herein.

With reference to any one of formulae (I) to (III), (IIa) to (IId), and (IIIa) to (IIId), in some embodiments, R¹ is C₁₋₆ alkyl, C₂₋₆ alkenyl, C₆₋₁₀ aryl, C₆₋₁₀ aryl-C₁₋₆ alkyl, or C₆₋₁₀ aryl-C₂₋₆ alkenyl.

In some embodiments of any one of formulae (I) to (III), (IIa) to (IId), and (IIIa) to (IIId), R¹ is C₁₋₆ alkyl. In some embodiments, R¹ is methyl, ethyl, propyl, isopropyl, butyl, isobutyl, sec-butyl, tert-butyl, pentyl, isopentyl, or hexyl. In some embodiments, R¹ is methyl. In some embodiments, R¹ is ethyl.

In some embodiments of any one of formulae (I) to (III), (IIa) to (IId), and (IIIa) to (IIId), R¹ is C₂₋₆ alkenyl. In some embodiments, R¹ is vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. In some embodiments, R¹ is propenyl.

In some embodiments of any one of formulae (I) to (III), (IIa) to (IId), and (IIIa) to (IIId), R¹ is C₆₋₁₀ aryl-C₂₋₆ alkenyl. In some embodiments, R¹ is phenyl-C₂₋₆ alkenyl. In some embodiments, R¹ is phenyl-CH═CH—.

Exemplified compounds of formula (I) are listed in Table 1.

TABLE 1
Compounds of formula (I)

| No. | Structure |
|---|---|
| 1.002 | 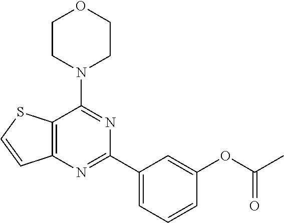 [SMILES: CC(=O)Oc1cccc(-c2nc(N3CCOCC3)c3sccc3n2)c1] |
| 1.003 | 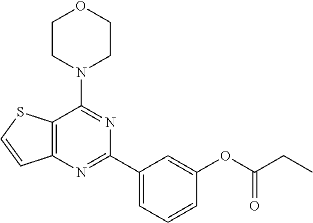 [SMILES: CCC(=O)Oc1cccc(-c2nc(N3CCOCC3)c3sccc3n2)c1] |
| 1.004 | 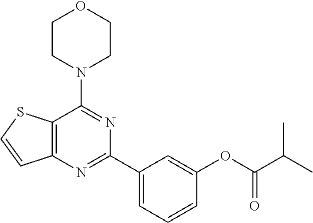 [SMILES: CC(C)C(=O)Oc1cccc(-c2nc(N3CCOCC3)c3sccc3n2)c1] |
| 1.005 | 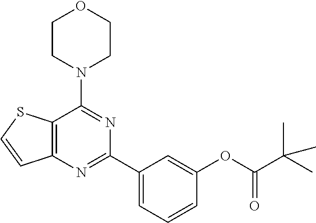 [SMILES: CC(C)(C)C(=O)Oc1cccc(-c2nc(N3CCOCC3)c3sccc3n2)c1] |
| 1.006 | 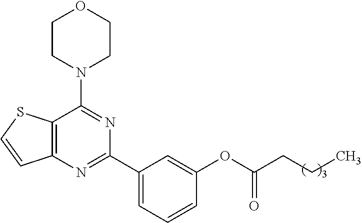 |
| 1.012 | 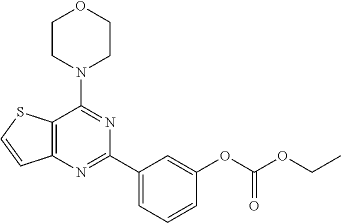 [SMILES: CCOC(=O)Oc1cccc(-c2nc(N3CCOCC3)c3sccc3n2)c1] |
| 1.013 | 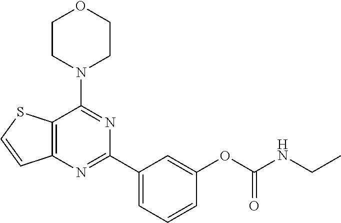 [SMILES: CCNC(=O)Oc1cccc(-c2nc(N3CCOCC3)c3sccc3n2)c1] |
| 1.014 | 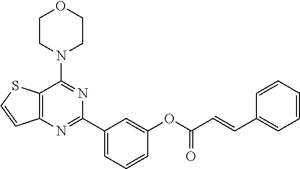 [SMILES: O=C(/C=C/c1ccccc1)Oc1cccc(-c2nc(N3CCOCC3)c3sccc3n2)c1] |
| 1.015 | 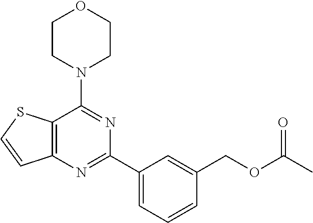 [SMILES: CC(=O)OCc1cccc(-c2nc(N3CCOCC3)c3sccc3n2)c1] |

In some embodiments, the compound of any one of formulae (I), (II), and (IIa) has formula (IIa-1a) (i.e., Compound 1.002):

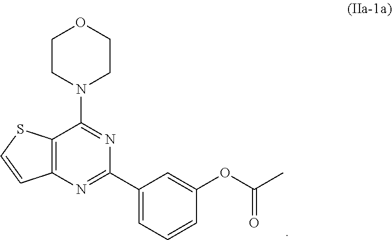 [SMILES: CC(=O)Oc1cccc(-c2nc(N3CCOCC3)c3sccc3n2)c1]

In some embodiments, the compound of any one of formulae (I), (III), and (IIIa) has formula (IIIa-1a) (i.e., Compound 1.015):

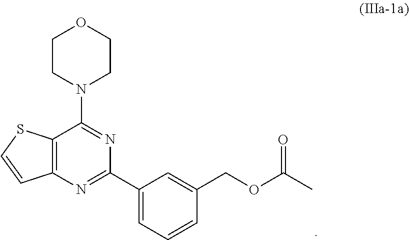 [SMILES: CC(=O)OCc1cccc(-c2nc(N3CCOCC3)c3sccc3n2)c1]

The compounds of formula (I) or Table 1 can be prepared according to synthetic methods described in International Patent Application No. PCT/US2020/016876, which is incorporated herein in its entirety for all purposes.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures), succinates, benzoates and salts with amino acids such as glutamic acid. These salts may be prepared by various methods. When compounds of the present disclosure contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

Other salts include acid salts of the compounds used in the methods of the present disclosure. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in, e.g., Remington's Pharmaceutical Sciences, 17th Edition, 1985, Mack Publishing Company, Easton, Pa.

Pharmaceutically acceptable salts include salts of an active compounds that are prepared with relatively nontoxic acids, depending on the particular substituents found on the compounds described herein. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19).

The neutral forms of the compounds may be regenerated by contacting the acid salts with a base and isolating the parent compound. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of the present disclosure possess double bonds; tautomers, geometric isomers and individual isomers are encompassed within the scope of the present disclosure. The compounds of the present disclosure do not include those which are known in art to be too unstable to synthesize and/or isolate.

Isomers include compounds having the same number and kind of atoms, and hence the same molecular weight, but differing in respect to the structural arrangement or configuration of the atoms. Isomers including stereoisomers (e.g., enantiomers, diastereomers, atropisomers, etc.) of compounds described herein are encompassed within the scope of the present disclosure.

Certain compounds of this disclosure may exist in tautomeric forms, all such tautomeric forms of the compounds being within the scope of the disclosure. Tautomer refers to one of two or more structural isomers which exist in equilibrium and which are readily converted from one isomeric form to another.

Unless otherwise stated, the compounds of the present disclosure may also contain unnatural proportions of atomic isotopes at one or more of the atoms that constitute such compounds. For example, the compounds of the present disclosure may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present disclosure, whether radioactive or not, are encompassed within the scope of the present disclosure.

### VI. Methods

In a seventh aspect, the present disclosure provides a method of treating a vascular malformation through inhibiting phosphoinositide-3-kinase (PI3K) with the formulation including the compound of formula (I), as described herein.

In some embodiments, the formulation is prepared by a process as defined and described herein (e.g., the process according to the third or fourth aspect and embodiments as described herein).

The term “vascular malformation,” as used herein, refers to a non-malignant, congenital abnormality of blood and/or lymph vessels that may be apparent at birth or alternatively may not be apparent at birth and may present weeks, months, or years later. In some cases, a vascular malformation may develop in response to an injury or physiological change such as pregnancy or puberty. Vascular malformations may be of various types including, for example, port-wine stains (capillary vascular malformations), spider angiomas, venous malformations, lymphatic malformations, arteriovenous malformations, pyogenic granulomas (lobular capillary hemangiomas), hemangiomas, pigmented skin lesions, angiofibromas, and glomangiomas. In some embodiments, the vascular malformation is not a hemangioma. In some embodiments, a vascular malformation is characterized by the presence of a single endothelial layer forming distended blood vessels of variable diameter that are surrounded by a disorganized mural cell layer containing both smooth muscle cells and pericytes. Additional details of vascular malformations are described elsewhere herein.

In some embodiments, the vascular malformation is or includes a venous malformation, an arterial malformation, an arteriovenous malformation or a lymphatic vessel malformation. In some embodiments, the subject suffers from multiple vascular malformations. In some embodiments, the subject suffers from a venous malformation.

Vascular malformations may be located in or adjacent to diverse areas of the body, including but not limited to the central nervous system (brain, spinal cord), skin, eye (including but not limited to the retina), ear, (facial) sinus, organs such as the lung, heart, liver, gallbladder, spleen, gastrointestinal system (esophagus, stomach, duodenum, intestine, colon, rectum), pancreas, kidney, bladder, ovary, testicle, joints, nose, lips, etc. In some embodiments, a vascular malformation (e.g., venous malformation) or portion thereof is apparent upon visual inspection (e.g., the vascular malformation or portion thereof is apparent from observation of a surface of the subject, such as the skin of a subject). In some embodiments, a vascular malformation (e.g., venous malformation) or portion thereof is not apparent upon visual inspection (e.g., the vascular malformation or portion thereof is not apparent from observation of a surface of the subject, such as the skin of a subject). In some embodiments, a vascular malformation or portion thereof is accessible at the surface of the subject, such as at the skin of the subject. In some embodiments, a vascular malformation or portion thereof is not accessible at the surface of the subject, such as at the skin of the subject.

In some embodiments, the subject suffers from a malignancy (e.g., a malignant vascular tumor or vascular anomaly). In some embodiments, the subject is not known to suffer from a malignancy (e.g., a malignant vascular tumor or vascular anomaly).

In some embodiments, the subject suffers from a multisystem genetic disorder. In some embodiments, the subject is not known to suffer from a multisystem genetic disorder. In some embodiments, the subject has been diagnosed with or is suspected of having a disorder associated with vascular malformations, such as a disorder selected from Klippel-Trenaunay Syndrome; Parkes-Weber Syndrome; Blue Rubbert Bleb Nevus Syndrome (BRBNS; also referred to as Bean Syndrome); Congenital Lipomatus Overgrowth, Vascular Malformations, Epidermal Nevi and Spine Deformities (CLOVES); Hereditary Hermorrhagic Telangiectasias (HHT; also referred to as Osler-Webe-Rendu Syndrome); Proteus Syndrome; and venolymphatic malformations including angiokeratoma. In some embodiments, the subject has been diagnosed with or is suspected of having another disorder or condition associated with vascular malformations. In some embodiments, the subject has previously undergone treatment for a condition associated with vascular malformations.

In some embodiments, the subject has a single vascular malformation. In some embodiments, the subject has one or more vascular malformations, or a system of vascular malformations. In some embodiments, a vascular malformation impacts one or more organs, tissues, muscles, joints, or bones in its vicinity. In some embodiments, the subject has one or more symptoms or conditions selected from a lump, birthmark, pigmented skin lesion, fluid leakage, pain, cardiovascular stress, bleeding, clotting disorder, organ damage, a fluid-filled pocket or cyst, and infection, which one or more symptoms or conditions can be associated with a vascular malformation.

In some embodiments, the subject suffers from at least one vascular malformation, the surgical or other invasive treatment of which would be high-risk. Such a vascular malformation may be at least partially located in an area that is, because of its location, difficult to access without substantial risk of morbidity or mortality (for example, but not limited to, malformations in the brain, e.g., the brainstem), and/or may be in a weakened subject where surgery or other invasive treatment is contraindicated. Further, if there are multiple lesions, medical treatment may be preferable over surgical options because of aggregate risk, efficiency, or because of risk of recurrence.

In some embodiments, the subject is at risk for occurrence or recurrence of a vascular malformation, for example because of heredity and/or a previously existing lesion.

In some embodiments, a formulation (e.g., topical formulation) provided herein is administered to the subject topically in the area of the vascular malformation, such as to a surface of the subject (e.g., the skin of the subject). In some embodiments, a formulation (e.g., topical formulation) provided herein is administered to the subject topically as a foam, a lotion, a spray, an aerosol, an ointment, a cream, a gel, a paste, a patch, or an in-situ patch.

After administration (e.g., topical delivery) of a formulation comprising a compound of formula (I), it is believed that the compound of formula (I) is converted to a corresponding compound of formula (IV), as shown below:

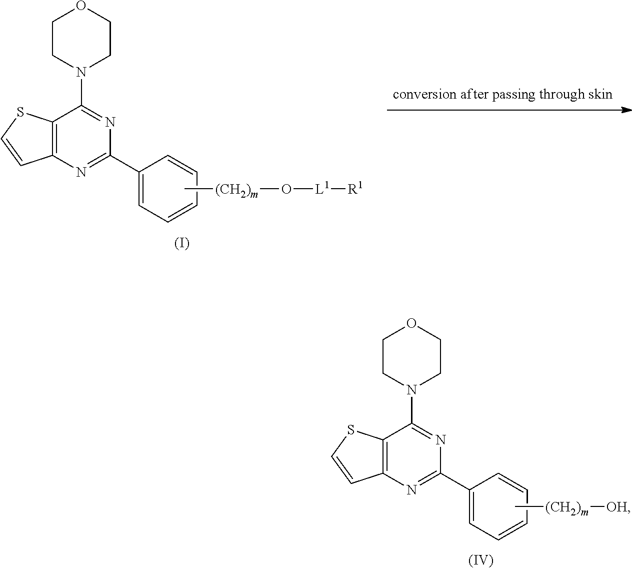

wherein subscript m, L¹, and R¹ are as defined and described herein.

In some embodiments, a method of the present disclosure includes topical administration to a subject (e.g., to a surface of a subject, such as to the skin of the subject) of a formulation (e.g., topical formulation) provided herein that comprises a compound of formula (I), which compound or a metabolite or hydrolysis product thereof (e.g., a corresponding compound of formula (IV)) is capable of at least partially inhibiting one or more of the phosphoinositide 3-kinase enzymes that are part of the PI3K/AKT pathway, thereby providing a beneficial therapeutic effect for the treatment of a vascular malformation (e.g., venous malformation) or a symptom or condition associated therewith (e.g., as described herein).

In some embodiments, a method of the present disclosure includes topical administration to a subject (e.g., to a surface of a subject, such as to the skin of the subject) of a formulation (e.g., topical formulation) provided herein that comprises a compound of formula (I), which compound of formula (I), upon administration to the subject, is at least partially converted to a corresponding compound of formula (IV), which compound of formula (IV) is capable of at least partially inhibiting one or more of the phosphoinositide 3-kinase enzymes that are part of the PI3K/AKT pathway, thereby providing a beneficial therapeutic effect for the treatment of a vascular malformation (e.g., venous malformation) or a symptom or condition associated therewith (e.g., as described herein).

In some embodiments of the methods described herein, the compound of formula (I) is substantially converted to the compound of formula (IV) upon administration to the subject. In some embodiments, at least about 25% of the compound of formula (I), such as at least about 30%, 35%, 40%, 45%, 50%, 55%, 60%, 65%, 70%, 75%, 80%, 85%, 90%, 95%, 96%, 97%, 98%, 99%, or more, is converted to the compound of formula (IV) upon administration to the subject, such as by hydrolysis. In some embodiments, the conversion of the compound having formula (I) to a corresponding compound having formula (IV) after administration to the skin of the subject is at least about 50% over a period of about 24 hours. In some embodiments, the conversion of the compound having formula (I) to a corresponding compound having formula (IV) after administration to the skin of the subject is from about 50% to about 99%, from about 60% to about 90%, or from about 70% to about 90% over a period of about 24 hours. In some embodiments, the conversion of the compound having formula (I) to a corresponding compound having formula (IV) after administration to the skin of the subject is about 85% over a period of about 24 hours.

In some embodiments of the methods provided herein, when a formulation comprising a compound of formula (I) is delivered to a subject by a non-topical route, the conversion of the compound of formula (I) to a corresponding compound of formula (IV) in a subject is at least about 50% over a period of about one (1) hour. In some embodiments, the conversion of the compound of formula (I) to a corresponding compound of formula (IV) in a subject is from about 50% to about 99%, from about 60% to about 90%, or from about 70% to about 90% over a period of about one (1) hour. In some embodiments, the conversion of the compound of formula (I) to a corresponding compound of formula (IV) in a subject is about 85% over a period of about one (1) hour.

In some embodiments of the methods provided herein, the compound of formula (I) included in a formulation (e.g., topical formulation) is any one of formulae (II), (IIa), (IIb), (IIc), (IId), (IIa-1), (IIa-1a), (IIIa), (IIIb), (IIIc), (IIId), (IIIa-1), and (IIIa-1a). In some embodiments, the compound of formula (I) in the formulation used in the method is 3-(4-morpholinothieno[3,2-d]pyrimidin-2-yl)phenyl acetate (i.e., formula (IIa-1a)). In some embodiments, the compound of formula (I) in the formulation or used in the method is 3-(4-morpholinothieno[3,2-d]pyrimidin-2-yl)benzyl acetate (i.e., formula (IIIa-1a)).

In some embodiments of the methods provided herein, the compound of formula (I) included in a formulation (e.g., topical formulation) is represented by formula (IIa):

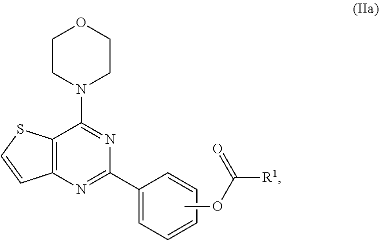

wherein R¹ is as defined and described herein.

In some embodiments of the methods provided herein, the compound of formula (I) included in a formulation (e.g., topical formulation) is represented by any one of formulae (IIb), (IIc), and (IId):

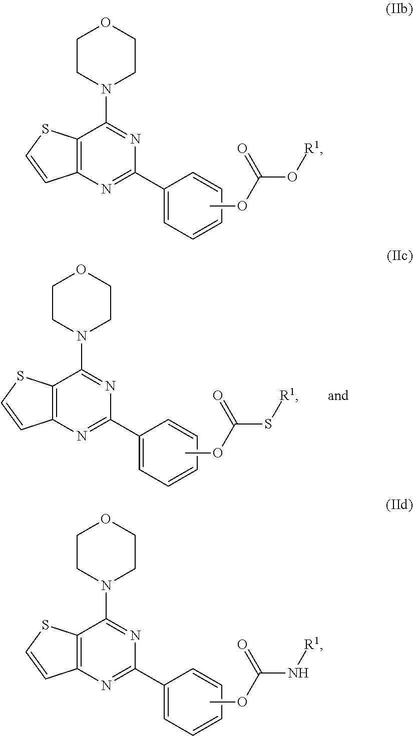

wherein R¹ is as defined herein in any aspect or embodiments described herein.

In some embodiments of the methods provided herein, the compound of formula (I) included in a formulation (e.g., topical formulation) is represented by formula (IIa-1a):

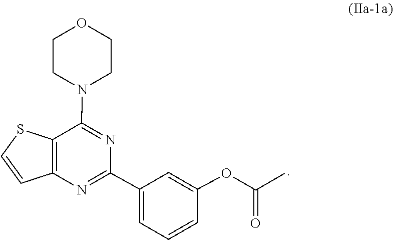 [SMILES: CC(=O)Oc1cccc(-c2nc(N3CCOCC3)c3sccc3n2)c1]

After topical delivery to a subject (e.g., to the skin of a subject), it is believed that the compound of formula (IIa-1) is converted to a corresponding compound of formula (IVa), as shown below:

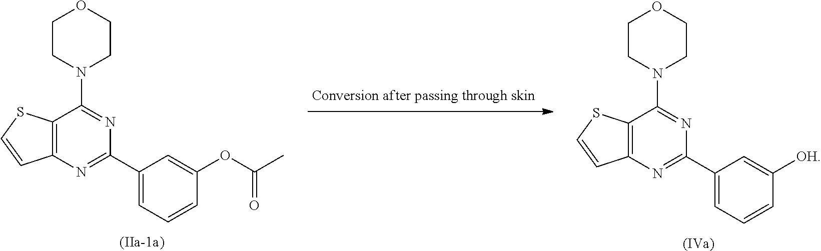

In some embodiments of the methods provided herein, the method includes topical administration to a subject (e.g., to the surface of the subject, such as to the skin of the subject) of a formulation provided herein that include the compound 3-(4-morpholinothieno[3,2-d]pyrimidin-2-yl)phenyl acetate (i.e., formula (IIa-1a)), which compound is converted to 3-(4-morpholinothieno[3,2-d]pyrimidin-2-yl)phenol (i.e., formula (IVa)) upon administration to the subject, which at least partially inhibits the PI3K/AKT pathway, thereby treating a vascular malformation or symptom or condition thereof in the subject.

In some embodiments of the methods provided herein, the method includes topical administration to a subject (e.g., to the surface of the subject, such as to the skin of the subject) of a formulation provided herein that includes the compound 3-(4-morpholinothieno[3,2-d]pyrimidin-2-yl)benzyl acetate (i.e., formula (IIIa-1a)), which compound is converted to (3-(4-morpholinothieno[3,2-d]pyrimidin-2-yl)phenyl)methanol (i.e., formula (IVb)) upon administration to the subject, which at least partially inhibits the PI3K/AKT pathway, thereby treating a vascular malformation or symptom or condition thereof in the subject.

In some embodiments of the methods provided herein, a compound of formula (IV) that is provided upon administration of a formulation comprising a compound of formula (I) to a subject (e.g., to the surface of the subject, such as to the skin of the subject) is a selective inhibitor of the alpha isoform (p110α) of PI3K.

The treatment methods of the disclosure may be administered alone or in conjunction with another form of pharmaceutical and/or surgical therapy. Non-limiting examples of pharmaceutical treatments and/or agents include, but are not limited, to treatment with one or more of: an anti-angiogenic agent, a steroid, an mTOR inhibitor, a beta-blocker (e.g., propranolol), and/or an agent that reduces blood pressure. In certain embodiments, “in conjunction with,” means that an inhibitor of the PI3K/AKT pathway and another pharmaceutical agent, e.g., an mTOR inhibitor, are administered to a subject as part of a treatment regimen or plan. In certain embodiments, being used in conjunction does not require that the PI3K/AKT pathway inhibitor and the pharmaceutical agent are physically combined prior to administration or that they be administered over the same time frame.

### VII. Compositions

In an eighth aspect, the present disclosure provides a composition including:
a) a compound comprising one or more groups selected from the group consisting of an ester, a lactone, an amide, a lactam, a carbonate, a thiocarbonate, and a carbamate, provided that the amide is other than C(O)NH₂;
b) DMSO in an amount of from about 0% to about 30% by weight;
c) one or more excipients selected from the group consisting of an unsaturated fatty alcohol, an unsaturated fatty acid, an unsaturated fatty ester, an unsaturated fatty ether, and a combination thereof; and
d) optionally one or more additional excipients; and
e) an acid in an amount of no more than about 1% by weight,
   wherein
the one or more excipients are in an amount of no more than about 10% by weight; and
the compound maintains a relative purity of at least about 90% over a period of about 10 days at a temperature of 80° C. (±2° C.) or over a period of about 6 months at a temperature of 40° C. (±2° C.) and a relative humidity of 75% (±5%).

In a ninth aspect, the present disclosure provides a composition including:
a) a compound comprising one or more groups selected from the group consisting of an ester, a lactone, an amide, a lactam, a carbonate, a thiocarbonate, and a carbamate, provided that the amide is other than C(O)NH₂;
b) DMSO in an amount of from about 0.1% to about 30% by weight;
c) one or more excipients selected from the group consisting of an unsaturated fatty alcohol, an unsaturated fatty acid, an unsaturated fatty ester, an unsaturated fatty ether, and a combination thereof; and
d) optionally one or more additional excipients,
   wherein
the one or more excipients are in an amount of no more than about 10% by weight;
the ratio of DMSO to the combined one or more excipients is at least about 1:1 by weight; and
the compound maintains a relative purity of at least about 90% over a period of about 10 days at a temperature of 80° C. (±2° C.) or over a period of about 6 months at a temperature of 40° C. (±2° C.) and a relative humidity of 75% (±5%).

In some embodiments, the compound includes one or more groups selected from an ester, a lactone, a carbonate, a thiocarbonate, a carbamate, or a combination thereof. In some embodiments, the compound includes one or more groups selected from an ester, a lactone, a carbonate, or a combination thereof. In some embodiments, the compound includes an ester or a carbonate. In some embodiments, the compound includes an ester. In some embodiments, the compound includes acetate.

With reference to the composition according to the eighth aspect, in some embodiments, DMSO is absent from (e.g., not included in) the composition. In some embodiments, when DMSO is absent, the composition includes an acid in an amount of no more than about 1% by weight, thereby reducing the hydrolysis of an ester, a lactone, a carbonate, a thiocarbonate, or a carbamate. In some embodiments, when DMSO is absent, the composition includes an acid in an amount of no more than about 1% by weight, thereby reducing the hydrolysis of an ester or a carbonate. In some embodiments, when DMSO is absent, the composition includes an acid in an amount of no more than about 1% by weight, thereby reducing the hydrolysis of an ester. In some embodiments, when DMSO is absent, the composition includes an acid in an amount of no more than about 1% by weight, thereby reducing the hydrolysis of an aryl ester. In some embodiments, when DMSO is absent, the composition includes an acid in an amount of no more than about 1% by weight, thereby reducing the hydrolysis of an aryl acetate.

With reference to the composition according to the eighth aspect, in some embodiments, the composition includes DMSO in an amount of from about 0.1% to about 30% by weight and an acid in an amount of no more than about 1% by weight, thereby reducing the hydrolysis of an ester, a lactone, a carbonate, a thiocarbonate, or a carbamate. In some embodiments, the composition includes DMSO in an amount of from about 0.1% to about 30% by weight and an acid in an amount of no more than about 1% by weight, thereby reducing the hydrolysis of an ester or a carbonate. In some embodiments, the composition includes DMSO in an amount of from about 0.1% to about 30% by weight and an acid in an amount of no more than about 1% by weight, thereby reducing the hydrolysis of an ester. In some embodiments, the composition includes DMSO in an amount of from about 0.1% to about 30% by weight and an acid in an amount of no more than about 1% by weight, thereby reducing the hydrolysis of an aryl ester. In some embodiments, the composition includes DMSO in an amount of from about 0.1% to about 30% by weight and an acid in an amount of no more than about 1% by weight, thereby reducing the hydrolysis of an aryl acetate.

With respect to the composition according to the eighth aspect, in some embodiments, DMSO is absent. In some embodiments, DMSO is present in an amount of from about 0.1% to about 20% by weight. In some embodiments, DMSO is present in an amount of from about 1% to about 15% by weight of the base formulation, as defined and described herein (see Section IIIA-2. DMSO under Section III. Formulations).

With reference to the composition according to the eighth aspect, the acid can be any acid that is capable of protonating hydroperoxyl species without causing additional hydrolysis of the compound under an acidic condition. As described herein, the hydroperoxyl species are the deprotonated species of peroxides present in one or more excipients including an unsaturated fatty alcohol (e.g., oleyl alcohol), an unsaturated fatty acid (e.g., oleic acid), or an unsaturated ester or ether (e.g., containing the sidechain of oleyl alcohol and/or oleic acid). Suitable acids include organic acids having a pKa of from about 3 to about 6 and inorganic acids as defined and described herein (see Section IIIA-3. Acid under Section III. Formulations).

In some embodiments, the acid is an organic acid having a pKa of from about 3 to about 6, as defined and described herein. In some embodiments, the acid is an inorganic acid selected from the group consisting of hydrochloric acid (HCl), boric acid (H₃BO₃), sulfuric acid (H₂SO₄), carbonic acid (H₂CO₃), and phosphoric acid (H₃PO₄). In some embodiments, the acid is citric acid, acetic acid, or phosphoric acid. In some embodiments, the acid is citric acid. In some embodiments, the acid is acetic acid. In some embodiments, the acid is phosphoric acid.

In some embodiments, the acid is citric acid and citric acid is present in an amount of from about 0.005% to about 0.5% by weight. In some embodiments, citric acid is present in an amount of from about 0.01% to about 0.1% by weight. In some embodiments, citric acid is present in an amount of about 0.05% by weight.

In some embodiments, the acid is acetic acid and acetic acid is present in an amount of from about 0.005% to about 1% by weight. In some embodiments, acetic acid is present in an amount of from about 0.01% to about 0.5% by weight. In some embodiments, acetic acid is present in an amount of from about 0.05% to about 0.3% by weight. In some embodiments, acetic acid is present in an amount of about 0.05% by weight.

In some embodiments, the acid is phosphoric acid and phosphoric acid is present in an amount of from about 0.001% to about 0.03% by weight. In some embodiments, phosphoric acid is present in an amount of from about 0.001% to about 0.01% by weight. In some embodiments, phosphoric acid is present in an amount of about 0.005% by weight.

With reference to the composition according to the ninth aspect, in some embodiments, the composition includes DMSO in an amount of from about 0.1% to about 30% by weight, thereby reducing the hydrolysis of an ester, a lactone, a carbonate, a thiocarbonate, or a carbamate. In some embodiments, the composition includes DMSO in an amount of from about 0.1% to about 30% by weight, thereby reducing the hydrolysis of an ester or a carbonate. In some embodiments, the composition includes DMSO in an amount of from about 0.1% to about 30% by weight, thereby reducing the hydrolysis of an ester. In some embodiments, the composition includes DMSO in an amount of from about 0.1% to about 30% by weight, thereby reducing the hydrolysis of an aryl ester. In some embodiments, the composition includes DMSO in an amount of from about 0.1% to about 30% by weight, thereby reducing the hydrolysis of an aryl acetate.

With reference to the composition according to the ninth aspect, in some embodiments, DMSO is present in an amount of from about 0.1% to about 20% by weight. In some embodiments, DMSO is present in an amount of from about 1% to about 15% by weight of the base formulation, as defined and described herein (see Section IIIB-2. DMSO under Section III. Formulations).

With reference to the composition according to the ninth aspect, the ratio of DMSO to the combined one or more excipients is described herein (see Section IIIB-2. DMSO under Section III. Formulations). In some embodiments, one or more excipients are oleyl alcohol; and the ratio of DMSO to oleyl alcohol is from about 1:1 to about 5:1 or from about 1:1 to about 3:1 by weight. In some embodiments, the ratio of DMSO to oleyl alcohol is from about 1:1 to about 5:1 by weight. In some embodiments, the ratio of DMSO to oleyl alcohol is from about 1:1 to about 3:1 by weight.

In some embodiments of any one of compositions as described herein, the compound is represented by formula (V):

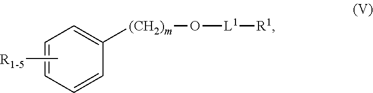

a hydrate, a solvate, a pharmaceutically acceptable salt, or a combination thereof;
wherein:
subscript m is an integer from 0 to 2;
L¹ is —C(O)—, —C(O)O—, —C(O)S—, or —C(O)NH—;
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl; and
each R represents a remainder of the compound.

In some embodiments, subscript m is 0 or 1. In some embodiments, subscript m is 1. In some embodiments, subscript m is 0.

In some embodiments, L¹ is —C(O)—. In some embodiments, L¹ is —C(O)O—. In some embodiments, L¹ is —C(O)S—. In some embodiments, L¹ is —C(O)NH—.

In some embodiments, subscript m is 0 and L¹ is —C(O)—.

In some embodiments, the compound of formula (V) is represented by formula (Va):

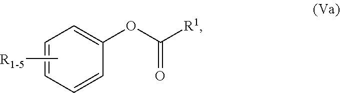

wherein R and R¹ are as defined and described herein.

In some embodiments, the compound of formula (V) is represented by formula (Va-1):

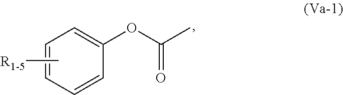

wherein R is as defined and described herein.

Each R group in any one of formula (V), (Va), and (Va-1) can be any functional group. Suitable functional groups include, but are not limited to, hydrogen, halogen, hydroxy, amino, amido, carboxy, sulfonate, sulfonyl, sulfonamide, alkyl, haloalkyl, cycloalkyl, alkenyl, alkynyl, alkoxy, heloalkoxy, heteroalkyl, heterocycloalkyl, aryl, and heteroaryl, each of which is unsubstituted or substituted with one or more functional groups.

In some embodiments of any one of formulae (V), (Va), and (Va-1), the compound has 1, 2, 3, 4, or 5 R groups, provided that at least one R is other than hydrogen. In some embodiments, the compound has one R group, provided that R is other than hydrogen. In some embodiments, the compound has two R groups, provided that at least one R is other than hydrogen. In some embodiments, the compound has three R groups, provided that at least one R is other than hydrogen. In some embodiments, the compound has four R groups, provided that at least one R is other than hydrogen. In some embodiments, the compound has five R groups, provided that at least one R is other than hydrogen.

In some embodiments, the compound of formula (V) is represented by formula (Vb):

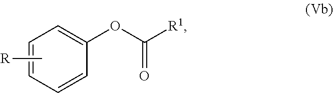

wherein R and R¹ are as defined and described herein.

In some embodiments, the compound of formula (V) is represented by formula (Vb-1):

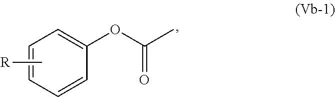

wherein R is as defined and described herein.

In some embodiments of any one of compositions as described herein, the compound has a moiety represented by the formula:

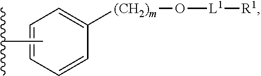

wherein:
subscript m is an integer from 0 to 2; and
L¹ is —C(O)—, —C(O)O—, —C(O)S—, or —C(O)NH—; and
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl.

In some embodiments of the moiety, L¹ is —C(O)—. In some embodiments, L¹ is —C(O)O—. In some embodiments, L¹ is —C(O)S—. In some embodiments, L¹ is —C(O)NH—.

In some embodiments of the moiety, subscript m is 0 and L¹ is —C(O)—.

In some embodiments of any one of compositions as described herein, the compound has a moiety represented by the formula:

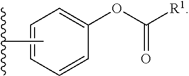

In some embodiments of any one of compositions as described herein, the compound has a moiety represented by the formula:

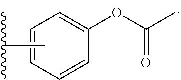

In some embodiments of any one of compositions as described herein, the compound is a compound having formula (I):

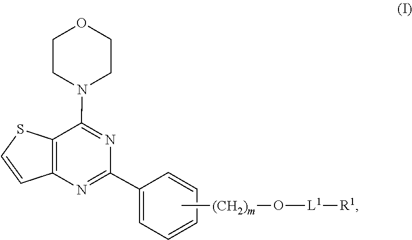

wherein subscript m, L¹, and R¹ are as defined and described herein.

In some embodiments, the compound of formula (I) in the composition is represented by formula (IIa):

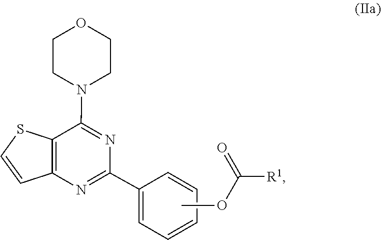

wherein R¹ is as defined and described herein.

In some embodiments, the compound of formula (I) in the composition is represented by any one of formulae (IIb), (IIc), and (IId):

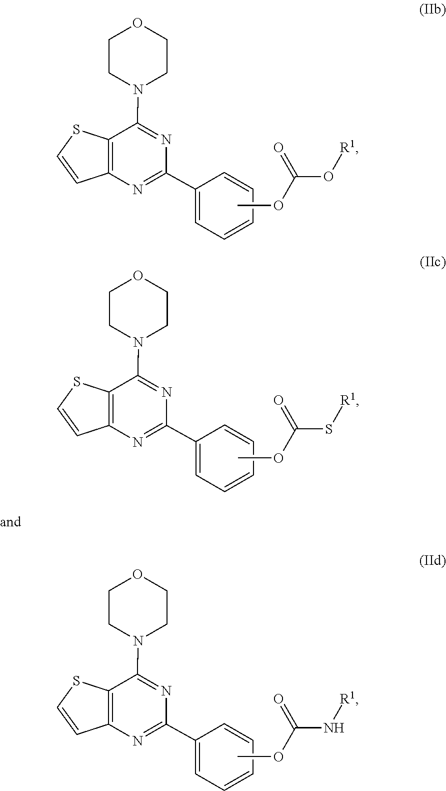

wherein R¹ is as defined herein in any aspect or embodiments described herein.

In some embodiments, the compound of formula (I) in the composition is represented by formula (IIa-1a):

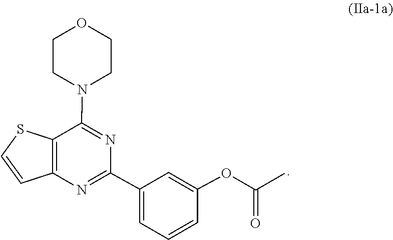 [SMILES: CC(=O)Oc1cccc(-c2nc(N3CCOCC3)c3sccc3n2)c1]

With reference to any one of compositions as described herein, the one or more excipients can be an unsaturated fatty alcohol, an unsaturated fatty acid, an unsaturated fatty ester, an unsaturated fatty ether, or a combination thereof, each of which is defined and described herein (see Section IIIA-1 or Section IIIB-1 under Section III. Formulations).

In some embodiments, one or more excipients include an unsaturated fatty alcohol as defined and described herein. In some embodiments, one or more excipients are an unsaturated fatty alcohol as defined and described herein. In some embodiments, the unsaturated fatty alcohol includes oleyl alcohol. In some embodiments, the unsaturated fatty alcohol is oleyl alcohol. In some embodiments, one or more excipients include oleyl alcohol. In some embodiments, one or more excipients are oleyl alcohol.

In some embodiments of any one of compositions as described herein, the one or more additional excipients are present. The one or more additional excipients can be one or more solvents as defined and described herein (see Section IIIA-4 when an acid is present and Section IIIB-3 when DMSO is present under Section III. Formulations).

In some embodiments, the composition is a topical formulation.

In some embodiments, the composition is a formulation including the compound of formula (I), each of the formulation and the compound of formula (I) is defined and described herein according to Sections IIIA, IIIC, IIID, and IIIE under Section III. Formulations. In some embodiments, the composition is any one of formulations (AA), (AA1ca), (AA2ca), (AA1aa), (AA2aa), (AA1pa), (AA2pa), (AB), (AB1ca), (AB1aa), (AB1pa), (AC), (AC1ca), (AC1aa), and (AC1pa), as defined and described herein (see Section IIIA-9. Selected Embodiments under Section III. Formulations).

In some embodiments, the composition is the formulation including the compound of formula (I), each of the formulation and the compound of formula (I) is defined and described herein according to Sections IIIB, IIIC, IIID, and IIIE under Section III. Formulations. In some embodiments, the composition is any one of formulations (BA), (BA-1), (BA1), (BA1-1), (BA2), (BA2-1), (BB), (BB-1), (BB1), and (BB1-1) to (BB1-6), as defined and described herein (see Section IIIB-7. Selected Embodiments under Section III. Formulations).

With reference to the composition according to the eighth aspect, the composition can be prepared by methods including, for example simply mixing components of a) to e). Alternatively, the composition can be prepared by sonicating a mixture of a) to e). In some embodiments, the composition is prepared by mixing components of a) to e). In some embodiments, the composition is prepared by sonicating a mixture of a) to e).

With reference to the composition according to the ninth aspect, the composition can be prepared by methods including, for example sonicating a first mixture of a) to b) followed by mixing with components of c) and d). In some embodiments, the composition is prepared by 1) mixing components of a) and b) to form a first mixture; 2) sonicating the first mixture to form a second mixture; and mixing the second mixture with components of c) and d).

### VIII. Embodiments

### A. Embodiments for Formulations Including an Acid

Embodiment A1: A topical formulation, comprising:
a) a compound having formula (I):

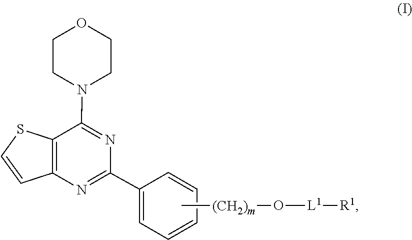

a hydrate, a solvate, a pharmaceutically acceptable salt, or a combination thereof;
wherein:
   subscript m is an integer from 0 to 2; and
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) DMSO in an amount of from about 0% to 20% by weight of the base formulation;
c) one or more excipients selected from the group consisting of an unsaturated fatty alcohol, an unsaturated fatty acid, an unsaturated fatty ester, and an unsaturated fatty ether;
d) one or more solvents; and
e) an acid in an amount of no more than 1% by weight of the base formulation,
   wherein
the one or more excipients are in an amount of no more than 10% by weight of the base formulation;
the one or more solvents comprise a C₂₋₆ alkylene glycol, a di-(C₂₋₆ alkylene) glycol, or a combination thereof; and
a hydrolysis of the compound having formula (I) to a corresponding compound having formula (IV):

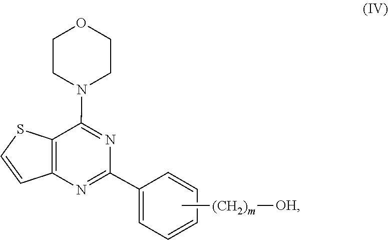

is less than 10% over a period of 10 days at a temperature of 80° C. (±2° C.) or over a period of 6 months at a temperature of 40° C. (±2° C.) and a relative humidity of 75% (±5%), wherein subscript m is an integer from 0 to 2.

Embodiment A2: The topical formulation of embodiment A1, wherein the one or more excipients are oleyl alcohol.

Embodiment A3: The topical formulation of embodiment A2, wherein oleyl alcohol is present in an amount of from 1% to 10%, from 2% to 7%, from 3% to 7%, from 3% to 6%, or from 3% to 5% by weight of the base formulation.

Embodiment A4: The topical formulation of embodiment A3, wherein oleyl alcohol is present in an amount of about 5% by weight of the base formulation.

Embodiment A5: The topical formulation of any one of embodiments A1 to A4, wherein DMSO, when present, is in an amount of from 5% to 20%, from 5% to 15%, from 10% to 15%, or from 5% to 10% by weight of the base formulation.

Embodiment A6: The topical formulation of embodiment A5, wherein DMSO, when present, is in an amount of about 5% or about 15% by weight of the base formulation.

Embodiment A7: The topical formulation of any one of embodiments A1 to A4, wherein DMSO is absent.

Embodiment A8: The topical formulation of any one of embodiments A1 to A7, wherein the acid is an organic acid having a pKa of from about 3 to about 6.

Embodiment A9: The topical formulation of any one of embodiments A1 to A7, wherein the acid is an inorganic acid selected from the group consisting of hydrochloric acid (HCl), boric acid (H₃BO₃), sulfuric acid (H₂SO₄), carbonic acid (H₂CO₃), and phosphoric acid (H₃PO₄).

Embodiment A10: The topical formulation of any one of embodiments A1 to A7, wherein the acid is citric acid, acetic acid, or phosphoric acid.

Embodiment A11: The topical formulation of embodiment A10, wherein the acid is citric acid present in an amount of from 0.005% to 0.5% by weight of the base formulation.

Embodiment A12: The topical formulation of embodiment A11, wherein citric acid is present in an amount of from 0.01% to 0.1% by weight of the base formulation.

Embodiment A13: The topical formulation of embodiment A12, wherein citric acid is present in an amount of about 0.05% by weight of the base formulation.

Embodiment A14: The topical formulation of embodiment A10, wherein the acid is acetic acid present in an amount of from 0.005% to 1% by weight of the base formulation.

Embodiment A15: The topical formulation of embodiment A14, wherein acetic acid is present in an amount of from 0.01% to 0.5% by weight of the base formulation.

Embodiment A16: The topical formulation of embodiment A15, wherein acetic acid is present in an amount of about 0.05% by weight of the base formulation.

Embodiment A17: The topical formulation of embodiment A10, wherein the acid is phosphoric acid present in an amount of from 0.001% to 0.03% by weight of the base formulation.

Embodiment A18: The topical formulation of embodiment A17, wherein phosphoric acid is present in an amount of from 0.001% to 0.01% by weight of the base formulation.

Embodiment A19: The topical formulation of embodiment A18, wherein phosphoric acid is present in an amount of about 0.005% by weight of the base formulation.

Embodiment A20: The topical formulation of any one of embodiments A1 to A19, wherein the one or more solvents further comprise C₁₋₃ alkyl-(OCH₂CH₂)₁₋₅—OH, a polyethylene glycol, or a combination thereof.

Embodiment A21: The topical formulation of embodiment A20, wherein C₁₋₃ alkyl-(OCH₂CH₂)₁₋₅—OH is 2-(2-ethoxyethoxy)ethanol; the C₂₋₆ alkylene glycol is propylene glycol; the di-(C₂₋₆ alkylene) glycol is dipropylene glycol; and the polyethylene glycol is PEG400.

Embodiment A22: The topical formulation of any one of embodiments A1 to A21, wherein the one or more solvents comprise 2-(2-ethoxyethoxy)ethanol, propylene glycol, and dipropylene glycol.

Embodiment A23: The topical formulation of any one of embodiments A1 to A21, wherein the one or more solvents comprise 2-(2-ethoxyethoxy)ethanol, propylene glycol, dipropylene glycol, and PEG400.

Embodiment A24: The topical formulation of embodiment A22 or A23, wherein 2-(2-ethoxyethoxy)ethanol is present in an amount of from 20% to 40%, from 20% to 35%, or from 20% to 30% by weight of the base formulation.

Embodiment A25: The topical formulation of embodiment A24, wherein 2-(2-ethoxyethoxy)ethanol is present in an amount of about 25% by weight of the base formulation.

Embodiment A26: The topical formulation of any one of embodiments A22 to A25, wherein propylene glycol is present in an amount of from 30% to 70%, from 30% to 60%, from 40% to 60%, or from 45% to 55% by weight of the base formulation.

Embodiment A27: The topical formulation of embodiment A26, wherein propylene glycol is present in an amount of about 50% by weight of the base formulation.

Embodiment A28: The topical formulation of embodiment A26, wherein propylene glycol is present in an amount of about 45% by weight of the base formulation.

Embodiment A29: The topical formulation of any one of embodiments A22 to A28, wherein dipropylene glycol is present in an amount of from 1% to 25%, from 1% to 20%, or from 1% to 10% by weight of the base formulation.

Embodiment A30: The topical formulation of embodiment A29, wherein dipropylene glycol is present in an amount of from 1% to 10% by weight of the base formulation.

Embodiment A31: The topical formulation of embodiment A30, wherein dipropylene glycol is present in an amount of about 5% by weight of the base formulation.

Embodiment A32: The topical formulation of any one of embodiments A23 to A31, wherein PEG400 is present in an amount of from 5% to 50%, from 5% to 40%, from 5% to 30%, from 5% to 20%, or from 5% to 15% by weight of the base formulation.

Embodiment A33: The topical formulation of embodiment A32, wherein, when DMSO is present, PEG400 is present in an amount of from 5% to 15% by weight of the base formulation.

Embodiment A34: The topical formulation of embodiment A33, wherein PEG400 is present in an amount of about 10% by weight of the base formulation.

Embodiment A35: The topical formulation of embodiment A32, wherein, when DMSO is absent, PEG400 is present in an amount of from 15% to 30% by weight of the base formulation.

Embodiment A36: The topical formulation of embodiment A35, wherein PEG400 is present in an amount of about 22% by weight of the base formulation.

Embodiment A37: The topical formulation of any one of embodiments A1 to A36, further comprising a gelling agent.

Embodiment A38: The topical formulation of embodiment A37, wherein the gelling agent is hydroxypropyl cellulose in an amount of 1% to 5%, from 1% to 4%, or from 1% to 3% by weight of the base formulation.

Embodiment A39: The topical formulation of embodiment A38, wherein hydroxypropyl cellulose is present in an amount of about 2% by weight of the base formulation.

Embodiment A40: The topical formulation of embodiment A1, comprising:
a) the compound of formula (I);
b) DMSO in an amount of about 5% by weight of the base formulation;
c) oleyl alcohol in an amount of about 5% by weight of the base formulation;
d) 2-(2-ethoxyethoxy)ethanol, propylene glycol, dipropylene glycol, and PEG400, which are present in an amount of about 25%, about 50%, about 5%, and about 10%, respectively, by weight of the base formulation;
e) citric acid in an amount of about 0.05% by weight of the base formulation; and
f) hydroxypropyl cellulose in an amount of about 2% by weight of the base formulation.

Embodiment A41: The topical formulation of embodiment A1, comprising:
a) the compound of formula (I);
b) DMSO in an amount of about 5% by weight of the base formulation;
c) oleyl alcohol in an amount of about 3% by weight of the base formulation;
d) 2-(2-ethoxyethoxy)ethanol, propylene glycol, dipropylene glycol, and PEG400, which are present in an amount of about 25%, about 52%, about 5%, and about 10%, respectively, by weight of the base formulation;
e) citric acid in an amount of about 0.05% by weight of the base formulation; and
f) hydroxypropyl cellulose in an amount of about 2% by weight of the base formulation.

Embodiment A42: The topical formulation of embodiment A1, comprising:
a) the compound of formula (I);
b) DMSO in an amount of about 15% by weight of the base formulation;
c) oleyl alcohol in an amount of about 5% by weight of the base formulation;
d) 2-(2-ethoxyethoxy)ethanol, propylene glycol, and dipropylene glycol, which are present in an amount of about 25%, about 50%, and about 5%, respectively, by weight of the base formulation;
e) citric acid in an amount of about 0.05% by weight of the base formulation; and
f) hydroxypropyl cellulose in an amount of about 2% by weight of the base formulation.

Embodiment A43: The topical formulation of embodiment A1, comprising:
a) the compound of formula (I);
c) oleyl alcohol in an amount of about 4.5% by weight of the base formulation;
d) 2-(2-ethoxyethoxy)ethanol, propylene glycol, dipropylene glycol, and PEG400, which are present in an amount of about 23%, about 45.5%, about 4.5%, and about 22.5%, respectively, by weight of the base formulation;
e) citric acid in an amount of about 0.05% by weight of the base formulation; and
f) hydroxypropyl cellulose in an amount of about 2% by weight of the base formulation,
   wherein the topical formulation is free of DMSO.

Embodiment A44: The topical formulation of any one of embodiments A1 to A43, wherein compound of formula (I) is present in a degree to saturation of from 75% to 100%.

Embodiment A45: The topical formulation of embodiment A44, wherein the compound of formula (I) is present in a degree to saturation of from 90% to 100%.

Embodiment A46: The topical formulation of any one of embodiments A1 to A43, wherein the compound of formula (I) is present in an amount of 0.05% to 15%, from 0.5% to 10%, from 1% to 10%, from 2% to 10%, from 5% to 10%, or from 2 to 5% by weight of the base formulation on a salt-free and anhydrous basis.

Embodiment A47: The topical formulation of any one of embodiments A1 to A46, wherein the topical formulation has an apparent pH value of from about 4 to about 5.

Embodiment A48: The topical formulation of any one of embodiments A1 to A47, wherein a skin flux of the compound having formula (I) has an increase of greater than 2 fold as compared to a skin flux of the corresponding compound having formula (IV) in the same topical formulation, provided that the compound having formula (I) and the compound having formula (IV) have the same degree to saturation.

Embodiment A49: The topical formulation of any one of embodiments A1 to A48, wherein the compound of formula (I) is represented by formula (IIa):

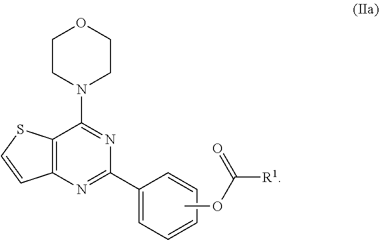

Embodiment A50: The topical formulation of any one of embodiments A1 to A49, wherein the compound of formula (I) is represented by formula (IIa-1a):

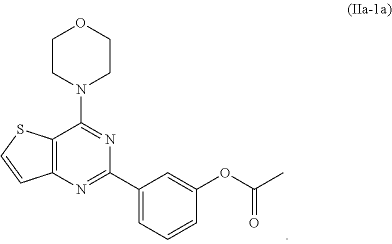 [SMILES: CC(=O)Oc1cccc(-c2nc(N3CCOCC3)c3sccc3n2)c1]

Embodiment A51: The topical formulation of embodiment A50, wherein a cumulative skin flux of the compound having formula (IIa-1a) is at least 1 μg/cm²/hour, 2 μg/cm²/hour, or 3 μg/cm²/hour at 24 hours, as measured by a Franz diffusion cell using a human cadaver skin.

Embodiment A52: The topical formulation of embodiment A50 or A51, wherein a cumulative skin flux of the compound having formula (IIa-1a) is at least 3 μg/cm²/hour at 24 hours, as measured by a Franz diffusion cell using a human cadaver skin.

Embodiment A53: The topical formulation of any one of embodiments A1 to A52, wherein the topical formulation is a foam, a lotion, a pray, an aerosol, an ointment, a cream, a gel, a paste, a patch, or an in-situ patch.

Embodiment A54: A process for preparing a topical formulation according to embodiment A1, comprising:
1) forming a first mixture comprising:
   a) a compound having formula (I):

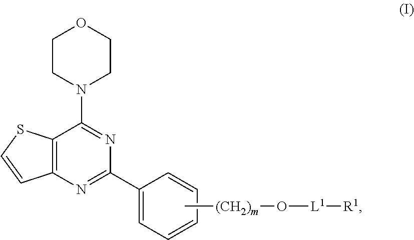

a hydrate, a solvate, a pharmaceutically acceptable salt, or a combination thereof;
wherein:
   subscript m is an integer from 0 to 2; and
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) DMSO in an amount of from 0% to 20% by weight of the base formulation;
c) one or more excipients selected from the group consisting of an unsaturated fatty alcohol, an unsaturated fatty acid, an unsaturated fatty ester, and an unsaturated fatty ether;
d) one or more solvents; and
e) an acid in an amount of no more than 1% by weight of the base formulation,
2) mixing the first mixture to form a uniform mixture,
   wherein
the one or more excipients are in an amount of no more than 10% by weight of the base formulation; and
the one or more solvents comprise a C₂₋₆ alkylene glycol, a di-(C₂₋₆ alkylene) glycol, or a combination thereof.

Embodiment A55: The process of embodiment A54, further comprising, prior to step 1), heating any one or more of b), c), and e) at a temperature of from 40° C. to 50° C. for a period of from about 30 to about 120 minutes.

Embodiment A56: The process of embodiment A54, further comprising, prior to step 1), forming a premixture of b) and c); and mixing the premixture for a period of from about 30 to about 120 minutes at room temperature.

Embodiment A57: The process of any one of embodiments A54 to A56, further comprising:
3) adding a gelling agent to the uniform mixture of step 2) to form a second mixture; and
4) blending the second mixture to form a topical formulation according to embodiment A37.

Embodiment A58: The process of any one of embodiments A54 to A57, wherein the compound of formula (I) is represented by formula (IIa-1a):

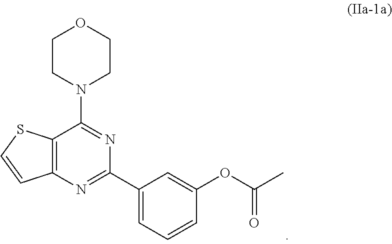 [SMILES: CC(=O)Oc1cccc(-c2nc(N3CCOCC3)c3sccc3n2)c1]

Embodiment A59: A topical formulation, prepared by a process according to any one of embodiments A54 to A58.

Embodiment A60: A method of treating a vascular malformation in a subject in need thereof, comprising administering to the subject an effective amount of the topical formulation of any one of embodiments A1 to A53 and A59.

Embodiment A61: The method of embodiment A60, wherein the vascular malformation is a venous malformation, an arterial malformation, an arteriovenous malformation, or a lymphatic malformation.

Embodiment A62: The method of embodiment A60 or A61, wherein the topical formulation comprises a compound represented by formula (IIa-1a):

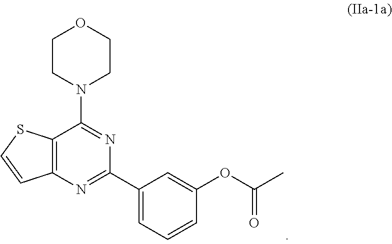 [SMILES: CC(=O)Oc1cccc(-c2nc(N3CCOCC3)c3sccc3n2)c1]

Embodiment A63: The method of any one of embodiments A60 to A62, wherein the topical formulation is administered as a foam, a lotion, a spray, an aerosol, an ointment, a cream, a gel, a paste, a patch, or an in-situ patch.

Embodiment A64: A stable composition, comprising:
a) a compound comprising one or more groups selected from the group consisting of an ester, a lactone, an amide, a lactam, a carbonate, a thiocarbonate, and a carbamate, provided that the amide is other than C(O)NH₂;
b) DMSO in an amount of from 0% to 30% by weight;
c) one or more excipients selected from the group consisting of an unsaturated fatty alcohol, an unsaturated fatty acid, an unsaturated fatty ester, and an unsaturated fatty ether; and
d) optionally one or more solvents; and
e) an acid in an amount of no more than 1% by weight,
   wherein
the one or more excipients are in an amount of no more than 10% by weight; and
the compound maintains a relative purity of at least 90% over a period of 10 days at a temperature of 80° C. (±2° C.) or over a period of 6 months at a temperature of 40° C. (±2° C.) and a relative humidity of 75% (±5%).

Embodiment A65: The stable composition of embodiment A64, wherein the compound is represented by formula (V):

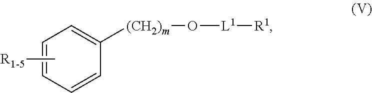

a hydrate, a solvate, a pharmaceutically acceptable salt, or a combination thereof;
wherein:
subscript m is an integer from 0 to 2; and
L¹ is —C(O)—, —C(O)O—, —C(O)S—, or —C(O)NH—;
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl; and
each R independently represents a remainder of the compound.

Embodiment A66: The stable composition of embodiment A64 or A65, wherein the compound of formula (V) is represented by formula (Va):

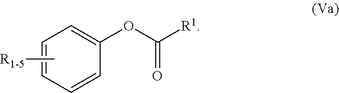

Embodiment A67: The stable composition of any one of embodiments A64 to A66, wherein the compound of formula (V) is represented by formula (Va-1):

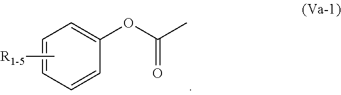

Embodiment A68: The stable composition of embodiment A64, wherein the compound has a moiety represented by the formula:

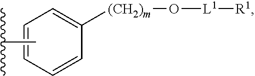

wherein:
subscript m is an integer from 0 to 2; and
L¹ is —C(O)—, —C(O)O—, —C(O)S—, or —C(O)NH—;
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl.

Embodiment A69: The stable composition of embodiment A68, wherein the moiety is represented by formula:

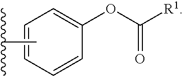

Embodiment A70: The stable composition of embodiment A68 or A69, wherein the moiety is represented by formula:

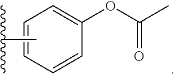

Embodiment A71: The stable composition of embodiment A64, wherein the compound is represented by formula (IIa-1a):

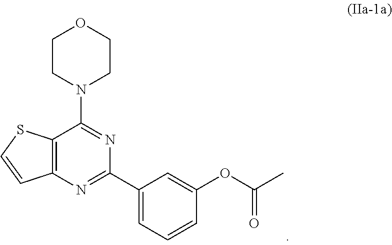 [SMILES: CC(=O)Oc1cccc(-c2nc(N3CCOCC3)c3sccc3n2)c1]

Embodiment A72: The stable formulation of any one of embodiments A64 to A71, wherein the one or more excipients are oleyl alcohol.

Embodiment A73: The stable formulation of any one of embodiments A64 to A72, wherein DMSO is present in an amount of from 1% to 15% by weight.

Embodiment A74: The stable formulation of any one of embodiments A64 to A72, wherein DMSO is absent.

Embodiment A75: The stable formulation of any one of embodiments A64 to A74, wherein the acid is an organic acid having a pKa of from about 3 to about 6.

Embodiment A76: The stable formulation of any one of embodiments A64 to A74, wherein the acid is an inorganic acid selected from the group consisting of hydrochloric acid (HCl), boric acid (H₃BO₃), sulfuric acid (H₂SO₄), carbonic acid (H₂CO₃), and phosphoric acid (H₃PO₄).

Embodiment A77: The stable formulation of any one of embodiments A64 to A74, wherein the acid is citric acid, acetic acid, or phosphoric acid.

Embodiment A78: The stable formulation of embodiment A77, wherein the acid is citric acid present in an amount of from 0.005% to 0.5% by weight.

Embodiment A79: The stable formulation of embodiment A78, wherein citric acid is present in an amount of from 0.01% to 0.1% by weight.

Embodiment A80: The stable formulation of embodiment A79, wherein citric acid is present in an amount of about 0.05% by weight.

Embodiment A81: The stable formulation of embodiment A11, wherein the acid is acetic acid present in an amount of from 0.005% to 1% by weight.

Embodiment A82: The stable formulation of embodiment A81, wherein acetic acid is present in an amount of from 0.01% to 0.5% by weight.

Embodiment A83: The stable formulation of embodiment A82, wherein acetic acid is present in an amount of about 0.05% by weight.

Embodiment A84: The stable formulation of embodiment A77, wherein the acid is phosphoric acid present in an amount of from 0.001% to 0.03% by weight.

Embodiment A85: The stable formulation of embodiment A84, wherein phosphoric acid is present in an amount of from 0.001% to 0.01% by weight.

Embodiment A86: The stable formulation of embodiment A85, wherein phosphoric acid is present in an amount of about 0.005% by weight.

Embodiment A87: The stable formulation of any one of embodiments A64 to A86, is a topical formulation.

### B. Embodiments for Formulations Including DMSO

Embodiment B1: A topical formulation, comprising:
a) a compound having formula (I):

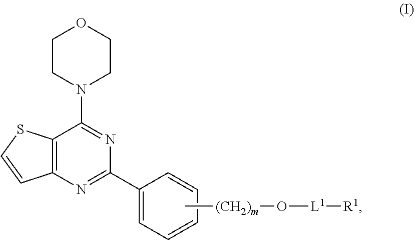

a hydrate, a solvate, a pharmaceutically acceptable salt, or a combination thereof;
wherein:
   subscript m is an integer from 0 to 2; and
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) DMSO in an amount of from 1% to 20% by weight of the base formulation;
c) one or more excipients selected from the group consisting of an unsaturated fatty alcohol, an unsaturated fatty acid, an unsaturated fatty ester, and an unsaturated fatty ether; and
d) one or more solvents,
   wherein
the one or more excipients are in an amount of no more than 10% by weight of the base formulation;
the ratio of DMSO to the combined one or more excipients is at least 1:1 by weight; the one or more solvents comprise a C₂₋₆ alkylene glycol, a di-(C₂₋₆ alkylene) glycol, or a combination thereof; and
hydrolysis of the compound having formula (I) to a corresponding compound having formula (IV):

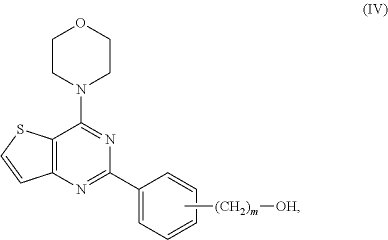

is less than 10% over a period of 10 days at a temperature of 80° C. (±2° C.) or over a period of 6 months at a temperature of 40° C. (±2° C.) and a relative humidity of 75% (±5%), wherein subscript m is an integer from 0 to 2.

Embodiment B2: The topical formulation of embodiment B1, prepared by sonicating a mixture of a) to d).

Embodiment B3: The topical formulation of embodiment B1 or B2, wherein the one or more excipients are oleyl alcohol.

Embodiment B4: The topical formulation of embodiment B3, wherein oleyl alcohol is present in an amount of from 1% to 10%, from 2% to 7%, from 3% to 7%, from 3% to 6%, or from 3% to 5% by weight of the base formulation.

Embodiment B5: The topical formulation of embodiment B4, wherein oleyl alcohol is present in an amount of about 5% by weight of the base formulation.

Embodiment B6: The topical formulation of any one of embodiments B1 to B5, wherein DMSO is present in an amount of from 5% to 20%, from 5% to 15%, from 10% to 15%, or from 5% to 10% by weight of the base formulation.

Embodiment B7: The topical formulation of embodiment B6, wherein DMSO is present in an amount of about 5% or about 15% by weight of the base formulation.

Embodiment B8: The topical formulation of any one of embodiments B1 to B7, wherein the ratio of DMSO to oleyl alcohol is from 1:1 to 5:1 by weight.

Embodiment B9: The topical formulation of embodiment B8, wherein the ratio of DMSO to oleyl alcohol is from 1:1 to 3:1 by weight.

Embodiment B10: The topical formulation of any one of embodiments B1 to B9, wherein the one or more solvents further comprise C₁₋₃ alkyl-(OCH₂CH₂)₁₋₅—OH, a polyethylene glycol, or a combination thereof.

Embodiment B11: The topical formulation of embodiment B10, wherein C₁₋₃ alkyl-(OCH₂CH₂)₁₋₅—OH is 2-(2-ethoxyethoxy)ethanol; the C₂₋₆ alkylene glycol is propylene glycol; the di-(C₂₋₆ alkylene) glycol is dipropylene glycol; and the polyethylene glycol is PEG400.

Embodiment B12: The topical formulation of any one of embodiments B1 to B11, wherein the one or more solvents comprise 2-(2-ethoxyethoxy)ethanol, propylene glycol, and dipropylene glycol.

Embodiment B13: The topical formulation of any one of embodiments B1 to B11, wherein the one or more solvents comprise 2-(2-ethoxyethoxy)ethanol, propylene glycol, dipropylene glycol, and PEG400.

Embodiment B14: The topical formulation of embodiment B12 or B13, wherein 2-(2-ethoxyethoxy)ethanol is present in an amount of from 20% to 40%, from 20% to 35%, or from 20% to 30% by weight of the base formulation.

Embodiment B15: The topical formulation of embodiment B14, wherein 2-(2-ethoxyethoxy)ethanol is present in an amount of about 25% by weight of the base formulation.

Embodiment B16: The topical formulation of any one of embodiments B12 to B15, wherein propylene glycol is present in an amount of from 30% to 70%, from 30% to 60%, from 40% to 60%, or from 45% to 55% by weight of the base formulation.

Embodiment B17: The topical formulation of embodiment B16, wherein propylene glycol is present in an amount of about 50% by weight of the base formulation.

Embodiment B18. The topical formulation of any one of embodiments B12 to B17, wherein dipropylene glycol is present in an amount of from 1% to 25%, from 1% to 20%, or from 1% to 10% by weight of the base formulation.

Embodiment B19. The topical formulation of embodiment B18, wherein dipropylene glycol is present in an amount of from 1% to 10% by weight of the base formulation.

Embodiment B20. The topical formulation of embodiment B19, wherein dipropylene glycol is present in an amount of about 5% by weight of the base formulation.

Embodiment B21. The topical formulation of any one of embodiments B13 to B20, wherein PEG400 is present in an amount of from 5% to 50%, from 5% to 40%, from 5% to 30%, from 5% to 20%, or from 5% to 15% by weight of the base formulation.

Embodiment B22: The topical formulation of embodiment B21, wherein PEG400 is present in an amount of from 5% to 15% by weight of the base formulation.

Embodiment B23: The topical formulation of embodiment B22, wherein PEG400 is present in an amount of about 10% by weight of the base formulation.

Embodiment B24: The topical formulation of any one of embodiments B1 to B23, further comprising a gelling agent.

Embodiment B25: The topical formulation of embodiment B24, wherein the gelling agent is hydroxypropyl cellulose in an amount of 1% to 5%, from 1% to 4%, or from 1% to 3% by weight of the base formulation.

Embodiment B26: The topical formulation of embodiment B25, wherein hydroxypropyl cellulose is present in an amount of about 2% by weight of the base formulation.

Embodiment B27: The topical formulation of embodiment B1 or B2, comprising:
a) the compound of formula (I);
b) DMSO in an amount of about 5% by weight of the base formulation;
c) oleyl alcohol in an amount of about 5% by weight of the base formulation;
d) 2-(2-ethoxyethoxy)ethanol, propylene glycol, dipropylene glycol, and PEG400, which are present in an amount of about 25%, about 50%, about 5%, and about 10%, respectively, by weight of the base formulation; and
e) hydroxypropyl cellulose in an amount of about 2% by weight of the base formulation.

Embodiment B28: The topical formulation of embodiment B1 or B2, comprising:
a) the compound of formula (I);
b) DMSO in an amount of about 5% by weight of the base formulation;
c) oleyl alcohol in an amount of about 3% by weight of the base formulation;
d) 2-(2-ethoxyethoxy)ethanol, propylene glycol, dipropylene glycol, and PEG400, which are present in an amount of about 25%, about 52%, about 5%, and about 10%, respectively, by weight of the base formulation; and
e) hydroxypropyl cellulose in an amount of about 2% by weight of the base formulation.

Embodiment B29: The topical formulation of embodiment B1 or B2, comprising:
a) the compound of formula (I);
b) DMSO in an amount of about 15% by weight of the base formulation;
c) oleyl alcohol in an amount of about 5% by weight of the base formulation;
d) 2-(2-ethoxyethoxy)ethanol, propylene glycol, and dipropylene glycol, which are present in an amount of about 25%, about 50%, and about 5%, respectively, by weight of the base formulation; and
e) hydroxypropyl cellulose in an amount of about 2% by weight of the base formulation.

Embodiment B30: The topical formulation of any one of embodiments B1 to B29, wherein the compound of formula (I) is present in a degree to saturation of from 75% to 100%.

Embodiment B31: The topical formulation of embodiment B30, wherein the compound of formula (I) is present in a degree to saturation of from 90% to 100%.

Embodiment B32: The topical formulation of any one of embodiments B1 to B29, wherein the compound of formula (I) is present in an amount of 0.05% to 15%, from 0.5% to 10%, from 1% to 10%, from 2% to 10%, from 5% to 10%, or from 2 to 5% by weight of the base formulation on a salt-free and anhydrous basis.

Embodiment B33: The topical formulation of any one of embodiments B1 to B32, wherein a skin flux of the compound having formula (I) has an increase of greater than 2 fold as compared to a skin flux of the corresponding compound having formula (IV) in the same topical formulation, provided that the compound having formula (I) and the compound having formula (IV) have the same degree to saturation.

Embodiment B34: The topical formulation of any one of embodiments B1 to B33, wherein the compound of formula (I) is represented by formula (IIa):

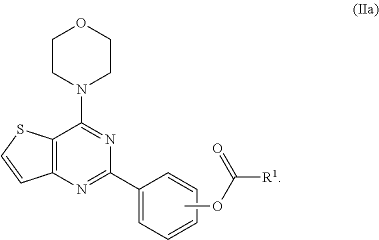

Embodiment B35: The topical formulation of any one of embodiments B1 to B34, wherein the compound of formula (I) is represented by formula (IIa-1a):

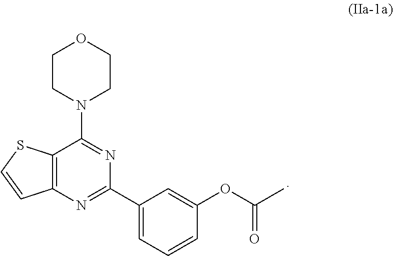 [SMILES: CC(=O)Oc1cccc(-c2nc(N3CCOCC3)c3sccc3n2)c1]

Embodiment B36: The topical formulation of embodiment B35, wherein the compound having formula (IIa-1a) has a cumulative skin flux of at least 1 μg/cm²/hour, 2 μg/cm²/hour, or 3 μg/cm²/hour at 24 hours, as measured by a Franz diffusion cell using a human cadaver skin.

Embodiment B37: The topical formulation of embodiment B35 or B36, wherein the compound having formula (IIa-1a) has a cumulative skin flux of at least 3 μg/cm²/hour at 24 hours, as measured by a Franz diffusion cell using a human cadaver skin.

Embodiment B38: The topical formulation of any one of embodiments B1 to B37, wherein the topical formulation is a foam, a lotion, a spray, an aerosol, an ointment, a cream, a gel, a paste, a patch, or an in-situ patch.

Embodiment B39: A process for preparing a topical formulation according to embodiment B1, comprising:
1) forming a first mixture comprising:
   a) a compound having formula (I):

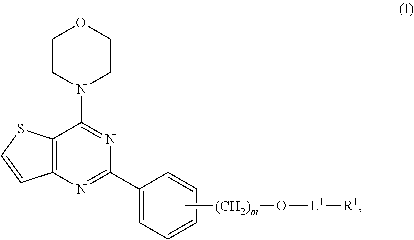

a hydrate, a solvate, a pharmaceutically acceptable salt, or a combination thereof;
wherein:
   subscript m is an integer from 0 to 2; and
   L¹ is —C(O)—, —C(O)O—, —C(O)S—, or —C(O)NH—; and
   R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) DMSO in an amount of from 1% to 20% by weight of the base formulation;
c) one or more excipients selected from the group consisting of an unsaturated fatty alcohol, an unsaturated fatty acid, an unsaturated fatty ester, and an unsaturated fatty ether; and
d) one or more solvents; and
2) sonicating the first mixture to form a second mixture,
   wherein
the one or more excipients are in an amount of no more than 10% by weight of the base formulation;
the ratio of DMSO to the combined one or more excipients is at least 1:1 by weight; and
the one or more solvents comprise a C₂₋₆ alkylene glycol, a di-(C₂₋₆ alkylene) glycol, or a combination thereof.

Embodiment B40: The process of embodiment B39, wherein the first mixture is sonicated for a period of from about 30 to about 60 minutes.

Embodiment B41: The process of embodiment B39 or B40, further comprising, prior to step 1), forming a premixture of b) and c) and sonicating the premixture of b) and c) for a period of from about 10 to about 60 minutes.

Embodiment B42: The process of embodiment B39 or B40, further comprising, prior to step 1), forming a premixture of b) and c) and heating the premixture of b) and c) for a period of from 1 to 10 days at a temperature of about 80° C.

Embodiment B43: The process of any one of embodiments B39 to B42, further comprising:
3) adding a gelling agent to the second mixture of step 2) to form a third mixture; and
4) blending the third mixture to form a topical formulation according to claim 24.

Embodiment B44: The process of any one of embodiments B39 to B43, wherein the compound of formula (I) is represented by formula (IIa-1a):

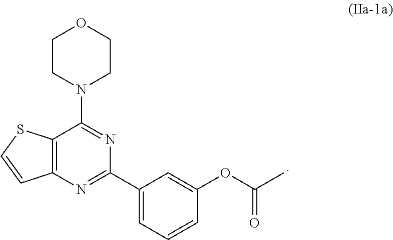 [SMILES: CC(=O)Oc1cccc(-c2nc(N3CCOCC3)c3sccc3n2)c1]

Embodiment B45: A topical formulation, prepared by a process according to any one of embodiments B39 to B44.

Embodiment B46: A method of treating a vascular malformation in a subject in need thereof, comprising administering to the subject an effective amount of the topical formulation of any one of embodiments B1 to B38 and B45.

Embodiment B47: The method of embodiment B46, wherein the vascular malformation is a venous malformation, an arterial malformation, an arteriovenous malformation, or a lymphatic malformation.

Embodiment B48: The method of embodiment B46 or B47, wherein the topical formulation comprises a compound represented by formula (IIa-1a):

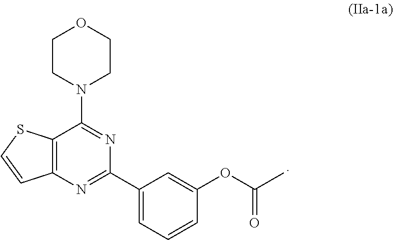 [SMILES: CC(=O)Oc1cccc(-c2nc(N3CCOCC3)c3sccc3n2)c1]

Embodiment B49: The method of any one of embodiments B46 to B48, wherein the topical formulation is administered as a foam, a lotion, a spray, an aerosol, an ointment, a cream, a gel, a paste, a patch, or an in-situ patch.

Embodiment B50: A stable composition, comprising:
a) a compound comprising one or more groups selected from the group consisting of an ester, a lactone, an amide, a lactam, a carbonate, a thiocarbonate, and a carbamate, provided that the amide is other than C(O)NH₂;
b) DMSO in an amount of from 0.1% to 30% by weight;
c) one or more excipients selected from the group consisting of an unsaturated fatty alcohol, an unsaturated fatty acid, an unsaturated fatty ester, and an unsaturated fatty ether; and
d) optionally one or more additional excipients,
   wherein
the one or more excipients are in an amount of no more than 10% by weight;
the ratio of DMSO to the combined one or more excipients is at least 1:1 by weight; and
the compound maintains a relative purity of at least 90% over a period of 10 days at a temperature of 80° C. (±2° C.) or over a period of 6 months at a temperature of 40° C. (±2° C.) and a relative humidity of 75% (±5%).

Embodiment B51: The stable composition of embodiment B50, wherein the compound is represented by formula (V):

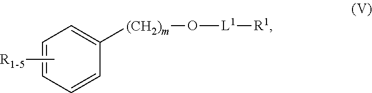

a hydrate, a solvate, a pharmaceutically acceptable salt, or a combination thereof;
wherein:
subscript m is an integer from 0 to 2; and
L¹ is —C(O)—, —C(O)O—, —C(O)S—, or —C(O)NH—;
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl; and
each R represents a remainder of the compound.

Embodiment B52: The stable composition of embodiment B50 or B51, wherein the compound of formula (V) is represented by formula (Va):

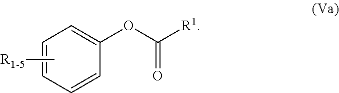

Embodiment B53: The stable composition of any one of embodiments B50 to B52, wherein the compound of formula (V) is represented by formula (Va-1):

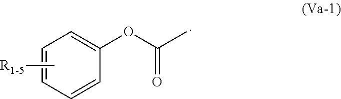

Embodiment B54: The stable composition of embodiment B50, wherein the compound has a moiety represented by the formula:

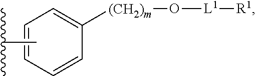

wherein:
subscript m is an integer from 0 to 2; and
L¹ is —C(O)—, —C(O)O—, —C(O)S—, or —C(O)NH—; and
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl.

Embodiment B55: The stable composition of embodiment B54, wherein the moiety is represented by formula:

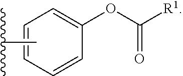

Embodiment B56: The stable composition of embodiment B54 or B55, wherein the moiety is represented by formula:

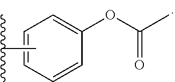

Embodiment B57: The stable composition of embodiment B50, wherein the compound is represented by formula (IIa-1a):

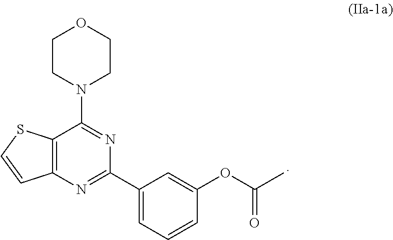 [SMILES: CC(=O)Oc1cccc(-c2nc(N3CCOCC3)c3sccc3n2)c1]

Embodiment B58: The stable formulation of any one of embodiments B50 to B57, prepared by sonicating a mixture of a) to d).

Embodiment B59: The stable formulation of any one of embodiments B50 to B58, wherein the one or more excipients are oleyl alcohol.

Embodiment B60: The stable formulation of any one of embodiments B50 to B59, wherein DMSO is present in an amount of from 0.1% to 20% by weight.

Embodiment B61: The stable formulation of embodiment B60, wherein DMSO is present in an amount of from 1% to 15% by weight of the base formulation.

Embodiment B62: The stable formulation of any one of embodiments B50 to B61, wherein the ratio of DMSO to oleyl alcohol is from 1:1 to 5:1 or from 1:1 to 3:1 by weight.

Embodiment B63: The stable formulation of embodiment B62, wherein the ratio of DMSO to oleyl alcohol is from 1:1 to 3:1 by weight.

Embodiment B64: The stable formulation of any one of embodiments B50 to B63, is a topical formulation.

### IX. Examples

### General Method for Preparation of Gel Formulations

The topical gel formulations of the present disclosure can be prepared according to the procedure provided below. Reaction conditions and steps not provided in the procedure below would be apparent to skilled practitioners.

The liquid excipients, for example DMSO when present, one or more excipients (e.g., an unsaturated fatty alcohol, an unsaturated fatty acid, an unsaturated fatty ester, an unsaturated fatty ether, or a combination thereof), an acid when present (e.g., citric acid), one or more solvents (e.g., C₁₋₃ alkyl-(OCH₂CH₂)₁₋₅—OH such as Transcutol P or 2-(2-ethoxyethoxy)ethanol, a C₂₋₆ alkylene glycol such as propylene glycol, a di-(C₂₋₆ alkylene) glycol such as dipropylene glycol, and a polyethylene glycol such as PEG400 when present) were mixed in a milliliter (mL) vial, optionally by vortex agitation. The active ingredient, a compound of formula (I), for example Compound 1.002 was then added, and the vial content was agitated by vortex for about 1 to 2 hours (e.g., 1.5 hours) or by sonication for about 10 to 30 minutes (e.g., 10 minutes) to dissolve the compound. The solution reached at a saturation concentration. A gelling agent, for example hydroxylpropyl cellulose (HPC) was added, and the vial was agitated by vortex for another ten minutes to obtain the gel formulation. The viscosity of the clear gel was measured, preferably to be in a range of about 7,000 to 10,000 centiPoise (cP) or about 10,000 to 20,000 centiPoise (cP).

Unless otherwise indicated, Nisso H was used as hydroxylpropyl cellulose (HPC) in examples.

### General Procedure for In Vitro Human Skin Permeation Study

The gel formulation including the compound of formula (I) (e.g., Compound 1.002) was used as the donor phase to determine the permeation of the tested compound (e.g., Compound 1.002) through human skin and its effectiveness as a topical application. Three skin donors and three diffusion cells (for each donor) per formulation were used in the in vitro skin permeation experiments. Split thickness dermatomed (approximately at 375 micrometers (μm) thickness) human cadaver skin, supplied by New York Firefighters Skin Bank, NY, was used to determine the permeation rate of the tested compound (i.e., the drug) in vitro. All in vitro skin permeation studies were conducted using the Vertical Diffusion Cells assembly with consoles (Model FDC-6), and heating controllers (Logan Instruments Inc. Somerset, N.J.). Each assembly consisted of six vertical, jacketed (37° C.±0.5° C.) 12-mL Franz diffusion cells with magnetic stirrer and 1.767 square centimeter (cm²) diffusion area.

Skin flux studies were run for a period of 48 hours. At predetermined intervals (2, 4, 8, 24, and 48 hours (h)) after starting the experiment, the entire contents of the receiver compartment were collected for determining concentrations of the drug by HPLC. The receiver compartment was refilled with fresh receiver medium. The receiver medium was pH 7.4 phosphate buffer with 0.5 milligrams per milliliter (mg/mL) of Oleath 20 with the saturation concentration of the drug in the receptor medium being 0.152 mg/mL. This solubility of the drug in the receiver medium was enough to ensure sink conditions throughout each collection interval.

Samples of the receptor phase were obtained at 2, 4, 8, 12, 24 and 48 h and concentrations of Compound 1.002 and 3-(4-morpholinothieno(3,2-d)pyrimidin-2-yl)phenol (metabolite of Compound 1.002, abbreviated as MTPP) were determined using a validated HPLC method. Without wishing to be bound by theory, it is believed that the metabolism of Compound 1.002 to MTPP takes place in the human skin by esterase enzymes.

HPLC method: Column—Gemini C-18 4.6×150 millimeters (mm), 5 μm particle size; Mobile Phase—water:acetonitrile 25:75 containing 0.1% trifluoroacetic acid (TFA); Flow Rate—1 milliliters per minute (mL/min); Detection—274 nanometers (nm); Column Temperature—40° C.; and Run Time—10 min. The retention times for MTPP and Compound 1.002 were 3.9 and 7.2 minutes, respectively.

### Accelerated Stability Study

Unless otherwise indicated, accelerated stability studies were performed at 80° C., because as a rule of thumb, 10 days at 80° C. (±2° C.) is equivalent to 2 years at room temperature.

The United States Food and Drug Administration (FDA) permits description of a formulation as having 18 months of product life at room temperature if the formulation is stable at 40° C. for a period of 3 months (90 days). The stability evaluation of a formulation under FDA prescribed storage conditions requires a period of 3 or 6 months. In order to shorten the study duration in formulation development, an accelerated stability is commonly evaluated and widely accepted in the field. Instability generally doubles for every 10° C. increase in temperature. Therefore, the accelerated stability at 80° C. for 5.6 days is expected to provide equivalent stability at room temperature for 18 months. Accordingly, the accelerated stability at 80° C. for 10 days is expected to provide equivalent stability at room temperature for at least 2 years.

The topical gel formulations including compound 1.002 were prepared in accordance with the procedure as follows: step 1) mixing of all excipients in a container; step 2) dissolving compound 1.002 in the mixture; and step 3) processing and homogenizing the mixture with a thickener to form a gel solution. The gel solution was left sitting at room temperature overnight to equilibrate before initiation of the hydrolytic stability experiment.

The gel solution was then dispensed into five 10-mL glass vials, labelled Day 0, Day 1, Day 3, Day 5, and Day 10. Day 0 sample was kept at room temperature, while Day 1, 3, 5, and 10 samples were stored in an 80° C. calibrated forced-air oven. Day 1 Sample was pulled out after one day storage in the oven and left at room temperate until performing an assay by HPLC; Day 3 Sample was pulled out after three days of storage and so on. All samples were analyzed by HPLC at the same time after ten days using the validated HPLC method as described herein.

### Example 1A: Accelerated Stability of Topical Formulations—pH, Buffer System, and/or EDTA

Various formulations according to Table 2 were studied in relation to effect of pH, type of acid used, system buffering, moisture, and use of chelating agents (e.g., ethylenediaminetetraacetic acid (EDTA)) on the accelerated stability of Compound 1.002. Compound 1.002 was present in an amount of 3.2% by weight of the base formulation in all studied formulations. Table 2 shows variety of conditions studied with noted formulations.

TABLE 2
Stability of Topical Formulations-Buffer Systems Study

| Ingredients/ID | 1A-1 | 1A-2 | 1A-3 | 1A-4 | 1A-5 | 1A-6 | 1A-7 |
|---|---|---|---|---|---|---|---|
| DMSO (wt/wt%) | 15 | 15 | 10 | 10 | 7.5 | 7.5 | — |
| Oleyl Alcohol | 5 | 3 | 4 | 3 | 4 | 3 | 4.5 |
| (wt/wt%) | | | | | | | |
| Transcutol P (wt/wt%) | 25 | 27 | 31 | 32 | 33.5 | 34.5 | 23 |
| DPG (wt/wt%) | 5 | 5 | 5 | 5 | 5 | 5 | 4.5 |
| PG (wt/wt%) | 50 | 50 | 50 | 50 | 50 | 50 | 45.5 |
| PEG400 (wt/wt%) | — | — | — | — | — | — | 22.5 |
| Total weight of the | 100 | 100 | 100 | 100 | 100 | 100 | 100 |
| base formulation | | | | | | | |
| Compound 1.002 | 3.2 | 3.2 | 3.2 | 3.2 | 3.2 | 3.2 | 3.2 |
| (wt/wt%) | | | | | | | |

Buffer System Studied

| | | | | | | | |
|---|---|---|---|---|---|---|---|
| acetic acid | — | x | x | x | x | x | x |
| citric acid | x | x | x | x | x | x | x |
| phosphoric acid | — | — | — | — | — | — | x |
| choline acetate | — | — | — | x | — | x | x |
| Tetramethylammonium | — | x | x | x | x | x | x |
| acetate | | | | | | | |
| soidum acetate | — | x | x | x | x | x | x |
| soidum citrate | x | x | x | x | x | x | x |
| sodium phosphate | — | — | — | — | — | — | x |
| EDTA disodium | — | — | — | — | — | x | — |

These studies indicate that pH, moisture, an acid (when buffered with a salt), buffering system (an acid/a salt), and/or EDTA do not appear to improve the accelerated stability of the gel formulations. Citric acid, when it is not buffered, showed some degree of effectiveness in reducing the hydrolysis of Compound 1.002 in formulations, in particular for the formulation containing no DMSO. Citric acid appears to provide some benefits in reducing the hydrolysis of Compound 1.002 in formulations. The effect provided by citric acid, when used alone, might be due to both effect of pH and/or its antioxidant properties.

### Example 2A: Accelerated Stability of Topical Formulations (without DMSO)—Effect of Citric Acid

The following formulation (without the gelling agent of HPC) was used as the control for the study, where Compound 1.002 was in about 1.6% by weight of the base formulation. The composition of the control formulation is provided in Table 3:

TABLE 3
Composition of control formulation 2A

| Ingredients/ID | 2A (Control) |
|---|---|
| Oleyl Alcohol (wt/wt %) | 4.5 |
| Transcutol P (wt/wt %) | 23 |
| DPG (wt/wt %) | 4.5 |
| PEG400 (wt/wt %) | 22.5 |
| PG (wt/wt %) | 45.5 |
| Total weight of the base formulation | 100.0 |
| Compound 1.002 (wt/wt %) | 1.6 |

Several formulations based on the control formulation were prepared with addition of citric acid in the amounts from 0.005% to 1% by weight of the base formulation. The pH of the formulations dropped from about 7.1 to about 3.33. The accelerated stability study at 80° C. showed that excellent stability was obtained when citric acid was included in the formulation in an amount of from about 0.01% to 0.1% by weight of the base formulation. The pH values of these formulations were between 4.96 and 4.07 at Day 0. The data are shown in Table 4.

TABLE 4
Stability of Gel Formulations (without DMSO) vs. Citric Acid

| Day/ID | 2A (control) | 2A-1 | 2A-2 | 2A-3 | 2A-4 | 2A-5 | 2A-6 |
|---|---|---|---|---|---|---|---|
| Citric Acid | 0 | 0.005 | 0.01 | 0.05 | 0.1 | 0.5 | 1 |
| (wt/wt%) | | | | | | | |

MTPP (%) From Hydrolysis of Compound 1.002 at 80° C.

| | | | | | | | |
|---|---|---|---|---|---|---|---|
| Day 0 | 0 | 0.0 | 0.0 | 0.0 | 0.0 | 0.0 | 0.0 |
| Day 3 | 14.30 | 2.51 | 1.51 | 0.96 | 1.08 | 9.64 | 9.11 |
| Day 5 | 17.03 | 4.09 | 2.56 | 1.17 | 1.64 | 15.45 | 13.57 |
| Day 7 | 18.80 | 4.81 | 3.67 | 1.82 | 1.87 | 12.67 | 17.98 |
| Day 10 | 20.21 | 6.97 | 4.92 | 3.97 | 2.77 | 14.10 | NA* |

pH Value of the Formulation

| | | | | | | | |
|---|---|---|---|---|---|---|---|
| Day 0 | 8.26 | 7.11 | 4.96 | 4.44 | 4.07 | 3.84 | 3.34 |
| Day 3 | nd | nd | nd | 4.68 | 4.53 | 3.37 | 3.40 |
| Day 5 | nd | nd | nd | 4.74 | 4.47 | 3.45 | 3.45 |
| Day 7 | 5.92 | 6.79 | 6.31 | 4.55 | 4.19 | 3.37 | 3.17 |
| Day 10 | 5.79 | 6.20 | 6.15 | 4.35 | 4.32 | 3.74 | NA* |

NA*—vial broke

### Example 3A: Accelerated Stability of Topical Formulations (with DMSO)—Effect of an Acid

Gel formulations with the following compositions were used for the study, where Compound 1.002 was included in the formulation in amounts of about 1.2%, 2.2%, or 2.3% by weight of the base formulation. The compositions of gel formulations 3A (without an acid) are included in Table 5:

TABLE 5
Composition of control formulation 3A

| Ingredients/ID | 3A |
|---|---|
| DMSO (wt/wt %) | 5 |
| Oleyl Alcohol (wt/wt %) | 5 |
| Transcutol P (wt/wt %) | 25 |
| DPG (wt/wt %) | 5 |
| PEG400 (wt/wt %) | 10 |
| PG (wt/wt %) | 50 |
| Total weight of the base formulation | 100.0 |
| HPC (wt/wt %) | 2 |
| Compound 1.002 (wt/wt %) | 1.2, 2.2, or 2.3 |

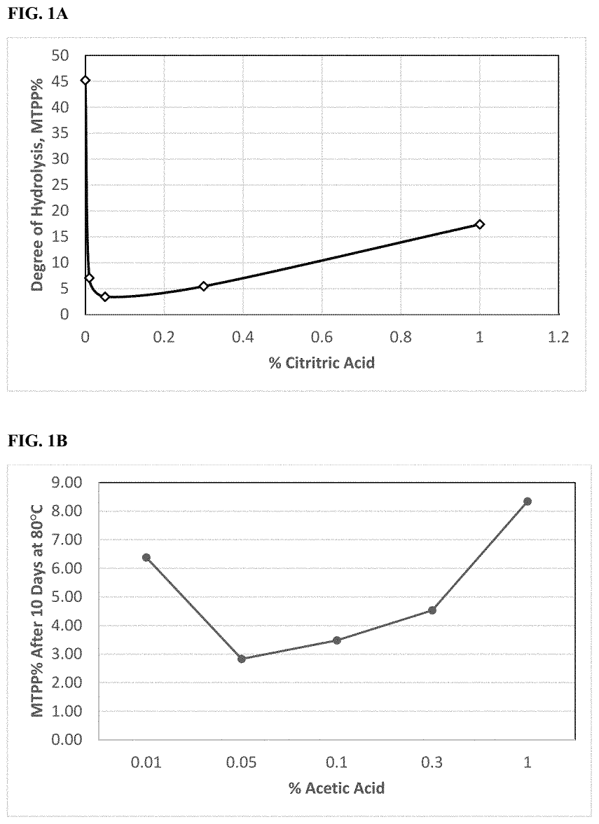

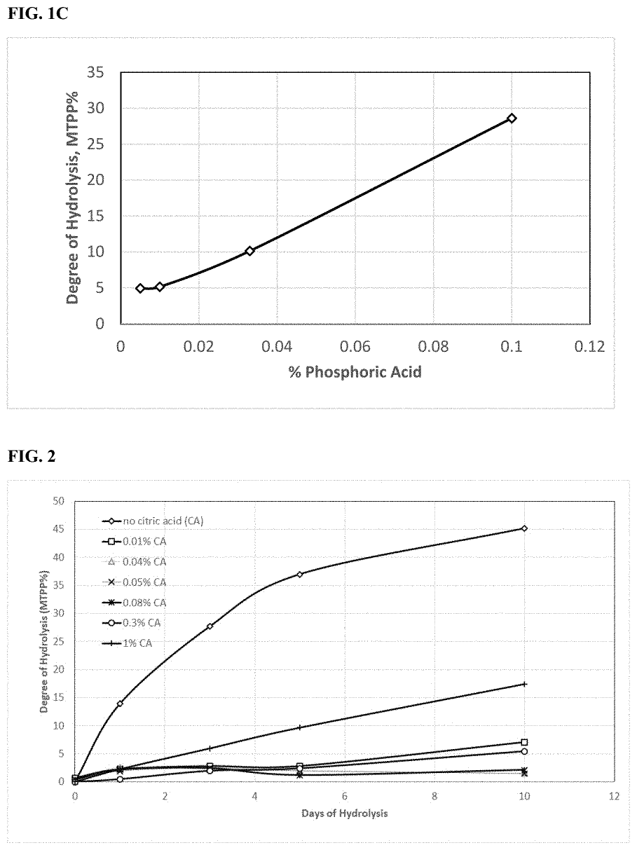

Several formulations based on the above compositions were prepared with addition of citric acid in an amount of from about 0.01% to 1% by weight of the base formulation. The formulations were prepared by mixing ingredients of DMSO, oleyl alcohol, Transcutol P, DPG, PRG400, PG, and citric acid (without sonication). The accelerated stability study at 80° C. showed that excellent stability was obtained when citric acid was in an amount of about 0.05% by weight of the base formulation. The data are shown in Table 6 and FIG. 1A.

TABLE 6
Stability of Gel Formulations (with 5% DMSO) vs. Citric Acid

| | | | 3A-3ca | | |
|---|---|---|---|---|---|
| Parameters/ID | 3A-1ca | 3A-2ca | (i.e., AA1ca) | 3A-4ca | 3A-5ca |
| Citric acid | 0.0 | 0.01 | 0.05 | 0.3 | 1.0 |
| (wt/wt %) | | | | | |
| Compound 1.002 | 1.2 | 1.2 | 2.3 | 1.2 | 1.2 |
| (wt/wt %) | | | | | |
| Nitrogen Purging Used | yes | yes | yes | yes | yes |

MTPP (%) From Hydrolysis of Compound 1.002 at 80° C.

| | | | | | |
|---|---|---|---|---|---|
| Day 0 | 0 | 0.70 | 0.40 | 0.00 | 0.05 |
| Day 1 | 13.9 | 2.26 | 0.76 | 0.50 | 2.3 |
| Day 3 | 27.7 | 2.81 | 0.98 | 1.98 | 5.99 |
| Day 5 | 37.0 | 2.82 | 2.66 | 2.42 | 9.66 |
| Day 10 | 45.2 | 7.07 | 3.43 | 5.45 | 17.4 |

pH Value of the Formulation

| | | | | | |
|---|---|---|---|---|---|
| Day 0 | — | 4.87 | — | 3.64 | 3.26 |
| Day 10 | — | 4.21 | — | 3.83 | 3.47 |

pH measurements: 10% formulation + 90% water

### Acetic Acid

Several formulations based on the above compositions were prepared with addition of acetic acid in the amounts from about 0.01% to 1% by weight of the base formulation. The formulations were prepared by mixing ingredients of DMSO, oleyl alcohol, Transcutol P, DPG, PRG400, PG, and acetic acid (without sonication). The accelerated stability study at 80° C. showed that excellent stability was obtained when acetic acid was in an amount of about 0.05% by weight of the base formulation. The data are shown in Table 7 and FIG. 1B.

TABLE 7
Stability of Gel Formulations (with 5% DMSO) vs. Acetic Acid

| | | 3A-2aa | | | |
|---|---|---|---|---|---|
| Parameters/ID | 3A-1aa | (i.e., AA1aa) | 3A-3aa | 3A-4aa | 3A-5aa |
| Acetic acid | 0.01 | 0.05 | 0.1 | 0.3 | 1.0 |
| (wt/wt %) | | | | | |
| Compound 1.002 | 1.2 | 2.2 | 1.2 | 2.2 | 2.2 |
| (wt/wt %) | | | | | |

MTPP (%) From Hydrolysis of Compound 1.002 at 80° C.

| | | | | | |
|---|---|---|---|---|---|
| Day 0 | 0.59 | 0 | 0.15 | 0 | 0 |
| Acetic acid | 0.01 | 0.05 | 0.1 | 0.3 | 1.0 |
| (wt/wt %) | | | | | |
| Day 1 | 3.54 | 1.8 | 0.88 | 0.43 | 1.09 |
| Day 3 | 4.52 | 2.36 | 1.67 | 1.42 | 2.34 |
| Day 5 | 3.26 | 1.81 | 3.02 | 1.80 | 3.27 |
| Day 10 | 6.38 | 2.83 | 3.48 | 4.53 | 8.34 |

pH Value of the Formulation

| | | | | | |
|---|---|---|---|---|---|
| Day 0 | 5.09 | 4.38 | 4.16 | 3.95 | 3.75 |
| Day 10 | 4.76 | 4.15 | 4.07 | 4.12 | 3.88 |

pH measurements: 10% formulation + 90% water

### Phosphoric Acid

Several formulations based on the above compositions were prepared with addition of phosphoric acid in the amounts from about 0.005% to 0.1% by weight of the base formulation. The formulations were prepared by mixing ingredients of DMSO, oleyl alcohol, Transcutol P, DPG, PRG400, PG, and phosphoric acid (without sonication). The accelerated stability study at 80° C. showed that excellent stability was obtained when phosphoric acid acid was in an amount of about 0.005% by weight of the base formulation. The data are shown in Table 8 and FIG. 1C

TABLE 8
Stability of Gel Formulations (with 5% DMSO) vs. Phosphoric Acid

| | 3A-1pa | | | |
|---|---|---|---|---|
| Parameters/ID | (i.e., AA1pa) | 3A-2pa | 3A-3pa | 3A-4pa |
| Phosphoric acid | 0.05 | 0.01 | 0.033 | 0.1 |
| (wt/wt %) | | | | |
| Compound 1.002 | 1.2 | 1.2 | 1.2 | 1.2 |
| (wt/wt %) | | | | |

MTPP (%) From Hydrolysis of Compound 1.002 at 80° C.

| | | | | |
|---|---|---|---|---|
| Day 0 | 0 | 0 | 0.17 | 0.1 |
| Day 1 | 1.07 | 0.90 | 1.46 | 3.32 |
| Day 3 | 1.72 | 2.27 | 3.46 | 9.75 |
| Day 5 | 1.47 | 2.77 | 6.65 | 15.6 |
| Day 10 | ND | 5.15 | 10.13 | 28.6 |
| Phosphoric acid | 0.05 | 0.01 | 0.033 | 0.1 |
| (wt/wt %) | | | | |

pH Value of the Formulation

| | | | | |
|---|---|---|---|---|
| Day 0 | — | 4.84 | 4.28 | 3.80 |
| Day 10 | — | 4.51 | 4.33 | 4.21 |

pH measurements: 10% formulation + 90% water; and ND - not determined.

### Example 4A: Accelerated Stability of Topical Formulations (with DMSO and Citric Acid)—Effect of Oleyl Alcohol

Several lots of TCI, Croda, and BASF oleyl alcohol were tested in various formulations containing citric acid in the amount of 0.04%, 0.05%, or 0.08% by weight of the base formulation. The formulations have the following compositions, as shown in Table 9.

TABLE 9
Compositions of formulations 4A-1ca to 4A-5ca

| Ingredients/ID | 4A-1ca to 4A-5ca |
|---|---|
| DMSO (wt/wt %) | 5 |
| Oleyl Alcohol (wt/wt %) | 5 |
| Transcutol P (wt/wt %) | 25 |
| DPG (wt/wt %) | 5 |
| PEG400 (wt/wt %) | 10 |
| PG (wt/wt %) | 50 |
| Total weight of the base formulation | 100.0 |
| Citric Acid (wt/wt %) | 0.04, 0.05, or 0.08 |
| HPC (wt/wt %) | 2 |
| Compound 1.002 (wt/wt %) | 1.2 |

The formulations were prepared by mixing ingredients of DMSO, oleyl alcohol, Transcutol P, DPG, PRG400, PG, and citric acid (without sonication). When citric acid was in an amount of about 0.05% by weight of the base formulation, the accelerated stability study at 80° C. showed that excellent stability obtained was neither affected by different sources of oleyl alcohol nor by air (e.g., with or without N₂ purging). The data are shown in Table 10.

TABLE 10
Stability of Gel Formulations (with DMSO and Citric Acid) vs.
Oleyl Alcohol/Air

| Parameters/ID | 4A-1ca | 4A-2ca | 4A-3ca | 4A-4ca | 4A-5ca |
|---|---|---|---|---|---|
| Citric acid (wt/wt %) | 0.05 | 0.05 | 0.05 | 0.04 | 0.08 |
| Oleyl Alcohol Batch | TCI | TCI | Croda | BASF | BASF |
| Nitrogen Purging Used | yes | no | no | yes | no |

MTPP (%) From Hydrolysis of Compound 1.002 at 80° C.

| | | | | | |
|---|---|---|---|---|---|
| Day 0 | 0.45 | 0.39 | 0.47 | 0.12 | 0.38 |
| Day 1 | 1.92 | 1.42 | 1.02 | 2.57 | 2.15 |
| Day 3 | 2.49 | 2.25 | 2.02 | 2.24 | 2.44 |
| Day 5 | 1.98 | 1.83 | 1.33 | 1.98 | 1.20 |
| Day 10 | 1.49 | 1.64 | 1.57 | 1.54 | 2.13 |

FIG. 2 shows the effect of citric acid in various amounts in the topical formulation containing 5% DMSO, according to combined data of Examples 3A (citric acid in 0%, 0.01%, 0.3% and 1% by weight) and Example 4A (citric acid in 0.04%, 0.05% (4A-1ca), and 0.08% by weight).

### Example 5A: Preparation of Gel Topical Formulation Including an Acid—Mixing

Gel formulation (AA1ca) containing 1.2% of compound 1.002 has the compositions as shown in Table 11:

TABLE 11
Composition of formulation AA1ca

| Ingredients/ID | AA1ca |
|---|---|
| DMSO (wt/wt %) | 5 |
| Oleyl Alcohol (wt/wt %) | 5 |
| Transcutol P (wt/wt %) | 25 |
| DPG (wt/wt %) | 5 |
| PEG400 (wt/wt %) | 10 |
| PG (wt/wt %) | 50 |
| Total weight of the base formulation | 100.0 |
| Citric Acid (wt/wt %) | 0.05 |
| HPC (wt/wt %) | 2 |
| Compound 1.002 (wt/wt %) | 1.2 |

Compositions (i.e., DMSO, oleyl alcohol, Transcutol P, DPG, PEG, PG, citric acid, and compound 1.002) of were mixed together at room temperature. The liquid formulation was then stirred in an open-air vessel (e.g., a beaker) at room temperature until all of compound 1.002 was dissolved, which took between 1 to 2 hours (e.g., about 1.5 hours). Then the gelling agent HPC was added and the mixture was blended until uniform. This process provided gel formulations with acceptable accelerated stability (e.g., MTPP (%) from hydrolysis of compound 1.002 at 80° C. at Day 10 was determined to be about 2.13%). The stability data are shown in Table 15 of Example 7A.

### Example 6A: Accelerated Stability of Topical Formulations (with DMSO)—Effect of Citric Acid in Heating/Mixing Processes

The effect of 0.05% citric acid on the hydrolysis of compound 1.002 to MTPP was determined, using the gel formulations having the compositions of Example 5A prepared by heating/mixing conditions. The corresponding gel formulation (6A) without citric acid was used as the control. The gel formulations were prepared as shown in Table 12:

TABLE 12
Preparation of formulations 6A, 6A-1, and 6A-2

| Formulation | |
|---|---|
| ID | Process |
| 6A (no citric acid) | 1) pretreating a premixture of DMSO and oleyl |
| | alcohol (OAl) at 45° C. (hotplate) for 1 h; |
| | 2) adding all other components including compound |
| | 1.002 at room temperature and then stirring the |
| | mixture; and |
| | 3) adding HPC and blending the mixture till |
| | uniform. |
| 6A-1 (0.05% citric | 1) pretreating a premixture of DMSO and oleyl |
| acid) | alcohol (OAl) at 45° C. (hotplate) for 1 h; |
| | 2) adding all other components including citric acid |
| | and compound 1.002 at room temperature and then |
| | stirring the mixture; and |
| | 3) adding HPC and blending the mixture till |
| | uniform. |
| 6A-2 (0.05% citric | 1) pretreating a premixture of DMSO, oleyl alcohol |
| acid) | (OAl), and citric acid (CA) at 45° C. (hotplate) for |
| | 1 h; |
| | 2) adding all other components including compound |
| | 1.002 at room temperature and then stirring the |
| | mixture; and |
| | 3) adding HPC and blending the mixture till |
| | uniform. |

Peroxide values and stability of compound 1.002 in the formulations were assessed, as shown in Table 13.

TABLE 13
Stability of Formulations by Heating/Mixing Processes vs. Citric Acid

| | Sample ID | | |
|---|---|---|---|
| | 6A (control) | 6A-1 | 6A-2 |
| | Pretreating DMSO, OAL, and/or CA | | |
| | DMSO/OAL | DMSO/OAL | DMSO/OAL/CA |
| Citric acid (wt/wt %) | 0.0 | 0.05 | 0.05 |
| Compound 1.002 | 12 | 12 | 12 |
| (mg/mL) | | | |
| Batch Size (g) | 506 | 506 | 506 |

Peroxide Values in Post formulation at 80° C.

| | | | |
|---|---|---|---|
| Day 0 | 2.61 | 2.19 | 1.45 |
| Day 1 | 1.30 | — | — |
| Day 3 | — | — | — |
| Day 5 | — | 1.78 | 1.74 |

MTPP (%) from hydrolysis at 80° C.

| | | | |
|---|---|---|---|
| Day 0 | 0.00 | 0.00 | 0.00 |
| Day 1 | 13.95 | 0.00 | 0.00 |
| Day 3 | 27.68 | 0.00 | 0.00 |
| Day 5 | 37.02 | 0.00 | 0.00 |
| Day 10 | 45.24 | 1.56 | 2.00 |

The above data indicate that citric acid plays an important role in stabilizing compound 1.002 in formulations prepared by heating/mixing process without sonication. The data suggest that excellent stability of the formulation can be obtained by a simple process when citric acid is present. For both formulations containing citric acid, regardless of the differences of processes, compound 1.002 did not show any instability at day 5 at 80° C. At day 10, the hydrolysis of compound 1.002 for these two formulations was determined to be 1.56% and 2%, respectively. In contrast, the formulation without citric acid, prepared by heating/mixing process (without sonication), provided unacceptable accelerated stability of compound 1.002.

### Example 7A: Preparation of Topical Formulation Including an Acid—Various Methods

Six formulations were prepared with compositions of Example 5A with the addition of 0.05% citric acid, i.e. formulation (AA1ca). The formulations were identical, but the processes were somewhat different as detailed in Table 14:

TABLE 14
Preparation of formulations 7A-1 to 7A-6

| Formulation | |
|---|---|
| ID | Process |
| 7A-1 | Example 5A |
| 7A-2 and | 1) Preheating each of DMSO and Oleyl alcohol (OAl) |
| 7A-3 | individually at 45° C. for 1 h (preheating/no premixing); |
| | 2) Adding citric acid (CA) and other liquid components |
| | and mixing; |
| | 3) Adding compound 1.002 and mixing till dissolved; and |
| | 4) adding HPC and blending the mixture till uniform. |
| 7A-4 | 1) Preheating each of DMSO, Oleyl alcohol (OAl), and |
| | citric acid (CA) individually at 45° C. for 1 h |
| | (preheating/no premixing); |
| | 2) Adding other liquid components and mixing; |
| | 3) Adding compound 1.002 and mixing till dissolved; and |
| | 4) adding HPC and blending the mixture till uniform. |
| 7A-5 | 1) Premixing DMSO and Oleyl alcohol (OAl) at room |
| | temperature for 1 h (premixing/no heating); |
| | 2) Adding citric acid (CA) and other liquid components |
| | and mixing; |
| | 3) Adding compound 1.002 and mixing till dissolved; and |
| | 4) adding HPC and blending the mixture till uniform. |
| 7A-6 | 1) Premixing DMSO and Oleyl alcohol (OAl), and citric |
| | acid (CA) and heating the premixture at 45° C. for 1 h |
| | (premixing/heating); |
| | 2) Adding citric acid (CA) and other liquid components |
| | and mixing; |
| | 3) Adding compound 1.002 and mixing till dissolved; and |
| | 4) adding HPC and blending the mixture till uniform. |

All processes did not use sonication, but employed low/or no temperature pretreatment of DMSO and oleyl alcohol with the subsequent addition of the other excipients and mixing at room temperature until all of compound 1.002 was dissolved. The gels from all six processes gave excellent accelerated stability results as shown in Table 15. The data are consistent with the stability profile of formulations containing 0.05% citric acid, as shown in Table 13 of Example 6A. The data suggest that all the mixing/temperature processes used to prepare formulation (AA1ca) provide very good hydrolytic stability of compound 1.002.

TABLE 15
Stability of Formulations by Various Processes

| Sample ID | | | | | |
|---|---|---|---|---|---|
| 7A-1 | 7A-2 | 7A-3 | 7A-4 | 7A-5 | 7A-6 |
| Process | | | | | |
| | DMSO/OAL | DMSO/OAL | DMSO/OAL/CA | DMSO/OAL | DMSO/OAL/CA |
| | (preheating/ | (preheating/ | (preheating/ | (premixing/ | (premixing/ |
| Ex. 5A | no premixing) | no premixing) | no premixing) | no heating) | heating) |

MTPP (%) from hydrolysis at 80° C.

| | | | | | | |
|---|---|---|---|---|---|---|
| Day 0 | 0.70 | 0.34 | 0.62 | 0.56 | 0.37 | 0.10 |
| Day 1 | 0.80 | 0.66 | 2.01 | 0.84 | 0.70 | 2.04 |
| Day 3 | 3.10 | 0.75 | 2.50 | 0.96 | 1.17 | 2.17 |
| Day 5 | 3.01 | 0.89 | 2.32 | 0.96 | 1.41 | 2.14 |
| Day 10 | 2.13 | 2.08 | 2.68 | 2.40 | 1.87 | 1.49 |

As can be seen, the process of Example 5A in an open-air vessel (e.g., a beaker) without nitrogen purging provided a convenient and robust process for preparing the present topical formulations, where the hydrolysis of compound 1.002 was controlled to be less than 10% (in particular less than 5%) under prescribed storage conditions (e.g., 10 days at 80° C.).

### Example 8A: Comparison of Accelerated Stability of Topical Formulations (with or without Citric Acid)

Citric acid approved to reduce the hydrolysis of compound 1.002. Stability of formulations with or without citric acid were directly compared and the results are shown in Table 16. This study further confirmed that the addition of 0.05% citric acid improved the stability of compound 1.002 in gel formulations.

TABLE 16
Direct Comparison of Stability of Gel Formulations
(with or without citric acid)

| | 8A-1 | 8A-2 | 8A-3 | 8A-4 |
|---|---|---|---|---|
| Ingredients/ID | (no CA) | (0.05% CA) | (no CA) | (0.05% CA) |
| DMSO (wt/wt %) | 5 | 5 | 5 | 5 |
| Oleyl alcohol (wt/wt %) | 5 | 5 | — | — |
| (BASF) | | | | |
| Oleyl alcohol (wt/wt %) | — | — | 5 | 5 |
| (Croda) | | | | |
| Transcutol P (wt/wt %) | 25 | 25 | 25 | 25 |
| DPG (wt/wt %) | 5 | 5 | 5 | 5 |
| PEG400 (wt/wt %) | 10 | 10 | 10 | 10 |
| PG (wt/wt) | 50 | 50 | 50 | 50 |
| Total weight of the | 100 | 100 | 100 | 100 |
| base formulation | | | | |
| Citric acid (wt/wt %) | — | 0.05 | — | 0.05 |
| HPC (wt/wt %) | 2 | 2 | 2 | 2 |
| Compound 1.002 (wt/wt %) | 3.0 | 3.0 | 3.0 | 3.0 |

MTPP (%) from hydrolysis at 80° C.

| | | | | |
|---|---|---|---|---|
| Day 0 | 0.00 | 0.00 | 0.00 | 0.00 |
| Day 5 | 1.75 | 0.87 | 2.07 | 1.50 |
| Day 10 | 4.08 | 1.88 | 4.22 | 1.79 |

### Example 9A: Skin Flux of Formulation (AA1ca)

Two formulations having the compositions shown in Table 17 were tested for skin permeation.

TABLE 17
Compositions of formulations 9A-1 and 9A-2

| | | 9A-2 |
|---|---|---|
| | 9A-1 | (i.e., AA1ca with |
| Ingredients/ID | (no citric acid) | 0.05% citric acid) |
| DMSO (wt/wt %) | 5 | 5 |
| Oleyl Alcohol (wt/wt %) | 5 | 5 |
| Transcutol P (wt/wt %) | 25 | 25 |
| DPG (wt/wt %) | 5 | 5 |
| PEG400 (wt/wt %) | 10 | 10 |
| PG (wt/wt %) | 50 | 50 |
| Total weight of the base formulation | 100.0 | 100.0 |
| Citric Acid (wt/wt %) | 0.0 | 0.05 |
| HPC (wt/wt %) | 2 | 2 |
| Compound 1.002 (wt/wt %) | 1.2 | 1.2 |

Formulation 9A-2 (i.e., AA1ca) was prepared by preheating DMSO/oleyl alcohol/citric acid for one hour at 45° C. (see the process for formulation 7A-4 of Example 7A). Formulation 9A-1 (with no citric acid) was prepared by a sonication process, i.e. sonicating the mixture of all ingredients (except for HPC) for 30 minutes, followed by the addition of HPC. Skin permeation studies were performed simultaneously for two studied formulations using the same human skin donor. As shown in Table 18, the skin permeation of compound 1.002 is not substantially affected by the inclusion of citric acid in the formulation.

TABLE 18
Average Cumulative Permeation of Compound 1.002 (μg/cm²)

| Hours/ | 9A-1 | 9A-2 (i.e., AA1ca with |
|---|---|---|
| Formulation ID | (no citric acid) | 0.05% citric acid) |
| 2 | 2.55 | 0.00 |
| 4 | 3.85 | 1.46 |
| 6 | 7.32 | 3.64 |
| 8 | 10.21 | 11.16 |
| 24 | 92.60 | 83.50 |
| 48 | 212.14 | 182.68 |

### Example 10A: Formulation (AA1ca)—Process for Preparing, Stability, and Skin Flux

Formulation (AA1ca) having the compositions as shown in Table 19:

TABLE 19
Composition of formulation (AA1ca)

| Ingredients/ID | AA1ca |
|---|---|
| DMSO (wt/wt %) | 5 |
| Oleyl Alcohol (wt/wt %) | 5 |
| Transcutol P (wt/wt %) | 25 |
| Dipropylene glyco (wt/wt %) | 5 |
| PEG400 (wt/wt %) | 10 |
| Propylene glycol (wt/wt %) | 50 |
| Total weight of the base formulation | 100.0 |
| Citric Acid (wt/wt %) | 0.05 |
| HPC (wt/wt %) | 1.8 |
| Compound 1.002 (wt/wt %) | 2.4 |

was prepared on a scale of 5, 20, or 50 kg, according to steps 1-9 as described below:

Step-1: Accurately weigh/dispense the following components, and place into separate, appropriate containers:
Compound 1.002 (Active Pharmaceutical Ingredient);
Dimethyl sulfoxide (DMSO);
Oleyl alcohol, super refined novol (e.g., highly purified);
Transcutol P (diethylene glycol monoethyl ether);
Dipropylene glycol;
PEG400 (e.g., Carbowax Sentry DR400);
Propylene glycol (e.g., DOW DR446);
Citric acid anhydrous (e.g., fine granular); and
Nisso H (hydroxypropyl cellulose).

Step-2: Add the DMSO and oleyl alcohol to the mixing vessel. Mix at 80 RPM under Nitrogen for 10 minutes.

Step-3: Add the Transcutol P (diethylene glycol monoethyl ether), dipropylene glycol, PEG400, and propylene glycol to the mixing vessel. Mix at 80 RPM under Nitrogen for 5 minutes.

Step-4: Add citric acid anhydrous to the mixing vessel. Mix at 175 RPM under Nitrogen for 5 minutes and verify that all solids have dissolved.

Step-5: Add compound 1.002 (API) to the mixing vessel. Mix at 175 RPM under Nitrogen for 60 minutes and verify that all solids have dissolved to form a homogenous mixture.

Step-6: Slowly add the Nisso HPC (i.e., Nisso H) to the mixing vessel. Mix at 175 RPM under Nitrogen for 22 hours.

Step-7: Homogenize the batch content using a rotor-stator homogenizer at 3,500 RPM for 45 minutes.

Step-8: Allow the gel to settle in the mixing vessel for 12 hours.

Step-9: Fill the 30-mL gel containers with the gel. Total containers filled are 156 bottles.

Stability of formulation (AA1ca) (e.g., 5 kg batch) prepared by the above process was shown in Table 20.

TABLE 20
Stability of Formulation (AA1ca)

| | | Month 1 | | | Month 2 | | |
|---|---|---|---|---|---|---|---|
| | T = 0 | 25° C. | 30° C. | 40° C. | 25° C. | 30° C. | 40° C. |
| Appearance | pass | pass | pass | pass | pass | pass | pass |
| Comp. 1.002 (%) | 100.3 | 99.6 | 99.2 | 99.0 | 99.3 | 99.0 | 98.1 |
| MTPP (%) | <0.05 | 0.61 | 0.66 | 1.64 | 1.97 | 2.51 | 2.86 |
| Color | 1 | 1 | 1 | 1 | 1 | 1 | 1 |
| Viscocity (cP) | 14,000 | 13,220 | 14,800 | 15,340 | 14,760 | 14,780 | 14,440 |
| Dissolution (%) | 89 | 89 | 90 | 89 | 89 | 89 | 88 |
| Moisture (%) | 0.20 | 0.27 | 0.28 | 0.23 | 0.28 | 0.33 | 0.46 |
| | | Month 3 | | | Month 3.5 | | |
| | T = 0 | 25° C. | 30° C. | 40° C. | 25° C. | 30° C. | 40° C. |
| Appearance | pass | pass | pass | pass | pass | pass | pass |
| Comp. 1.002 (%) | 100.3 | 99.1 | 98.9 | 98.1 | 98.9 | 98.7 | 98.6 |
| MTPP (%) | <0.05 | 1.26 | 1.45 | 2.18 | 1.32 | 1.69 | 2.35 |
| Color | 1 | 1 | 1 | 1 | 1 | 1 | 1 |
| Viscocity (cP) | 14,000 | 15,400 | 17,260 | 16,600 | 13,000 | 12,840 | 12,360 |
| Dissolution (%) | 89 | 87 | 89 | 88 | 86 | 88 | 88 |
| Moisture (%) | 0.20 | 0.27 | 0.28 | 0.34 | 0.54 | 0.31 | 0.42 |

Skin flux of formulation (AA1ca) (e.g., 5 kg batch) prepared by the above process was shown in Table 21.

TABLE 21
Average Cumulative Permeation of Compound 1.002 (μg/cm²)

| Hours/ | 10A-1 |
|---|---|
| Formulation ID | (i.e., AA1ca with 0.05% citric acid) |
| 2 | 3.2 |
| 4 | 7.8 |
| 8 | 12.0 |
| 24 | 67.0 |
| 48 | 261.2 |
| 72 | 426.1 |

### Example 1B: Accelerated Stability of Topical Formulations (without an Acid)—Effect of DMSO

### Study-1

Three formulations were prepared according to Table 22, where two formulations contain DMSO and one formulation contains no DMSO. All formulations also contained about the same amount of oleyl alcohol. The three formulations and results of the accelerated stability are shown in Table 22.

TABLE 22
Stability of Gel Formulations vs. DMSO

| Ingredients/ID | 1B-S1-1 | 1B-S1-2 | 1B-S1-3 |
|---|---|---|---|
| DMSO (wt/wt %) | 15 | — | 10 |
| Oleyl Alcohol (wt/wt %) | 5 | 4.5 | 4.5 |
| Transcutol P (wt/wt %) | 50 | 48 | 48 |
| DPG (wt/wt %) | 5 | 4.5 | 4.5 |
| PEG400 (wt/wt %) | — | 22.5 | 22.5 |
| PG (wt/wt %) | 25 | 20.5 | 10.5 |
| Total weight of the base formulation | 100.0 | 100.0 | 100.0 |
| HPC (wt/wt %) | 2 | 2 | 2 |
| Compound 1.002 (wt/wt %) | 3.2 | 1.6 | 2.5 |

MTPP (%) from hydrolysis at 80° C.

| | | | |
|---|---|---|---|
| Day 3 | 3.48 | 29.0 | 2.08 |
| Day 5 | 5.04 | 34.6 | 2.71 |
| Day 7 | 5.07 | 33.3 | 2.76 |

The conclusions that can be drawn from this study are a) DMSO at two different amounts by weight provides excellent stability when included in formulations that contain other excipients capable of causing instability of the formulations; and b) a substantially identical formulation that contains no DMSO shows significant hydrolytic instability of Compound 1.002 leading to the hydrolyzed product MTPP.

### Study-2

Very similar formulations were prepared with one of them containing 5% DMSO and the other three formulations containing no DMSO. All four formulations included the same excipients, oleyl alcohol, DPG, PG, PEG400 and Transcutol P. The composition of the formulations and the accelerated stability data are shown in Table 23.

TABLE 23
Stability of Gel Formulations vs. DMSO

| | ID | | | |
|---|---|---|---|---|
| | 1B-S2-1 | | | |
| Ingredients | (i.e., BA1-1) | 1B-S2-2 | 1B-S2-3 | 1B-S2-4 |
| DMSO (wt/wt %) | 5 | — | — | — |
| Oleyl Alcohol (wt/wt %) | 5 | 4.5 | 10 | 4.5 |
| Transcutol P (wt/wt %) | 25 | 23 | 23 | 23 |
| DPG (wt/wt %) | 5 | 4.5 | 4.5 | 4.5 |
| PEG400 (wt/wt %) | 10 | 22.5 | 20 | 22.5 |
| PG (wt/wt %) | 50 | 45.5 | 42.5 | 44.5 |
| Total weight of the base | 100.0 | 100.0 | 100.0 | 100.0 |
| formulation | | | | |
| HPC (wt/wt %) | 2 | 2 | 2 | 2 |
| Compound 1.002 (wt/wt %) | 3.0 | 1.6 | 3.1 | 1.68 |

MTPP (%) from hydrolysis at 80° C.

| | | | | |
|---|---|---|---|---|
| Day 0 | 0.0 | 0.0 | 0.0 | 0.0 |
| Day 5 | 2.35 | 10.75 | 10.55 | 10.56 |
| Day 10 | 3.10 | 16.46 | 17.42 | 15.19 |

Formulation (BA1) or (BA1-1) gave acceptable results where the hydrolysis of compound 1.002 was less than 10% under described storage condition. The other three formulations containing no DMSO gave the hydrolyzed product of MTPP, which exceeded 10% under described storage condition.

### Example 2B: Three Month Stability of Topical Formulations (without an Acid)—Effect of DMSO

Six formulations were prepared as shown in Table 24. The composition of the formulations was very similar, with the exception that three formulations contained DMSO and three formulations did not contain DMSO. All six formulations were tested for stability under the FDA approved accelerated condition of three months at 40° C. and 45% RH. The results are shown in

Table 25. All three time points the formulations containing DMSO had acceptable stability of less than 5% and the formulations without DMSO had unacceptable results of over 5%.

TABLE 24
Formulations Tested in the 3-Month Stability Study
(40 degrees C./75% RH)

| | 2B-1 | 2B-2 | | | | |
|---|---|---|---|---|---|---|
| | (i.e., | (i.e., | | | | |
| Ingredients/ID | BB1-1) | BA1-1) | 2B-3 | 2B-4 | 2B-5 | 2B-6 |
| DMSO (wt/wt %) | 15.0 | 5.0 | — | — | 15 | — |
| Oleyl Alcohol | 5.0 | 5.0 | 4.5 | 10.0 | 4.0 | 4.5 |
| (wt/wt %) | | | | | | |
| Brij 96/O-10 | — | — | — | — | 1.0 | 1.0 |
| Transcutol P | 25.0 | 25.0 | 23.0 | 23.0 | 25 | 23.0 |
| (wt/wt %) | | | | | | |
| DPG (wt/wt%) | 5.0 | 5.0 | 4.5 | 4.5 | 5 | 4.5 |
| PEG400 | — | 10.0 | 22.5 | 20.0 | — | 22.5 |
| (wt/wt %) | | | | | | |
| PG (wt/wt %) | 50.0 | 50.0 | 45.5 | 42.5 | 50 | 44.5 |
| Total weight of the | 100.0 | 100.0 | 100.0 | 100.0 | 100 | 100.0 |
| base formulation | | | | | | |
| HPC (wt/wt %) | 2.0 | 2.0 | 2.0 | 2.0 | 2.0 | 2.0 |
| Compound 1.002 | 3.2 | 3.0 | 1.6 | 3.1 | 2.5 | 1.7 |
| (wt/wt%) | | | | | | |
| Compd 1.002 | 2.56 | 2.40 | 1.28 | 2.48 | 2.00 | 1.34 |
| (80% saturation) | | | | | | |

TABLE 25
Data on the Hydrolysis of Compound 1.002 Under Accelerated Conditions
(40° C./75% RH)

| Batch No. | test parameters | 2B-1 | 2B-2 | 2B-3 | 2B-4 | 2B-5 | 2B-6 |
|---|---|---|---|---|---|---|---|
| Color | ASTM D1544 | 1 | 1 | 1 | 1 | 1 | 1 |
| Viscosity, cP | 30 rpm, s64 | 6,240 | 14,140 | 18,390 | 19,880 | 17,040 | 47,600* |
| | | | | | | | (6 rpm) |
| 1.002 mg/mL | TM0047 | 26.3 | 24.9 | 13.3 | 25.3 | 20.4 | 14.0 |
| MTPP, % | TM0047 | 0 | 0 | 0 | 0 | 0 | 0 |
| Moisture, % | KF | 2.9 | 1.4 | 0.7 | 0.8 | 2.3 | 2.8 |
| Crystals | Visual | No | No | No | No | No | No |

Month1

| | | | | | | | |
|---|---|---|---|---|---|---|---|
| Color | ASTM D1544 | 0 | 0 | 0 | 0 | 0 | 0 |
| Viscosity, cP | 30 rpm, s64 | 6,740 | 15,500 | 17,420 | 17,220 | 12,680 | 17,280 |
| 1.002, mg/mL | TM0047 | 23.86 | 22.28 | 11.72 | 22.57 | 18.47 | 12.5 |
| MTPP, % | TM0047 | 1.33 | 1.41 | 8.74 | 4.32 | 1.3 | 9.00 |
| Moisture, % | KF | 2.21 | 1.18 | 0.66 | 0.75 | 2.19 | 0.88 |
| Crystals | Visual | No | No | No | No | No | No |

Month 2

| | | | | | | | |
|---|---|---|---|---|---|---|---|
| Color | ASTM D1544 | 0 | 0 | 0 | 0 | 0 | 0 |
| Viscosity, cP | 30 rpm, s64 | 7,460 | 15,500 | 18,960 | 17,300 | 12,000 | 16,740 |
| 1.002, mg/mL | TM0047 | 23.72 | 22.28 | 11.82 | 22.74 | 18.61 | 12.49 |
| MTPP, % | TM0047 | 3.12 | 3.28 | 16.19 | 7.97 | 2.52 | 17.14 |
| Moisture, % | KF | 1.90 | 1.12 | 0.37 | 0.55 | 2.25 | 0.82 |
| Crystals | Visual | No | No | No | No | No | No |

Month 3:

| | | | | | | | |
|---|---|---|---|---|---|---|---|
| Color | ASTM D1544 | 0 | 0 | 0 | 0 | 0 | 0 |
| Viscosity, cP | 30 rpm, s64 | 11,240 | 14,980 | 18,240 | 16,180 | 12,600 | 16,150 |
| 1.002, mg/mL | TM0047 | 24.59 | 23.32 | 10.33 | 22.23 | 19.72 | 10.53 |
| MTPP, % | TM0047 | 4.35 | 4.64 | 20.63 | 10.62 | 3.57 | 22.84 |
| Moisture, % | KF | 2.95 | 1.07 | 0.68 | 0.44 | 2.02 | 0.65 |
| Crystals | Visual | No | No | No | No | No | No |

*Viscosity was measured using 6 rpm, as compared to others.

### Example 3B: Accelerated Stability of Topical Formulations Containing 5% DMSO Solution and No Acid—Effect of Oleyl Alcohol

Oleyl alcohol is believed to be the principal culprit in the instability of the formulations including compound 1.002. However, the other excipients also exhibited may contribute some negative effect. To determine the relative potency of each excipient, mixtures of 5% DMSO and 95% of each of the other excipients were prepared and tested for stability. The data is shown in Table 26. Oleyl alcohol was the worst performer, demonstrating and 6-fold to 10-fold higher hydrolysis than any of the other excipients. The other excipients provide acceptable levels of hydrolysis, even at this very high level in the composition.

TABLE 26
Stability of Gel Formulations vs. Oleyl Alcohol

| | ID | | | |
|---|---|---|---|---|
| Ingredients | 3B-1 | 3B-2 | 3B-3 | 3B-4 |
| DMSO (wt/wt %) | 5 | 5 | 5 | 5 |
| Oleyl Alcohol (wt/wt %) | — | — | — | 95 |
| Transcutol P (wt/wt %) | 95 | — | — | — |
| PEG400 (wt/wt %) | — | 95 | — | — |
| PG (wt/wt %) | — | — | 95 | — |
| Total weight of the base formulation | 100.0 | 100.0 | 100.0 | 100.0 |
| Compound 1.002 (wt/wt %) | 2.1 | 2.1 | 2.1 | 2.1 |

MTPP (%) from hydrolysis at 80° C.

| | | | | |
|---|---|---|---|---|
| Day 0 | 0.0 | 0.0 | 0.0 | 0.0 |
| Day 5 | 2.53 | 3.16 | 3.11 | 10.52 |
| Day 10 | 2.92 | 4.56 | 5.08 | 29.80 |

### Example 4B: Accelerated Stability of Topical Formulations (without an Acid)—Co-Effect of DMSO and Oleyl Alcohol

Six additional gel formulations with DMSO content of 15%, 10%, and 7.5% were prepared, as shown in Table 27. For each of these formulations two levels of oleyl alcohol (OAL) were included. For all formulations, the hydrolytic stability was better at the lower levels of oleyl alcohol. In general, the formulations containing higher amounts of DMSO showed lower levels of hydrolysis.

TABLE 27
Stability of Gel Formulations vs. DMSO/Oleyl Alcohol

| Ingredients/ID | 4B-1 | 4B-2 | 4B-3 | 4B-4 | 4B-5 | 4B-6 |
|---|---|---|---|---|---|---|
| DMSO (wt/wt %) | 15 | 15 | 10 | 10 | 7.5 | 7.5 |
| Oleyl Alcohol | 5 | 3 | 4 | 3 | 4 | 3 |
| (wt/wt %) | | | | | | |
| Transcutol P | 25 | 27 | 31 | 32 | 33.5 | 34.5 |
| (wt/wt %) | | | | | | |
| DPG (wt/wt%) | 5 | 5 | 5 | 5 | 5 | 5 |
| PG (wt/wt %) | 50 | 50 | 50 | 50 | 50 | 50 |
| Total weight of the | 100 | 100 | 100 | 100 | 100 | 100 |
| base formulation | | | | | | |
| HPC (wt/wt %) | 2 | 2 | 2 | 2 | 2 | 2 |
| Compound 1.002 | 3.2 | 3.2 | 3.2 | 3.2 | 3.2 | 3.2 |
| (wt/wt %) | | | | | | |

MTPP (%) from hydrolysis at 80° C.

| | | | | | | |
|---|---|---|---|---|---|---|
| Day 0 | 0.0 | 0.0 | 0.0 | 0.0 | 0.0 | 0.0 |
| Day 3 | 2.67 | 7.37 | 2.93 | 0.90 | 6.91 | 1.98 |
| Day 5 | 4.77 | 4.20 | 6.90 | 4.20 | 4.37 | 3.30 |
| Day 7 | 3.77 | 6.01 | 5.30 | 8.80 | 7.04 | 5.68 |
| Day 10 | 8.29 | 4.78 | 8.71 | 5.92 | 10.23 | 5.57 |

### Example 5B: Accelerated Stability of Topical Formulations (without an Acid)—Effect of DMSO/Oleyl Alcohol Ratio

Given the high ratio of DMSO (5%) to oleyl alcohol (95%) in Example 3B, additional studies were performed to determine the level of DMSO that is required to overcome the destructive effect of the peroxides in oleyl alcohol. Some of the ratios of DMSO to OAL were similar to those used in the examples discussed above and thus represent commercially acceptable ratios. Since oleyl alcohol contains peroxides, due to its double bond, it was decided to study the effect of DMSO on the peroxides that are originally present in oleyl alcohol in the formulations.

The effect of the ratio of DMSO to oleyl alcohol by weight in the composition was studied by measuring the level of peroxide and hydrolytic stability of compound 1.002 in the composition. The simple compositions included DMSO, oleyl alcohol (OAL), and 2.5% compound 1.002. Formulations (BA1) and (BA1-1) have 1:1 ratio of DMSO and OA1, which is represented by the composition of 50% DMSO in Table 28A; and Formulations (BB1) and (BB1-1) have 3:1 ratio of DMSO and OAL, which is represented by the composition of 75% DMSO in Table 28A. The effect of DMSO/OAL ratio on the level of peroxide is shown in Table 28A. The results show a continuous reduction of peroxide with an increased amount of DMSO as well as the duration of days at 80° C.

TABLE 28A
Effect of DMSO/Oleyl Alcohol Ratio on Peroxide Value

| | DSMO/OAL | | | | | |
|---|---|---|---|---|---|---|
| DMSO % | Ratio | Day 0 | Day 1 | Day 3 | Day 5 | Day 10 |
| 0 | 0.00 | 5.12 | 4.05 | 3.89 | 1.54 | 1.46 |
| 1 | 0.01 | 3.79 | 3.58 | 3.29 | 1.03 | 1.00 |
| 8 | 0.09 | 3.74 | 2.93 | 1.83 | 0.94 | 0.83 |
| 15 | 0.18 | 3.50 | 2.28 | 2.68 | 0.90 | 0.76 |
| 30 | 0.43 | 2.28 | 0.87 | 0.64 | 0.85 | 0.40 |
| 40 | 0.67 | 1.99 | 2.01 | 1.01 | 0.99 | 0.80 |
| 45 | 0.82 | 1.20 | 1.66 | 1.06 | 1.00 | 0.78 |
| 50 | 1.00 | 1.17 | 0.63 | 0.42 | 0.72 | 0.22 |
| 75 | 3.0 | 0.73 | 0.60 | 0.24 | 0.51 | 0.12 |

The hydrolysis of compound 1.002 increases as a function of the days that the formulation is kept under the accelerated conditions of 10 days at 80° C. As shown in Table 28B, at day 10, the hydrolysis of compound 1.002 was significantly reduced when the ratio of DMSO to OAL is from about 1:1 to about 3:1. The hydrolysis of compound 1.002 was observed substantially higher when DMSO is absent at any time point of days (Day 1 to Day 10).

TABLE 28B
Effect of DMSO/Oleyl Alcohol Ratio on
Hydrolysis of Compound 1.002

| | DSMO/OAL | | | | | |
|---|---|---|---|---|---|---|
| DMSO % | Ratio | Day 0 | Day 1 | Day 3 | Day 5 | Day 10 |
| 0 | 0.00 | 0.00% | 1.80% | 2.84% | 3.75% | 6.36% |
| 1 | 0.01 | 0.00% | 1.74% | 1.79% | 1.37% | 5.33% |
| 8 | 0.09 | 0.00% | 1.46% | 1.36% | 1.99% | 6.84% |
| 15 | 0.18 | 0.00% | 0.87% | 1.32% | 2.02% | 5.97% |
| 30 | 0.43 | 0.00% | 0.00% | 1.59% | 1.10% | 5.65% |
| 40 | 0.67 | 0.00% | 0.78% | 0.00% | 1.15% | 2.43% |
| 45 | 0.82 | 0.00% | 0.34% | 0.00% | 1.10% | 3.52% |
| 50 | 1.00 | 0.00% | 0.00% | 0.00% | 0.82% | 0.24% |
| 75 | 3.00 | 0.00% | 0.00% | 0.00% | 0.39% | 0.00% |

The above studies provide the following key findings: 1) peroxide value decreases with an increased amount of DMSO and increased numbers of days at 80° C.; 2) hydrolysis of compound 1.002 increases with increased numbers of days at 80° C. even at the presence of 30% DMSO/70% OAL; 3) at ratios of 1:1 and 3:1 DMSO:OAL, the formulations provide substantially reduced hydrolysis product (i.e., <1% on day 5 or day 10); and 4) the peroxide value at Day 0 in the formulations provides an excellent indicator to predicate the hydrolysis of compound 1.002 under 2-year ambient storage conditions.

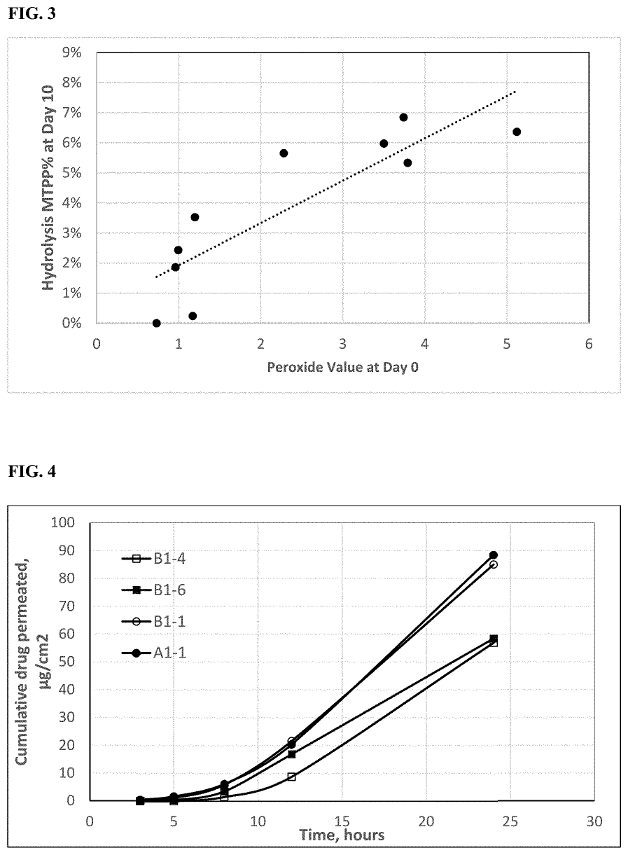

FIG. 3 shows the effect of the peroxide value immediately upon the preparation of the formulation (on Day 0) on the accelerated hydrolytic stability of Compound 1.002 (10 day/80° C.). As noted above, stability for 10 days at 80° C. is generally considered to be equivalent to stability for 2 years at room temperature. Therefore, the long-term stability of topical formulations including compound 1.002 can be determined at the time of the formulation is initially prepared according to the peroxide index as determined.

### Example 6B: Accelerated Stability of Topical Formulations (without an Acid)—Effect of Excipients Other than Oleyl Alcohol

As shown in Example 3B, hydrolysis of compound 1.002 was observed even at the absence of oleyl alcohol. To determine the effect of each of the excipients on hydrolysis of compound 1.002, five gel formulations were prepared as shown in Table 29, where each formulation only included one excipient, i.e. DMSO, PEG400, or Transcutol P. A formulation including a 1:1 ratio of DMSO to PEG400 was also investigated.

TABLE 29
Stability of Gel Formulations vs. Excipients (Other than Oleyl
Alcohol)

| | ID | | | | |
|---|---|---|---|---|---|
| Ingredients | 6B-1 | 6B-2 | 6B-3 | 6B-4 | 6B-5 |
| DMSO (wt/wt %) | 100 | — | 50 | — | — |
| PEG400 (wt/wt %) (BASF) | — | 100 | 50 | — | — |
| PEG400 (wt/wt %) (Croda) | — | — | — | 100 | — |
| Transcutol P (wt/wt %) | — | — | — | — | 100 |
| Total weight of the base | 100 | 100 | 100 | 100 | 100 |
| formulation | | | | | |
| HPC (wt/wt %) | 2 | 2 | 2 | 2 | 2 |
| Compound 1.002 (wt/wt %) | 11.1 | 11.1 | 11.1 | 3.0 | 11.1 |

MTPP (%) from hydrolysis at 80° C.

| | | | | | |
|---|---|---|---|---|---|
| Day 0 | 0.00 | 0.00 | 0.00 | 2.48 | 0.00 |
| Day 3 | 0.10 | 3.59 | 0.36 | 6.03 | 15.10 |
| Day 5 | 0.14 | 4.17 | 0.26 | nd | 16.70 |
| DAY 5 Moisture % | 0.31 | 0.67 | 0.46 | nd | nd |
| Day 7 | 0.11 | 9.27 | 0.33 | 12.19 | 15.46 |
| Day 10 | 0.14 | 7.38 | 0.33 | 10.93 | 27.27 |

The study suggests that a) DMSO alone provides excellent stability and by far the best among all excipients tested; b) the use of DMSO in combination with an excipient that causes instability, counteracts that instability and provides excellent stability of the combination gel (i.e., 6C); c) PEG400 as the single excipient in the gel formulation increases the level of instability; and d) Transcutol P as the single excipient in the gel formulation (other than OAL) provides the worst stability of any of the excipients tested.

### Example 7B: In Vitro Human Skin Permeation Studies—Formulations (BA1-1), (BB1-1), (BB1-4), and (BB1-6)

Four formulations (i.e., Formulations (BA1-1), (BB1-1), (BB1-4), and (BB1-6)) containing compound 1.002 were prepared with different amounts of DMSO and OAL as shown in Table 30. The skin permeation data are shown in FIG. 4. The results indicate that the skin permeation values of the two formulations containing 5% OAL were about the same over a 24 hour period and these values were about 30 to 50% higher than that of the two formulations containing 3% OAL.

TABLE 30
Gel Formulations Containing 3% or 5% Oleyl Alcohol

| | ID | | | |
|---|---|---|---|---|
| | 7B-1 | 7B-2 | 7B-3 | 7B-4 |
| | Formulation | | | |
| Ingredients | BA1-1 | BB1-4 | BB1-6 | BB1-1 |
| DMSO (wt/wt %) | 5 | 10 | 7.5 | 15 |
| Oleyl Alcohol (wt/wt %) | 5 | 3 | 3 | 5 |
| Transcutol P (wt/wt %) | 25 | 32 | 34.5 | 25 |
| DPG (wt/wt %) | 5 | 5 | 5 | 5 |
| PEG400 (wt/wt %) | 10 | — | — | — |
| PG (wt/wt %) | 50 | 50 | 50 | 50 |
| Total weight of the base formulation | 100.0 | 100.0 | 100.0 | 100.0 |
| HPC (wt/wt %) | 2 | 2 | 2 | 2 |
| Compound 1.002 (wt/wt %) | 3.0 | 3.2 | 3.2 | 3.2 |

### Example 8B: In Vitro Human Skin Permeation Studies (without an Acid)—Effect of Oleyl Alcohol

The skin permeation of Formulations (BA1-1) and (BB1-1) of Example 7B was compared to two other formulations containing 2% and 3% oleyl alcohol, respectively. The four formulations and the skin permeation results are shown in Table 31.

TABLE 31
Skin Permeation vs. Oleyl Alcohol

| | ID | | | |
|---|---|---|---|---|
| | | | 8B-3 | 8B-4 |
| Ingredients | 8B-1 | 8B-2 | (i.e., BB1-1) | (i.e., BA1-1) |
| DMSO (wt/wt %) | 5 | 5 | 15 | 5 |
| Oleyl Alcohol (wt/wt %) | 3 | 2 | 5 | 5 |
| Transcutol P (wt/wt %) | 25 | 25 | 25 | 25 |
| DPG (wt/wt %) | 5 | 5 | 5 | 5 |
| PEG400 (wt/wt %) | 10 | 10 | — | 10 |
| PG (wt/wt %) | 50 | 50 | 50 | 50 |
| Total weight of the base | 100.0 | 100.0 | 100.0 | 100.0 |
| formulation | | | | |
| HPC (wt/wt %) | 2 | 2 | 2 | 2 |
| Compound 1.002 (wt/wt %) | 3.1 | 3.1 | 3.0 | 3.0 |

Average Cumulative Compound 1.002 Permeated, μg/cm²

| | | | | |
|---|---|---|---|---|
| 3 hours | 0.7 | 0.6 | 0.2 | 0.3 |
| 5 hours | 2.3 | 1.5 | 0.5 | 0.4 |
| 8 hours | 3.3 | 5.6 | 2.1 | 1.5 |
| 12 hours | 9.3 | 13.5 | 5.2 | 4.1 |
| 24 hours | 50.4 | 65.5 | 29.5 | 38.7 |

The skin permeation was performed using three diffusion cells per formulation. The results show that the skin permeation values from the two formulations containing 5% oleyl alcohol were 30% to 50% higher than those from the formulations containing 3 or 2% oleyl alcohol.

### Example 9B: In Vitro Human Skin Permeation Studies—Formulations (BA1-1) and (BB1-1)

Formulations (BA1-1) and (BB1-1) of Example 7B were studied for in vitro skin permeation through three different human skins using skin diffusion cells as described herein.

Four diffusion cells per skin per formulation were used for a total of 24 skin diffusion cells. The average permeation of compound 1.002 in all skin diffusion cells (12 cells for each formulation) was very similar between Formulations (BA1-1) and (BB1-1), as shown in Table 32

TABLE 32
Skin Permeation of Formulations (BA1-1) and (BB1-1) (μg/cm²)

| | Average Permeation of Compound 1.002 | |
|---|---|---|
| | Through Three Human Skins (μg/cm²) | |
| Hours | BA1-1 | BB1-1 |
| 1 | 0.27 | 0.00 |
| 3 | 2.59 | 0.79 |
| 5 | 8.84 | 4.24 |
| 8 | 23.99 | 15.94 |
| 24 | 102.76 | 99.11 |
| 48 | 196.46 | 198.96 |

### Example 10B: Process for Preparing Topical Formulations (without an Acid)—By Sonication

All the excipients of formulation (BA1-1) (see ingredients of Example 7B) were mixed together including compound 1.002, except for HPC. The liquid formulation was then sonicated until all of compound 1.002 is dissolved, which took between 30 and 50 minutes. Then HPC was added and the mixture was blended until uniform. Peroxide values and viscosity were used as process parameters. This process provided low peroxide values and in subsequent studies it was shown to provide gel formulations with acceptable accelerated stability (i.e., hydrolysis of compound 1.002 is less than 10% under prescribed storage conditions).

### Example 11B: Preparation of Topical Formulation (BA1-1)—Sonication Vs Other Methods

### Study 1: Using Mixing all Ingredients Under Helium Blanket

In a 400-mL glass disposable beaker, all liquid components were weighted, as to prepare a final formulation containing 150 mL of gel formulation. Marine-type propeller/mixer was used continuously at about 200 rpm for 30 minutes. The beaker was covered with parafilm.

The saturation concentration of compound 1.002 in the formulation was determined in previous studies. The amount of compound 1.002 needed for the formulation was calculated to provide the formulations with 80% or 100% degree to saturation as required. The predetermined amount of compound 1.002 was then added into the beaker and the beaker was again sealed with parafilm. The contents in the beaker were mixed, using a mixing equipment until all compound was dissolved (approximate 30 min). HPC was then added to thicken the solution with mixing at speed of 300-400 rpm, for about 30 minutes. The solution was allowed to sit at room temperature overnight.

After the mixing was completed, the contents were poured into glass vials and the vials were capped tightly.

The above prepared formulations are found to contain peroxides at much higher values.

### Study 2: Sonication DMSO/OAL Followed by Mixing of Other Ingredients

DMSO and oleyl alcohol was mixed and sonicated for about 10 to 40 minutes. The sonicated mixture was blended with the other ingredients including compound 1.002 in a 400 rpm mixing process and finally HPC was blended in to provide formulations. Peroide vales and stability of compound 1.002 in the formulation were accessed, as shown in Table 33.

TABLE 33
Peroxide Values and Stability of Formulations by Study 2

| | Sample ID | | | |
|---|---|---|---|---|
| | 11B-52-1 | 11B-52-2 | 11B-52-3 | 11B-52-4 |
| Pre-sonicating | No sonication | 10 min | 30 min | 50 min |
| DMSO/OAl | | | | |
| Peroxide Value | 3.30, 2.16 | 1.96 | 1.14 | 2.26 |
| after pretreat | | | | |

Peroxide Values in Post formulation at 80° C.

| | | | | |
|---|---|---|---|---|
| Day 0 | — | 1.84 | 1.92 | 4.97, 5.01 |
| Day 1 | — | 0.36 | 0.37 | 3.06 |
| Day 3 | — | 1.58 | 1.68 | 1.80 |

MTPP (%) from hydrolysis at 80° C.

| | | | | |
|---|---|---|---|---|
| Day 0 | — | 0.00 | 0.00 | 0.00 |
| Day 1 | — | 1.73 | 2.56 | 1.80 |
| Day 3 | — | 3.92 | 5.12 | 3.08 |
| Day 5 | — | 7.61 | 9.27 | 5.71 |
| Day 10 | — | — | — | — |
| Day 19 | — | 11.90 | 17.90 | 6.41 |

### Study 3: Sonication of DMSO/OA1 Followed by Blending all Other Ingredients and Sonicating Again

This study was preformed to demonstrate if the stability of formulations were improved by eliminating oxygen in all excipients by sonication. As shown in Table 34, the hydrolysis of compound 1.002 was substantially reduced. The data suggest that removing both the peroxides in oleyl alcohol and the oxygen dissolved in formulations significantly improved the stability of the formulation.

TABLE 34
Peroxide Values and Stability of Formulations by Study 3

| | Sample ID | |
|---|---|---|
| | 11B-53-1 | 11B-53-2 |
| Pre-sonicating DMSO/OAL | 10 min | 30 min |
| Peroxide Value after pretreat | 2.10 | 1.00 |

Peroxide Values in Post formulation at 80° C.

| | | |
|---|---|---|
| Day 0 | 2.15 | 2.34 |

MTPP (%) from hydrolysis at 80° C.

| | | |
|---|---|---|
| Day 0 | 1.74 | 0.00 |
| Day 1 | 0.00 | 1.74 |
| Day 3 | 1.89 | 1.51 |
| Day 5 | 1.91% | 0.00 |
| Day 10 | 3.07 | 2.14 |

### Study 4: Exclusion of Oxygen and Water

All ingredients, including compound 1.002, were placed in a closed vessel and put on rollers overnight. It was made certain that there was no air space in the vessel and the vessel was tightly closed to prevent oxygen and water entering the vessel. As shown in Table 35, the hydrolysis of compound 1.002 was high. The study suggests that, without sonication, the hydrolysis of compound 1.002 excessively high even when water and oxygen are excluded.

TABLE 35
Peroxide Values and Stability of Formulations by Study 4

| | Sample ID | |
|---|---|---|
| | 11B-54-1 | 11B-54-2 |
| Peroxide Value after roll-mixing | 0.06, 0.10 | 0.23 |
| all ingredients overnight | | |

Peroxide Values in Post formulation at 80° C.

| | | |
|---|---|---|
| Day 0 | 0.06, 0.10 | 0.23 |

MTPP (%) from hydrolysis at 80° C.

| | | |
|---|---|---|
| Day 0 | 0.78% | 0.00 |
| Day 1 | 19.78 | 24.35% |
| Day 3 | 49.66% | 43.60% |
| Day 5 | 56.52% | — |

### Study 5: Pre-Treatment of DMSO/OAL at 80° C. for Extended Period of Time (Days)

It was previously observed that the peroxide value and the hydrolysis were reduced when DMSO and oleyl alcohol were heated together. To confirm this observation, DMSO and oleyl alcohol were mixed and pretreated at 80° C. for up to 9 days. The peroxide value did not seem to radically change, but the hydrolysis of compound 1.002 appeared to be reduced as a function of days that the DMSO-OA1 mixture was kept at 80° C., as shown in Table 36. It is believed that the high temperature not only destroys the peroxides present in oleyl alcohol, but also allows oxygen to react with oleyl alcohol to form peroxides, which are subsequently quenched by DMSO at the elevated temperature.

TABLE 36
Peroxide Values and Stability of Formulations by Study 5

| | Sample ID | | | | |
|---|---|---|---|---|---|
| | 11B-55-1 | 11B-55-2 | 11B-55-3 | 11B-55-4 | 11B-55-5 |
| Pre-treat | 1 day | 5 days | 7 days | 8 days | 9 days |
| DMSO/OAl at | | | | | |
| 80° C. | | | | | |
| Peroxide Value | 4.33 | ND | 4.52 | 4.61 | 9.81, 8.35 |
| after pretreat | | | | | |

Peroxide Values in Post formulation at 80° C.

| | | | | | |
|---|---|---|---|---|---|
| Day 0 | 1.89 | 2.56 | — | — | 1.25 |
| Day 1 | 1.98 | 1.54 | — | — | 1.36 |
| Day 3 | 3.37 | ND | — | — | — |
| Day 5 | 2.76 | ND | — | — | — |
| Day 10 | ND | ND | — | — | — |

MTPP (%) from hydrolysis at 80° C.

| | | | | | |
|---|---|---|---|---|---|
| Day 0 | 0.00% | ND | — | | 0.00 |
| Day 1 | 4.44% | 0.00% | — | | 0.00 |
| Day 3 | 9.18% | 2.23% | — | — | 0.00 |
| Day 5 | 10.63% | 3.15% | — | — | 0.65% |
| Day 10 | 17.92% | 5.39% | — | — | 0.2% |
| | | | | | (Day 11) |

Study 6: Repeat Study 5 with Shortened Process Time

This experiment was conducted to validate the results shown in Study 5 and to determine if the processing time could be reduced below 10 days at 80° C. As shown in Table 37, the hydrolysis of compound 1.002 was acceptable even at 6 days at 80° C.

TABLE 37
Peroxide Values and Stability of Formulations by Study 6

| | Sample ID | | | | |
|---|---|---|---|---|---|
| | 11B-56-1 | 11B-56-2 | 11B-56-3 | 11B-56-4 | 11B-56-5 |
| Pre-treat | 1 day | 3 days | 6 days | 7 days | 8 days |
| DMSO/OAl at | | | | | |
| 80° C. | | | | | |
| Peroxide Value | 4.39 | 11.6, 11.5 | 13.66 | 16.02 | ND |
| after pretreat | | | | | |

Peroxide Values in Post formulation at 80° C.

| | | | | | |
|---|---|---|---|---|---|
| Day 0 | — | — | — | — | 5.77 |
| Day 1 | — | — | 2.40 | — | — |

MTPP (%) from hydrolysis at 80° C.

| | | | | | |
|---|---|---|---|---|---|
| Day 1 | — | — | 0.00 | 0.00 | 0.00 |
| Day 3 | — | — | 0.00 | 0.00 | 0.00 |
| Day 5 | — | — | 0.00 | 0.00 | 2.27 |
| Day 10 | — | — | 1.69 | 1.44 | 2.70 |

Study 7: Purging with Nitrogen

Several experiments were performed using purging with nitrogen to determine if eliminating the oxygen dissolved in the excipients would eliminate or minimize the hydrolysis of compound 1.002 to MTPP. This study did not involve any process, such as sonication, high temperature or other processes that would reduce peroxides. The hydrolysis of compound 1.002 was observed extensively to MTPP in an amount of from 8.6% to 30% at Day 1 and from 12% to 41% at Day 3. This study suggests that the peroxides from oleyl alcohol present in the formulation are the prime causes of the hydrolysis of compound 1.002 to MTPP.

### Example 12B: Skin Flux of Formulation (BA1-1) by Different Processes

Formulation (BA1-1) produced by two different processes were tested for skin permeation. The two formulations were A) produced by the preheat process, i.e. preheat DMSO/Oleyl alcohol at 80° C. for 10 days; and B) produced by the sonication process, i.e. sonicating the mixture of all ingredients (except for HPC) for 30 minutes. Skin permeation studies were performed simultaneously for two studied formulations using the same human skin donor. As shown in Table 38, the skin permeation of formulation (BA1-1) is not affected by these two processes.

TABLE 38
Average Cumulative Permeation of Compound 1.002 (μg/cm²)

| | Formulation ID | |
|---|---|---|
| Hours | 12B-1 | 12B-2 |
| 2 | 0.00 | 2.55 |
| 4 | 1.30 | 3.85 |
| 6 | 1.60 | 7.32 |
| 8 | 4.25 | 10.21 |
| 24 | 84.32 | 92.60 |
| 48 | 228.56 | 212.14 |

### Example 13: Dermal Tolerability and Toxicity Study in Minipigs

A study was performed to assess the dermal tolerability and toxicity of two different formulations (without an acid) in minipigs (i.e., one of formulations is formulation (BA1)). The formulations were administered daily by dermal application for at least 20 hours to the minipigs for 4 weeks (28 days). Six groups of minipigs (2/sex/group, aged 2-4 months with body weight between 6.4-9.0 kilograms (kg) for males and 7.1-9.1 kg for females) were selected with each group receiving one formulation. The compositions of two formulations are summarized in Table 39.

TABLE 39
Compositions of formulations (13-1) and (13-2)

| | ID | |
|---|---|---|
| | 13-1 | 13-1 |
| Ingredients | (i.e., BA1 formulation) | (without DMSO) |
| DMSO (wt/wt %) | 5 | — |
| Oleyl Alcohol (wt/wt %) | 5 | 10 |
| Transcutol P (wt/wt %) | 25 | 23 |
| Dipropylene glycol (wt/wt %) | 5 | 4.5 |
| PEG400(wt/wt %) | 10 | 20.0 |
| Propylene glycol (wt/wt %) | 50 | 42.5 |
| Total weight of the base | 100.0 | 100.0 |
| formulation | | |
| HPC (wt/wt %) | 2 | 2 |
| Compound 1.002 (wt/wt %) | 1.84 | 2.55 |

Formulations were stored at controlled room temperature (20° C.±5° C.).

Each minipig served as its own control with one application site administered a placebo gel formulation while the contralateral application site was administered a formulation including compound 1.002 gel at a concentration that corresponded to approximately 80% of the maximum saturation for each formulation. The application site was dosed with 2 mg/cm² (each site=5% body surface area; for example: 40 mg formulation/kg body weight corresponding to approximately 2 mg formulation/cm² based on a minipig of 10 kg). The dose volume was between about 35-39 microliters per kilogram (μL/kg) based on a dose volume of 40 milligrams per kilogram (mg/kg) and the density of the formulations. Minipigs were subjected to 20 hours of application with a porous gauze covering the application site; formulations were wiped off after 20 hours and dermal observations were conducted 4 hours later.

Criteria for evaluation included survival, clinical observations, application site observations, body weights, food consumption, macroscopic assessment of the application sites, and punch biopsies collected for histologic evaluation of the application sites. Toxicokinetics were evaluated from animals dosed with formulation (13-1).

All animals survived the study and no treatment related effects were noted on non-clinical observations, body weights, or food consumption. Local reaction site observations were generally similar between placebo gel and formulations containing compound 1.002 with varying levels of severity. These included very slight to severe erythema, scaly skin, and brown and/or red spots. Therefore, the dermal findings were considered the consequence of the placebo gel formulations and not compound 1.002, although a slight exacerbation of these findings in the presence of compound 1.002 could not be ruled out.

Specifically, animals treated with formulation (13-1) generally demonstrated the fewest number of observations and erythema was very slight in all animals with one animal demonstration well-defined erythema. In contrast, formulation (13-2) was not tolerated with some animals noted with moderate to severe erythema. Across all dose groups, erythema was no longer present at 1-2 weeks after treatment stopped, while the other dermal observations remained. Microscopic evaluation of placebo gel and compound 1.002-containing gel treated skin from all animals demonstrated multifiocal or diffuse epidermal hyperplasia associated with hyperkeratosis and minimal to slight subepidermal mononuclear cell infiltration. Similar to the dermal observations, animals treated with formulation (13-1) demonstrated the lowest severity (minimal) of hyperplasia with one placebo gel and one compound 1.002-treated application site showing no findings; and the majority of these application sites did not have cellular infiltration. In contrast, the most severely affected animals were treated with the formulation (13-2) and had minimal to moderate hyperplasia of the application sites.

Levels of compound 1.002 and its corresponding compound of formula (IV) (e.g., formula (IVa)) were below the LLOQ in all blood samples from formulation (13-1) treated animals.

This study indicated that dermal application of two formulations to minipigs for up to 28 days (application time of 20 hours/day) were generally tolerated. Varying degrees of erythema, other dermal observations, and epidermal hyperplasia with some mononuclear cell infiltration were noted across both placebo and compound 1.002-containing formulations. Within each group, findings were generally similar between the application sites treated with placebo or compound 1.002-containing formulations, suggesting the findings were related to the placebo formulation components and not compound 1.002, although a slight exacerbation of these findings in the presence of compound 1.002 could not be ruled out. Formulation (13-1) demonstrated the most acceptable tolerability profile and did not demonstrate systemic exposure. Formulation (13-2) were not well tolerated due to severe erythema noted.

### Dermal Observations

Skin reactions at the application sites were observed once daily prior to dosing. Dermal irritation was scored as indicated in Table 40.

TABLE 40
Scoring of skin reactions

| Erthyema and eschar | | | | Size of area of other | |
|---|---|---|---|---|---|
| formation | | Oedema formation | | reactions | |
| No erythema | 0 | No oedema | 0 | No skin | 0 |
| | | | | reactions | |
| Very slight | 1 | Very slight oedema | 1 | <10% of test | 1 |
| erythema (barely | | (barely perceptible) | | area | |
| perceptible) | | | | | |
| Well defined | 2 | Slight oedema (edges | 2 | 10-25% of test | 2 |
| erythema | | of area well defined by | | area | |
| | | definite raising) | | | |
| Moderate to severe | 3 | Moderate oedema | 3 | 26-50% of test | 3 |
| erythema | | (raised approximately | | area | |
| | | 1 mm) | | | |
| Severe erythema | 4 | Severe oedema (raised | 4 | 51-75% of test | 4 |
| (beet redness) to | | more than 1 mm, | | area | |
| eschar formation | | extending beyond area | | | |
| preventing grading | | of exposure) | | | |
| of erythema | | | | | |
| — | — | — | — | 76-100% of test | 5 |
| | | | | area | |

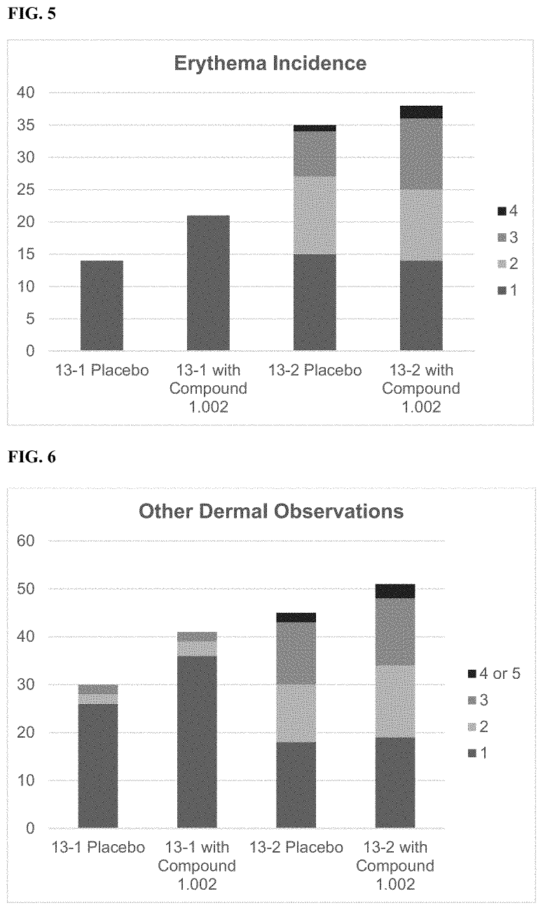

FIG. 5 shows incidence of erythema in minipigs treated with formulations (13-1) and (13-2). Similarly, FIG. 6 shows incidence of other dermal observations in minipigs treated with formulations (13-1) and (13-2). No erythema was noted in any animal at 1 or 2 weeks after dosing stopped, indicating that erythema recovered in all animals. Evidence of recovery for other dermal observations was also observed.

### Other Observations

Starting on arrival, animals were weighed approximately once weekly. The last pre-dose body weight was used for re-allocation. During the dosing period, animals were weighed on the first day of each study week (Days 1, 8, 15, etc.). Animals were weighed on the day of collection of biopsies. Food consumption was estimated daily for each animal from Day −7 by weighing unconsumed diet about 1 hour after feeding. On days with anaesthesia, animals had remains of diet available during the night and food consumption was estimated the following morning.

Blood samples were taken from all animals twice before the start of treatment. Animals were fasted (excepting water) overnight before taking blood samples. Blood samples were drawn from the jugular vein/bijugular trunk. Hematological analysis including blood chemistry analysis was performed. Toxicokinetic analyses were also performed.

Biopsies were performed at application sites using a biopsy puncher (6 millimeters in diameter) on the day following the last dosing (Day 29). In addition, one biopsy of untreated skin was collected. Biopsies were fixed in phosphate buffered neutral 4% formaldehyde.

Minimal to moderate epidermal hyperplasia with or without cellular infiltration was observed in some treated application areas. Only selected animals treated with formulation (13-1) showed no findings. No differences in severity were observed in left (L) and right (R) sites were observed. The lowest severity of epidermal hyperplasia in both left and right application areas was observed with formulation (13-1), while the highest severity was observed with formulation (13-2) for both sides.

### Findings

While each of the formulations assessed in the minipigs study was generally tolerated, it was determined that formulation (13-1) demonstrated the most acceptable tolerability profile and did not demonstrate systemic exposure. Formulation (13-1) met various criteria for a formulation for delivering compound 1.002 including dermal tolerability (e.g., acceptable severity and frequency of erythema, edmea, and other dermal observations as corroborated by microscopic findings, and must not cause dippity pig syndrome in pigs), stability, skin flux (e.g., at least 50 μg/cm² cumulative delivery between 24-48 hours), viscosity (between about 5,000-15,000 cP), dissolution, microbiological enumeration, and appearance. The composition of the formulation was refined as shown in Table 41 below. The vendors included in the table are suggestions; materials supplied from other vendors may also be suitable.

TABLE 41
Compositions of formulation (13-1) (i.e., Formulation (BA1)

| Ingredients | Vendor | Amounts | wt/wt % |
|---|---|---|---|
| DMSO | Gaylord | 5 | 5 |
| Oleyl alcohol | BASF new | 5 | 5 |
| Transcutol P | Gattefosse | 25 | 25 |
| Dipropylene glycol | Sigma | 5 | 5 |
| PEG400 | BASF | 10 | 10 |
| Propylene glycol | Spectrum | 50 | 50 |
| Total weight of the base | — | 100.0 | 100.0% |
| formulation | | | |
| HPC-H | Nisso | 1.7 | 1.7 |
| Compound 1.002 | — | 2.4 | 2.4 |

One or more additional components may be included in a formulation described above. For example, the addition of an acid (e.g., citric acid, acetic acid, or phosphoric acid) in an amount of less than about 1% by weight of the base formulation may contribute to stabilization of the formulation as outlined in the preceding examples without contributing to skin irritation.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A formulation, comprising:
a) a compound having formula (I): 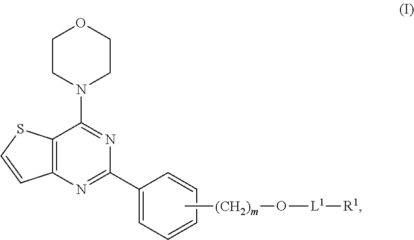
a hydrate, a solvate, a pharmaceutically acceptable salt, or a combination thereof;
wherein:
subscript m is an integer from 0 to 2;
L¹ is —C(O)—, —C(O)O—, —C(O)S—, or —C(O)NH—; and
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) a base formulation comprising:
b1) dimethyl sulfoxide (DMSO) in an amount of from about 0% to about 20% by weight of the base formulation;
b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
b3) one or more solvents comprising a C₂₋₆ alkylene glycol, a di-(C₂₋₆ alkylene) glycol, or a combination thereof; and
c) an acid in an amount of no more than about 1% by weight of the base formulation,
wherein
a hydrolysis of the compound having formula (I) to a corresponding compound having formula (IV): 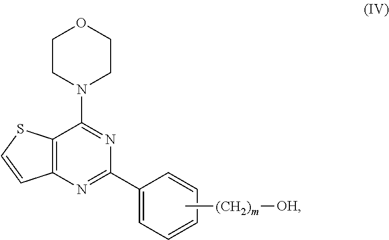
is less than about 10% over a period of about 10 days at a temperature of 80° C. (±2° C.) or over a period of about 6 months at a temperature of 40° C. (±2° C.) and a relative humidity of 75% (±5%), wherein subscript m is an integer from 0 to 2.

2. The formulation of claim 1, wherein the formulation is a topical formulation.

3. The formulation of claim 1, wherein the one or more excipients are oleyl alcohol in an amount of from about 3% to about 10% by weight of the base formulation.

4. The formulation of claim 3, wherein oleyl alcohol is present in an amount of about 5% by weight of the base formulation.

5. The formulation of claim 1, wherein DMSO, when present, is in an amount of from about 5% to about 20%, from about 5% to about 15%, from about 10% to about 15%, or from about 5% to about 10% by weight of the base formulation.

6. The formulation of claim 5, wherein DMSO is present in an amount of about 5% by weight of the base formulation.

7. (canceled)

8. The formulation of claim 1, wherein the acid is citric acid, acetic acid, or phosphoric acid.

9. The formulation of claim 8, wherein citric acid is present in an amount of from about 0.01% to about 0.1% by weight of the base formulation.

10. The formulation of claim 9, wherein citric acid is present in an amount of about 0.05% by weight of the base formulation.

11. The formulation of claim 8, wherein acetic acid is present in an amount of from about 0.01% to about 0.5% by weight of the base formulation.

12. The formulation of claim 11, wherein acetic acid is present in an amount of about 0.05% by weight of the base formulation.

13. The formulation of claim 8, wherein phosphoric acid is present in an amount of from about 0.001% to about 0.01% by weight of the base formulation.

14. The formulation of claim 13, wherein phosphoric acid is present in an amount of about 0.005% by weight of the base formulation.

15. (canceled)

16. The formulation of claim 1, wherein the one or more solvents comprise 2-(2-ethoxyethoxy)ethanol, propylene glycol, dipropylene glycol, and PEG400.

17. The formulation of claim 16, wherein 2-(2-ethoxyethoxy)ethanol is present in an amount of from about 20% to about 40%, from about 20% to about 35%, or from about 20% to about 30% by weight of the base formulation; propylene glycol is present in an amount of from about 30% to about 70%, from about 30% to about 60%, from about 40% to about 60%, or from about 45% to about 55% by weight of the base formulation; and/or dipropylene glycol is present in an amount of from about 1% to about 10% by weight of the base formulation.

18. The formulation of claim 17, wherein 2-(2-ethoxyethoxy)ethanol is present in an amount of about 25% by weight of the base formulation.

19. (canceled)

20. The formulation of claim 17, wherein propylene glycol is present in an amount of about 50% by weight of the base formulation.

21. (canceled)

22. The formulation of claim 17, wherein dipropylene glycol is present in an amount of about 5% by weight of the base formulation.

23. The formulation of claim 16, wherein, when DMSO is present, PEG400 is present in an amount of from about 5% to about 15% by weight of the base formulation.

24. The formulation of claim 23, wherein PEG400 is present in an amount of about 10% by weight of the base formulation.

25. The formulation of claim 1, further comprising a gelling agent.

26. The formulation of claim 25, wherein the gelling agent is hydroxypropyl cellulose.

27. The formulation of claim 1, comprising:
a) the compound of formula (I);
b) a base formulation comprising:
b1) DMSO in an amount of about 5% by weight of the base formulation;
b2) oleyl alcohol in an amount of about 5% by weight of the base formulation;
b3) 2-(2-ethoxyethoxy)ethanol, propylene glycol, dipropylene glycol, and PEG400, which are present in an amount of about 25%, about 50%, about 5%, and about 10%, respectively, by weight of the base formulation;
c) citric acid in an amount of about 0.05% by weight of the base formulation; and
d) hydroxypropyl cellulose in an amount of from about 0.5% to about 2% by weight of the base formulation, wherein the hydroxypropyl cellulose has an average molecular weight of from about 700,000 Da to about 1,150,000 Da.

28. The formulation of claim 1, comprising:
a) the compound of formula (I);
b) a base formulation comprising:
b1) DMSO in an amount of about 5% by weight of the base formulation;
b2) oleyl alcohol in an amount of about 3% by weight of the base formulation;
b3) 2-(2-ethoxyethoxy)ethanol, propylene glycol, dipropylene glycol, and PEG400, which are present in an amount of about 25%, about 52%, about 5%, and about 10%, respectively, by weight of the base formulation;
c) citric acid in an amount of about 0.05% by weight of the base formulation; and
d) hydroxypropyl cellulose in an amount of about 0.5% to about 2% by weight of the base formulation, wherein the hydroxypropyl cellulose has an average molecular weight of from about 700,000 Da to about 1,150,000 Da.

29. The formulation of claim 1, wherein compound of formula (I) is present in a degree to saturation of from about 75% to about 100% or from about 90% to about 100%.

30. The formulation of claim 1, wherein the formulation has an apparent pH value of from about 4 to about 5.

31. The formulation of claim 1, wherein a skin flux of the compound of formula (I) has an increase of greater than about 2 fold as compared to a skin flux of the corresponding compound of formula (IV) in the same formulation, provided that the compound of formula (I) and the compound of formula (IV) have the same degree to saturation.

32. The formulation of claim 1, wherein the compound of formula (I) is represented by formula (IIa-1a):

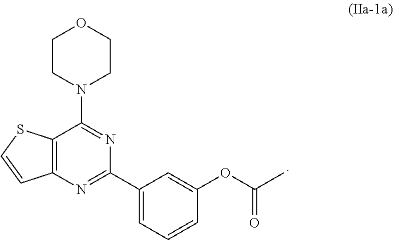 [SMILES: CC(=O)Oc1cccc(-c2nc(N3CCOCC3)c3sccc3n2)c1]

33. The formulation of claim 32, wherein a cumulative skin flux of the compound of formula (IIa-1a) is at least about 3 μg/cm²/hour at about 24 hours, as measured by a Franz diffusion cell using a human cadaver skin.

34. The formulation of claim 1, wherein the formulation is in a form of a foam, a lotion, a pray, an aerosol, an ointment, a cream, a gel, a paste, a patch, or an in-situ patch.

35. A process for preparing a formulation according to claim 1, comprising:
1) forming a first mixture comprising:
a) a compound having formula (I): 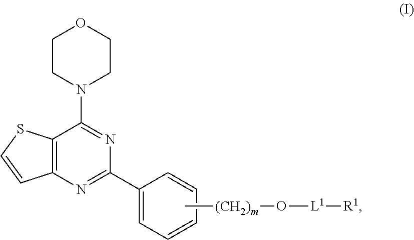
a hydrate, a solvate, a pharmaceutically acceptable salt, or a combination thereof;
wherein:
subscript m is an integer from 0 to 2;
L¹ is —C(O)—, —C(O)O—, —C(O)S—, or —C(O)NH—; and
R¹ is C₁₋₆ alkyl, C₁₋₆ hydroxyalkyl, C₁₋₆ haloalkyl, C₂₋₆ alkenyl, C₆₋₁₀ aryl, C₆₋₁₀ aryl-C₁₋₆ alkyl, or C₆₋₁₀ aryl-C₂₋₆ alkenyl;
b) a base formulation comprising:
b1) dimethyl sulfoxide (DMSO) in an amount of from about 0% to about 20% by weight of the base formulation;
b2) one or more excipients selected from the group consisting of an unsaturated fatty alcohol, an unsaturated fatty acid, an unsaturated fatty ester, an unsaturated fatty ether, and a combination thereof, wherein the one or more excipients are in an amount of no more than about 10% by weight of the base formulation; and
b3) one or more solvents comprising a C₂₋₆ alkylene glycol, a di-(C₂₋₆ alkylene) glycol, or a combination thereof; and
c) an acid in an amount of no more than about 1% by weight of the base formulation, and
2) mixing the first mixture form a uniform mixture.

36-37. (canceled)

38. A formulation, prepared by a process according to claim 35.

39. A method of treating a vascular malformation in a subject in need thereof, comprising topically administering to the subject an effective amount of the formulation of claim 1.

40-43. (canceled)
